(19) United States
(12) Patent Application Publication
(10) Pub. No.: US 2008/0019968 A1
(43) Pub. Date: Jan. 24, 2008

(54) DETECTION, PREVENTION AND TREATMENT OF BREAST CANCER

(75) Inventors: Ola Blixt, La Jolla, CA (US); Massimo Cristofanilli, Pearland, TX (US); Margaret E. Huflejt, La Jolla, CA (US)

Correspondence Address: SCHWEGMAN, LUNDBERG & WOESSNER, P.A., P.O. BOX 2938, MINNEAPOLIS, MN, 55402, US

(73) Assignees: The scripps Reasearch Institute, La Jolla, CA (US); Board Of Regents The University of Texas System, austin, TX (US)

(21) Appl. No.: 11/750,923

(22) Filed: May 18, 2007

Related U.S. Application Data
(63) Continuation of application No. PCT/US2005/042373, filed on Nov. 21, 2005.
(60) Provisional application No. 60/629,666, filed on Nov. 19, 2004.

(51) Int. Cl. A61K 39/395; A61K 39/00; A61P 43/00; C07K 14/00; C40B 30/04; C40B 40/10

(52) U.S. Cl. 424/138.1; 424/184.1; 506/18; 506/9; 530/395

(57) ABSTRACT

The invention provides compositions and arrays of glycans for detecting, treating and monitoring breast cancer in a human or other mammal.

### RELATED APPLICATIONS

This application is a continuation under 35 U.S.C. 111(a) of International Application No. PCT/US2005/042373 filed 21 Nov. 2005 and published as WO 2006/068758 A3 on 29 Jun. 2006, which claimed the benefit of the filing date of U.S. Provisional Ser. No. 60/629,666 filed 19 Nov. 2004, the contents of which applications and publication are incorporated herein by reference.

This application is also related to U.S. Provisional Ser. No. 60/550,667, filed Mar. 5, 2004, and U.S. Provisional Ser. No. 60/558,598, filed Mar. 31, 2004, and U.S. patent application Ser. No. 11/516,014 filed Sep. 5, 2006, the contents of which are also incorporated herein by reference.

### GOVERNMENT FUNDING

The invention described herein was made with United States Government support under Grant Number U54GM62116 awarded by the National Institutes of Health. The United States Government has certain rights in this invention.

### FIELD OF THE INVENTION

The invention relates to glycan arrays and methods for detecting breast cancer using those glycan arrays. In another embodiment, the invention provides glycan compositions useful for treating and prevention breast cancer. These glycan compositions can be used to generate an immune response against breast cancer cell epitopes. Alternatively, the glycan compositions can be used to prepare antibody preparations useful for passive immunization against breast cancer.

### BACKGROUND OF THE INVENTION

Glycans are typically the first and potentially the most important interface between cells and their environment. As vital constituents of all living systems, glycans are involved in recognition, adherence, motility and signaling processes. There are at least three reasons why glycans should be studied: (1) all cells in living organisms, and viruses, are coated with diverse types of glycans; (2) glycosylation is a form of post- or co-translational modification occurring in all living organisms; and (3) altered glycosylation is an indication of an early and possibly critical point in development of human pathologies. Jun Hirabayashi, Oligosaccharide microarrays for glycomics; 2003, Trends in Biotechnology. 21 (4): 141-143; Sen-Itiroh Hakomori, Tumor-associated carbohydrate antigens defining tumor malignancy: Basis for development of and cancer vaccines; in The Molecular Immunology of Complex Carbohydrates-2 (Albert M Wu, ed., Kluwer Academic/Plenum, 2001). These cell-identifying glycosylated molecules include glycoproteins and glycolipids and are specifically recognized by various glycan-recognition proteins, called ‘lectins.’ However, the enormous complexity of these interactions, and the lack of well-defined glycan libraries and analytical methods have been major obstacles in the development of glycomics.

The development of nucleotide and protein microarrays has revolutionized genomic, gene expression and proteomic research. While the pace of innovation of these arrays has been explosive, the development of glycan microarrays has been relatively slow. One reason for this is that it has been difficult to reliably immobilize populations of chemically and structurally diverse glycans. Moreover, glycans are not readily amenable to analysis by many of the currently available molecular techniques (such as rapid sequencing and in vitro synthesis) that are routinely applied to nucleic acids and proteins. However, the use of glycan arrays could expedite screening procedures, making detection of cancer-related glycan epitopes simple and inexpensive.

For example, breast cancer is the most common cancer in women, with well over 200,000 new cases being diagnosed each year. In the United States, one out of every eight women will eventually be diagnosed with breast cancer. Although many treatments have been developed over the years, effective treatment still relies largely on early detection of the disease. Even greater numbers of women, however, have symptoms associated with breast diseases, both benign and malignant, and must undergo further diagnosis and evaluation in order to determine whether breast cancer exists. To that end, a variety of diagnostic techniques have been developed, the most common of which are surgical techniques including core biopsy and excisional biopsy. Recently, fine needle aspiration (FNA) cytology has been developed which is less invasive than the surgical techniques, but which is not always a substitute for surgical biopsy. However, these techniques are still uncomfortable and invasive.

Thus, new non-invasive tools and procedures are needed to expedite early detection of breast cancer. Also, improved methods for treating and preventing breast cancer are needed.

### SUMMARY OF THE INVENTION

The invention provides glycan libraries, glycan arrays (or microarrays) and methods for using such arrays to detect and diagnose breast cancer. According to the invention, patients with breast cancer have circulating antibodies that react with a subset of glycan epitopes. Unlike such breast cancer patients, people without breast cancer have substantially no circulating antibodies that react with those glycan epitopes.

Therefore, one aspect of the invention is a breast cancer epitope selected from the group consisting of ceruloplasmin, Neu5Gc(2-6)GalNAc, GM1, Sulfo-T, Globo-H, sialylated Tn (Neu5Ac-alpha6-GalNAc-alpha) and LNT-2 glycans. In another embodiment, the invention is directed to a breast cancer epitope selected from the group consisting of Tri-LacNAc (glycan 9), LacNAc-LeX-LeX (glycan 73), LacNAc-LacNAc (glycan 76), H-type-2-LacNAc (glycan 106), H-type2-LacNAc-LacNAc (glycan 107), GlcNAcβ3LacNAc (glycan 124), SLeXLacNAc (glycan 174), 3′SialylDiLacNAc (glycan 179), 3′Sialyl-tri-LacNAc (glycan 180), 6Sia-LacNAc-LeX-LeX (glycan 188), 6SiaLacNAc-LacNAc (glycan 189). The glycan numbers correspond to the glycans listed in Table 1. The structures of these glycans are shown in FIG. 11, where the linker (e.g. SP1) may or may not be present in the breast cancer epitope.

Another aspect of the invention is a method of detecting breast cancer, or a propensity to develop breast cancer, in a patient. The method involves contacting a test sample obtained from the patient with a glycan library or glycan array of the invention, and observing whether antibodies in the test sample bind to glycans in the library or the array. According to the invention, the type of glycan bound by such antibodies is indicative of the presence of breast cancer, or the propensity to develop breast cancer, or the invasiveness or malignancy of the breast cancer in the patient. The binding pattern of test samples can be compared to the binding of control samples from individuals without cancer or malignancy. Alternatively, the control sample can be a sample obtained from the patient prior to development of the cancer or, for purposes of monitoring the cancer, at a time when the cancer was more or less aggressive. The test and control samples can, for example, be blood samples, serum samples, plasma samples, urine samples, breast milk samples, breast secretion samples, nipple aspirates, ascites fluids, plural ascites fluids, saliva samples, cerebrospinal fluids, vaginal secretions, ovarian fluids or a tissue sample. One convenient sample type for use in the invention is serum.

In some embodiments, the methods of the invention are useful for identifying the particular glycan profile bound by antibodies present in patient samples. The types of glycans bound by these antibodies is indicative of the type, extent and/or prognosis of the disease. Thus, low-risk types of breast cancer as well as more aggressive types of breast cancer can be detected using the present methods. According to the invention, patients with breast cancer have circulating antibodies that react with glycans such as ceruloplasmin glycans, Neu5Acα2-6GalNAcα, certain T-antigens carrying various substituents and other modifications, LNT-2 (a known ligand for tumor-promoting Galectin-4; see Huflejt & Leffler (2004). Glycoconjugate J, 20: 247-255), Globo-H—, and GM1-antigens. GM1 is a glycan that includes the following carbohydrate structure: Gal-beta3-GalNAc-beta4-[Neu5Ac-alpha3]-Gal-beta-4-Glc-beta. Sulfo-T is a T-antigen with sulfate residues, for example, Sulfo-T can include a carbohydrate of the following structure: Galβ3GalNAc. Globo-His a glycan that includes the following carbohydrate structure: Fucose-alpha2-Gal-beta3-GalNAc-beta3-Gal-alpha-4-Gal-beta-4-Glc. LNT-2 is a glycan that includes the following carbohydrate structure: GlcNAc-beta3-Gal-beta-4-Glc-beta. Sialylated Tn is a glycan with the following structure: Neu5Ac-alpha6-GalNAc-alpha. Circulating antibodies from breast cancer patients can also react with the following glycans: Tri-LacNAc (glycan 9 of Table 1), LacNAc-LeX-LeX (glycan 73), LacNAc-LacNAc (glycan 76), H-type-2-LacNAc (glycan 106), H-type2-LacNAc-LacNAc (glycan 107), GlcNAcβ3LacNAc (glycan 124), SLeXLacNAc (glycan 174), 3′SialylDiLacNAc (glycan 179), 3′Sialyl-tri-LacNAc (glycan 180), 6Sia-LacNAc-LeX-LeX (glycan 188), 6SiaLacNAc-LacNAc (glycan 189). The glycan numbers correspond to the glycans listed in Table 1. The structures of these glycans are shown in FIG. 11, where the linker (e.g. SP1) may or may not be present on the glycan.

The glycans used on the arrays for detecting breast cancer include glycans that react with antibodies associated neoplasia in sera of mammals with benign or pre-malignant tumors. Such glycans have two or more sugar units. The glycans of the invention include straight chain and branched oligosaccharides as well as naturally occurring and synthetic glycans. Any type of sugar unit can be present in the glycans of the invention, including allose, altrose, arabinose, glucose, galactose, gulose, fucose, fructose, idose, lyxose, mannose, ribose, talose, xylose, neuraminic acid or other sugar units. Such sugar units can have a variety of substituents. For example, substituents that can be present instead of, or in addition to, the substituents typically present on the sugar units include amino, carboxy, thiol, azide, N-acetyl, N-acetylneuraminic acid, oxy (═O), sialic acid, sulfate (—SO₄⁻), phosphate (—PO₄⁻), lower alkoxy, lower alkanoyloxy, lower acyl, and/or lower alkanoylaminoalkyl. Fatty acids, lipids, amino acids, peptides and proteins can also be attached to the glycans of the invention.

In another embodiment, the invention provides an array or a microarray for detecting breast cancer that includes a solid support and a multitude of defined glycan probe locations on the solid support, each glycan probe location defining a region of the solid support that has multiple copies of one type of glycan molecule attached thereto and wherein the glycans are attached to the microarray by a cleavable linker. These microarrays can have, for example, between about 2 to about 100,000 different glycan probe locations, or between about 2 to about 10,000 different glycan probe locations. Glycans selected for use in the arrays or microarrays include those that react with antibodies associated with breast neoplasia that are present in sera and/or ascites fluid of humans and other mammals with benign, pre-malignant or malignant tumors.

In another embodiment, the invention provides a composition that includes a carrier and at least one glycan that reacts with antibodies associated with neoplasia in sera or bodily fluids of humans or other mammals with benign, pre-malignant or malignant breast tumors. These compositions can be formulated for immunization of a human or other mammal. Alternatively, these compositions can be formulated in a food supplement. The compositions of the invention are useful for treating and preventing breast cancer.

In another embodiment, the invention provides a method of identifying whether a patient or a mammal has breast cancer that includes contacting an array or library of the invention with a test sample and observing whether antibodies in the test sample bind to glycans that react with antibodies associated with neoplasia in sera and/or ascites fluid of humans and other mammals with benign, pre-malignant or malignant tumors.

In another embodiment, the invention provides a method of treating or preventing breast cancer in a human or other mammal that comprises administering to the mammal a composition comprising an effective amount of at least one glycan molecule that binds antibodies associated with neoplasia in sera and/or ascites fluid of humans and other mammals with benign, pre-malignant or malignant tumors.

In another embodiment, the invention provides an isolated antibody that can bind to a glycan that can react with antibodies associated with neoplasia that are present in sera or bodily fluids of humans and other mammals with benign, pre-malignant or malignant tumors.

### DESCRIPTION OF THE FIGURES

FIG. 1 illustrates covalent printing of a diverse glycan library onto an amino-reactive glass surface and image analysis using standard microarray technology.

FIG. 2 provides representative glycan structures on an array. Glycan structures detected by glycan binding proteins are shown in the symbol nomenclature adopted by the Consortium for Functional Glycomics (http://www.functionalglycomics.org). Some of the symbols employed are described below:

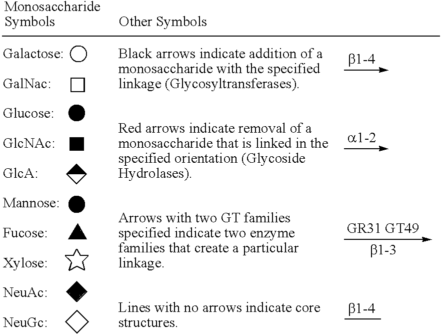

A more complete list of glycans used in the arrays of the invention can be found in FIG. 7 and further description of the types of saccharides, saccharide derivatives and saccharide linkages employed can be found in the tables and text provided herein.

FIG. 3 provides data illustrating printing optimization and the specificity of selected plant lectins. FIG. 3A provides a graph relating the glycan concentration and length of printing time to the relative fluorescence of the signal detected from binding Con A-FITC. Optimized glycan concentrations and printing times were determined by printing selected mannose glycan structures and then detecting Con A binding thereto. A representative mannose glycan (136, see FIG. 7) was printed at various concentrations (4 μM-500 μM) in replicates of eight at six different time points. FIG. 3B illustrates the binding specificities of Con A-FITC and ECA-FITC on the complete array of glycans whose structures are provided in FIG. 7.

FIG. 4 illustrates the specificity of mammalian glycan binding proteins on a glycan array of the invention. C-Type lectin (DC-SIGN): DC-SIGN-Fc chimera (30 μg/mL) detected by secondary goat anti-human-IgG-Alexa-488 antibody (10 μg/mL) bound selectively to α1-2- and/or α1-3/4-fucosylated glycans as well as to Manα1-2-glycans. Siglec (CD22): CD22-Fc chimera (10 μg/mL) pre-complexed with secondary goat anti-human-IgG-Alexa-488 (5 μg/mL) and tertiary rabbit anti-goat-IgG-FITC (2.5 μg/mL) antibodies bound exclusively to Neu5Acα2-6Gal-glycans. Galectin (Galectin-4): Human Galectin-4-Alexa488 (10 μg/mL) evaluated with glycans printed at 100 μM (100 μM) and at 10 μM (10 μM) bound preferentially to blood group glycans.

FIG. 5 illustrates the specificity of various anti-carbohydrate antibodies on the glycan arrays of the invention. Anti-CD15: Mouse anti-CD15-FITC monoclonal antibody (BD Biosciences Clone HI98, 100 tests) bound exclusively to Lewis^{X} glycans. Human anti-HIV 2G12: 2G12 monoclonal antibody (30 μg/mL) pre-complexed with goat anti-human-IgG-FITC (15 μg/mL) bound to specific Manα1-2-glycans including the Man8 and Man9 N-glycans. Human Serum: Human serum of ten healthy individuals (1:25 dilution) were individually bound to glycan arrays and detected by subsequent overlay with monoclonal mouse anti-human-IgG-IgM-IgA-Biotin antibody (10 μg/mL) and Streptavidin-FITC (10 μg/mL) respectively. Results represent the mean and standard deviation for binding in all ten experiments. Anti-carbohydrate antibodies detecting various blood group antigens as well as mannans and bacterial fragments were found.

FIG. 6 illustrates the specificity of various bacterial and viral glycan binding proteins for certain glycans in the arrays of the invention. Cyanovirin-N: Cyanovirin-N (30 μg/mL) detected with secondary polyclonal rabbit anti-CVN (10 μg/mL) and tertiary anti-rabbit-IgG-FITC (10 μg/mL) bound various a 1-2 mannosides. Influenza H3 hemagglutinin: Pure recombinant hemagglutinin (150 μg/mL) derived from Duck/Ukraine/1/63 (H3/N7), pre-complexed with mouse anti-HisTag-IgG-Alexa-488 (75 μg/mL) and anti-mouse-IgG-Alexa-488 (35 μg/mL), bound exclusively to Neu5Acα2-3Gal-terminating glycans. Influenza virus: Intact influenza virus A/Puerto Rico/8/34 (H1N1) was applied at 100 μg/ml in the presence of 10 μM of the neuraminidase inhibitor oseltamivir carboxylate. The virus bound a wide spectrum of sialosides with both NeuAcα2-3Gal and NeuAcα2-6Gal sequences.

the covalent attachment of an amino-functionalized glycan library to an N-hydroxysuccinimide (NHS) derivatized surface of a glass microarray.

FIG. 7 provides a schematic diagram of glycans used in some of the glycan arrays of the invention.

FIG. 8 provides a bar graph illustrating which glycans react with anti-carbohydrate antibodies found in sera of metastatic breast cancer patients. Each bar represents the relative fluorescence intensity of a given anti-glycan antibody in an individual patient. Red bars represent the intensities observed for reaction of metastatic breast cancer patient serum with background (# 1, a negative control), ceruloplasmin (#2), Neu5Gc(2-6)GalNAc (#3), Neu5Ac(2-6)GalNAc (#4), GMI (#5), Sulfo-T (#6), Globo-H (#7), LNT-2 (#8) and Rhamnose (#10, a positive control). Orange bars, which are the tenth bar in each cluster of bars, represent the average values for metastatic cancer patients 1-9. Yellow bars, which are the eleventh bars in each cluster or bars, represent the average values for non-metastatic breast cancer patients. Blue bars, which are the twelfth through twenty-first bars, represent the average values of “healthy” individuals. Dark blue bars, which are twenty-second bars in each cluster of bars, represent the average values for healthy patients 12-21.

FIG. 9 provides a bar graph illustrating the additive relative fluorescence levels of selected breast cancer-associated anti-glycan antibodies in cancer (N=9) and non-cancer patients (N=10). The types of glycans that react with these antibodies are shown with the number of patients whose sera react with the indicated glycan. The inset provides a combined relative fluorescence levels for a group of known cancer-associated T-antigens carrying various modifications in metastatic breast cancer patients (1) and in “healthy” individuals (2).

FIG. 10 provides a bar graph illustrating the combined levels of tumor associated anti-glycan antibodies (from FIG. 9) in individual breast cancer patients. Red bars represent the combined signal observed for each individual metastatic cancer patient. Blue bars represent the combined signal observed for each individual non-cancer patient.

FIG. 11 provides structures of some of the glycans useful for detecting and treating breast cancer.

### DETAILED DESCRIPTION OF THE INVENTION

The invention provides libraries and arrays of glycans that can be used for detecting breast cancer. According to the invention, breast cancer patients, particularly breast cancer patients with metastatic breast cancer, have circulating antibodies that react with cancer-related epitopes and many of those epitopes are glycans. The detection of such antibodies in a patient is indicative of breast cancer, or the propensity to develop breast cancer. Thus, women exhibiting mammographic abnormalities can be quickly tested using the libraries, arrays and methods of the invention to ascertain whether they have no risk or a low risk or a high risk of developing invasive breast cancer. In some embodiments, the presence of such antibodies is indicative of the presence of established invasive breast cancer and can provide information on the prognosis of such an established disease. Patients with familial history of breast cancer, and hence a heightened risk of developing the disease, can be tested regularly to monitor their propensity for disease.

Another aspect of the invention is a composition of glycans that can be used for treating or preventing breast cancer. The compositions include glycans that react with circulating antibodies found in breast cancer patients. The compositions can be used to elicit protective immune response in patients with a high risk of developing breast malignancies. The compositions can also be used to enhance the immune response of patients that have breast cancer. The compositions can also be used to prepare isolated antibody preparations useful for passive immunization of patients who have developed or may develop breast cancer.

The following abbreviations may be used herein: α₁-AGP means alpha-acid glycoprotein; AF488 means AlexaFluour-488; CFG means Consortium for Functional Glycomics; Con A means Concanavalin A; CVN means cyanovirin-N; DC-SIGN means dendritic cell-specific ICAM-grabbing nonintegrin; ECA means Erythrina cristagalli; ELISA means enzyme-linked immunosorbent assay; FITC means Fluorescinisothiocyanate; GBP means Glycan Binding Protein; HIV means human immunodeficiency virus; HA means influenza hemagglutinin; NHS means N-hydroxysuccinimide; PBS means phosphate buffered saline; SDS means sodium dodecyl sulfate; SEM means standard error of mean; and Siglec means sialic acid immunoglobulin superfamily lectins.

A “defined glycan probe location” as used herein is a predefined region of a solid support to which a density of glycan molecules, all having similar glycan structures, is attached. The terms “glycan region,” or “selected region”, or simply “region” are used interchangeably herein for the term defined glycan probe location. The defined glycan probe location may have any convenient shape, for example, circular, rectangular, elliptical, wedge-shaped, and the like. In some embodiments, a defined glycan probe location and, therefore, the area upon which each distinct glycan type or a distinct group of structurally related glycans is attached is smaller than about 1 cm², or less than 1 mm², or less than 0.5 mm². In some embodiments the glycan probe locations have an area less than about 10,000 im² or less than 100 im². The glycan molecules attached within each defined glycan probe location are substantially identical. Additionally, multiple copies of each glycan type are present within each defined glycan probe location. The number of copies of each glycan types within each defined glycan probe location can be in the thousands to the millions.

As used herein, the arrays of the invention have defined glycan probe locations, each with “one type of glycan molecule.” The “one type of glycan molecule” employed can be a group of substantially structurally identical glycan molecules or a group of structurally similar glycan molecules. There is no need for every glycan molecule within a defined glycan probe location to have an identical structure. In some embodiments, the glycans within a single defined glycan probe location are structural isomers, have variable numbers of sugar units or are branched in somewhat different ways. However, in general, the glycans within a defined glycan probe location have substantially the same type of sugar units and/or approximately the same proportion of each type of sugar unit. The types of substituents on the sugar units of the glycans within a defined glycan probe location are also substantially the same.

As used herein a “patient” is a mammal. Such mammals include domesticated animals, animals used in experiments, zoo animals and the like. For example, the patient can be a dog, cat, monkey, horse, rat, mouse, rabbit, goat, ape or human mammal. In many embodiments, the patient is a human.

Some of the structural elements of the glycans described herein are referenced in abbreviated form. Many of the abbreviations used are provided in the following table. Moreover the glycans of the invention can have any of the sugar units, monosaccharides or core structures provided in this table.

| Trivial Name | Monosaccharide/Core | Code |
|---|---|---|
| D-Glcp | D-Glucopyranose | G |
| D-Galp | D-Galactopyranose | A |
| D-GlcpNAc | N-Acetylglucopyranose | GN |
| D-GlcpN | D-Glucosamine | GQ |
| D-GalpNAc | N-Acetylgalactopyranose | AN |
| D-GalpN | D-Galacosamine | AQ |
| D-Manp | D-Mannopyranose | M |
| D-ManpNAc | D-NJ-Acetylmannopyranose | MN |
| D-Neup5Ac | N-Acetylneuraminic acid | NN |
| D-Neu5G | D-N-Glycolylneuraminic acid | NJ |
| D-Neup | Neuraminic acid | N |
| KDN* | 2-Keto-3-deoxynananic acid | K |
| Kdo | 3-deoxy-D-manno-2 | W |
| | octulopyranosylono | |
| D-GalpA | D-Galactoronic acid | L |
| D-Idop | D-Iodoronic acid | I |
| L-Rhap | L-Rhamnopyranose | H |
| L-Fucp | L-Fucopyranose | F |
| D-Xylp | D-Xylopyranose | X |
| D-Ribp | D-Ribopyranose | B |
| L-Araf | L-Arabinofuranose | R |
| D-GlcpA | D-Glucoronic acid | U |
| D-Allp | D-Allopyranose | O |
| D-Apip | D-Apiopyranose | P |
| D-Tagp | D-Tagopyranose | T |
| D-Abep | D-Abequopyranose | Q |
| D-Xulp | D-Xylulopyranose | D |
| D-Fruf | D-Fructofuranose | E |

*Another description of KDN is: 3-deoxy-D-glycero-K-galacto-nonulosonic acid

The sugar units or other saccharide structures present in the glycans of the invention can be chemically modified in a variety of ways. A listing of some of the types of modifications and substituents that the sugar units in the glycans of the invention can possess, along with the abbreviations for these modifications/substituents are listed below.

| Modification type | Symbol | Modification type | Symbol |
|---|---|---|---|
| Acid | A | Acid | A |
| N-Methylcarbamoyl | ECO | deacetylated N-Acetyl | Q |
| | | (amine) | |
| pentyl | EE | Deoxy | Y |
| octyl | EH | Ethyl | ET |
| ethyl | ET | Hydroxyl | OH |
| inositol | IN | Inositol | IN |
| N-Glycolyl | J | Methyl | ME |
| methyl | ME | N-Acetyl | N |
| N-Acetyl | N | N-Glycolyl | J |
| hydroxyl | OH | N-Methylcarbamoyl | ECO |
| phosphate | P | N-Sulfate | QS |
| phosphocholine | PC | O-Acetyl | T |
| Phosphoethanolamine (2- | PE | Octyl | EH |
| aminoethylphosphate) | | Pentyl | EE |
| Pyrovat acetal | PYR* | Phosphate | P |
| deacetylated N-Acetyl | Q | Phosphocholine | PC |
| (amine) | | | |
| N-Sulfate | QS | Phosphoethanolamine (2- | PE |
| sulfate | S | aminoethylphosphate) | |
| O-Acetyl | T | Pyrovat acetal | PYR* |
| deoxy | Y | Sulfate | S |

when written on position 3, it means 3, 4, when to 4 it means 4, 6.

Glycans

The invention provides libraries of glycans that are useful for detecting and preventing breast cancer. These glycan libraries include numerous different types of carbohydrates and oligosaccharides. In general, the major structural attributes and composition of the separate glycans within the libraries have been identified. In some embodiments, the libraries consist of separate, substantially pure pools of glycans, carbohydrates and/or oligosaccharides. Further description of the types of glycans useful in the practice of the invention is provided in U.S. Provisional Ser. No. 60/550,667, filed Mar. 5, 2004, and U.S. Provisional Ser. No. 60/558,598, filed Mar. 31, 2004, the contents of which are incorporated herein by reference.

The glycans of the invention include straight chain and branched oligosaccharides as well as naturally occurring and synthetic glycans. For example, the glycan can be a glycoaminoacid, a glycopeptide, a glycolipid, a glycoaminoglycan (GAG), a glycoprotein, a whole cell, a cellular component, a glycoconjugate, a glycomimetic, a glycophospholipid anchor (GPI), glycosyl phosphatidylinositol (GPI)-linked glycoconjugates, bacterial lipopolysaccharides and endotoxins. The glycans can also include N-glycans, O-glycans, glycolipids and glycoproteins.

The glycans of the invention include 2 or more sugar units. Any type of sugar unit can be present in the glycans of the invention, including, for example, allose, altrose, arabinose, glucose, galactose, gulose, fucose, fructose, idose, lyxose, mannose, ribose, talose, xylose, or other sugar units. The tables provided herein list other examples of sugar units that can be used in the glycans of the invention. Such sugar units can have a variety of modifications and substituents. Some examples of the types of modifications and substituents contemplated are provided in the tables herein. For example, sugar units can have a variety of substituents in place of the hydroxy (—OH), carboxylate (—COO⁻), and methylenehydroxy (—CH₂—OH) substituents. Thus, lower alkyl moieties can replace any of the hydrogen atoms from the hydroxy (—OH), carboxylic acid (—COOH) and methylenehydroxy (—CH₂—OH) substituents of the sugar units in the glycans of the invention. For example, amino acetyl (—NH—CO—CH₃) can replace any of the hydrogen atoms from the hydroxy (—OH), carboxylic acid (—COOH) and methylenehydroxy (—CH₂—OH) substituents of the sugar units in the glycans of the invention. N-acetylneuraminic acid can replace any of the hydrogen atoms from the hydroxy (—OH), carboxylic acid (—COOH) and methylenehydroxy (—CH₂—OH) substituents of the sugar units in the glycans of the invention. Sialic acid can replace any of the hydrogen atoms from the hydroxy (—OH), carboxylic acid (—COOH) and methylenehydroxy (—CH₂—OH) substituents of the sugar units in the glycans of the invention. Amino or lower alkylamino groups can replace any of the OH groups on the hydroxy (—OH), carboxylic acid (—COOH) and methylenehydroxy (—CH₂—OH) substituents of the sugar units in the glycans of the invention. Sulfate (—SO₄⁻) or phosphate (—PO₄⁻) can replace any of the OH groups on the hydroxy (—OH), carboxylic acid (—COOH) and methylenehydroxy (—CH₂—OH) substituents of the sugar units in the glycans of the invention. Hence, substituents that can be present instead of, or in addition to, the substituents typically present on the sugar units include N-acetyl, N-acetylneuraminic acid, oxy (═O), sialic acid, sulfate (—SO₄⁻), phosphate (—PO₄⁻), lower alkoxy, lower alkanoyloxy, lower acyl, and/or lower alkanoylaminoalkyl.

The following definitions are used, unless otherwise described: Alkyl, alkoxy, alkenyl, alkynyl, etc. denote both straight and branched groups; but reference to an individual radical such as “propyl” embraces only the straight chain radical, when a branched chain isomer such as “isopropyl” has been specifically referred to. Halo is fluoro, chloro, bromo, or iodo.

Specifically, lower alkyl refers to (C₁-C₆)alkyl, which can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy.

It will be appreciated by those skilled in the art that the glycans of the invention having one or more chiral centers may exist in and be isolated in optically active and racemic forms. Some compounds may exhibit polymorphism. It is to be understood that the present invention encompasses any racemic, optically-active, polymorphic, or stereoisomeric form, or mixtures thereof, of a glycan of the invention, it being well known in the art how to prepare optically active forms (for example, by resolution of the racemic form by recrystallization techniques, by synthesis from optically-active starting materials, by chiral synthesis, or by chromatographic separation using a chiral stationary phase).

Specific and preferred values listed below for substituents and ranges, are for illustration only; they do not exclude other defined values or other values within defined ranges or for the substituents.

The libraries of the invention are particularly useful because diverse glycan structures are difficult to make and substantially pure solutions of a single glycan type are hard to generate. For example, because the sugar units typically present in glycans have several hydroxyl (—OH) groups and each of those hydroxyl groups is substantially of equal chemical reactivity, manipulation of a single selected hydroxyl group is difficult. Blocking one hydroxyl group and leaving one free is not trivial and requires a carefully designed series of reactions to obtain the desired regioselectivity and stereoselectivity. Moreover, the number of manipulations required increases with the size of the oligosaccharide. Hence, while synthesis of a disaccharide may require 5 to 12 steps, as many as 40 chemical steps can be involved in synthesis of a typical tetrasaccharide. In the past, chemical synthesis of oligosaccharides was therefore fraught with purification problems, low yields and high costs. However the invention has solved these problems by providing libraries and arrays of numerous structurally distinct glycans.

The glycans of the invention have been obtained by a variety of procedures. For example, some of the chemical approaches developed to prepare N-acetyllactosamines by glycosylation between derivatives of galactose and N-acetylglucosamine are described in Aly, M. R. E.; Ibrahim, E.-S. I.; El-Ashry, E.-S. H. E. and Schmidt, R. R., Carbohydr. Res. 1999, 316, 121-132; Ding, Y.; Fukuda, M. and Hindsgaul, O., Bioorg. Med. Chem. Lett. 1998, 8, 1903-1908; Kretzschmar, G. and Stahl, W., Tetrahedr. 1998, 54, 6341-6358. These procedures can be used to make the glycans of the present libraries, but because there are multiple tedious protection/deprotection steps involved in such chemical syntheses, the amounts of products obtained in these methods can be low, for example, in milligram quantities.

One way to avoid protection-deprotection steps typically required during glycan synthesis is to mimic nature's way of synthesizing oligosaccharides by using regiospecific and stereospecific enzymes, called glycosyltransferases, for coupling reactions between the monosaccharides. These enzymes catalyze the transfer of a monosaccharide from a glycosyl donor (usually a sugar nucleotide) to a glycosyl acceptor with high efficiency. Most enzymes operate at room temperature in aqueous solutions (pH 6-8), which makes it possible to combine several enzymes in one pot for multi-step reactions. The high regioselectivity, stereoselectivity and catalytic efficiency make enzymes especially useful for practical synthesis of oligosaccharides and glycoconjugates. See Koeller, K. M. and Wong, C.-H., Nature 2001, 409, 232-240; Wymer, N. and Toone, E. J., Curr. Opin. Chem. Biol. 2000, 4, 110-119; Gijsen, H. J. M.; Qiao, L.; Fitz, W. and Wong, C.-H., Chem. Rev. 1996, 96, 443-473.

Recent advances in isolating and cloning glycosyltransferases from mammalian and non-mammalian sources such as bacteria facilitate production of various oligosaccharides. DeAngelis, P. L., Glycobiol. 2002, 12, 9R-16R; Endo, T. and Koizumi, S., Curr. Opin. Struct. Biol. 2000, 10, 536-541; Johnson, K. F., Glycoconj. J. 1999, 16, 141-146. In general, bacterial glycosyltransferases are more relaxed regarding donor and acceptor specificities than mammalian glycosyltransferases. Moreover, bacterial enzymes are well expressed in bacterial expression systems such as E. coli that can easily be scaled up for over expression of the enzymes. Bacterial expression systems lack the post-translational modification machinery that is required for correct folding and activity of the mammalian enzymes whereas the enzymes from the bacterial sources are compatible with this system. Thus, in many embodiments, bacterial enzymes are used as synthetic tools for generating glycans, rather than enzymes from the mammalian sources.

For example, the repeating Galβ(1-4)GlcNAc-unit can be enzymatically synthesized by the concerted action of β4-galactosyltransferase (β4GalT) and β3-N-acetyllactosamninyltransferase (β3GlcNAcT). Fukuda, M., Biochim. Biophys. Acta. 1984, 780:2, 119-150; Van den Eijnden, D. H.; Koenderman, A. H. L. and Schiphorst, W. E. C. M., J. Biol. Chem. 1988, 263, 12461-12471. The inventors have previously cloned and characterized the bacterial N. meningitides enzymes β4GalT-GalE and β3GlcNAcT and demonstrated their utility in preparative synthesis of various galactosides. Blixt, O.; Brown, J.; Schur, M.; Wakarchuk, W. and Paulson, J. C., J. Org. Chem. 2001, 66, 2442-2448; Blixt, O.; van Die, I.; Norberg, T. and van den Eijnden, D. H., Glycobiol 1999, 9, 1061-1071. β4GalT-GalE is a fusion protein constructed from β4GalT and the uridine-5′-diphospho-galactose-4′-epimerase (GalE) for in situ conversion of inexpensive UDP-glucose to UDP-galactose providing a cost efficient strategy. Further examples of procedures used to generate the glycans, libraries and arrays of the invention are provided in the Examples.

In most cases, the structures of the glycans used in the compositions, libraries and arrays of the invention are described herein. However, in some cases a source of the glycan, rather than the precise structure of the glycan is given. Hence, a glycan from any available natural source can be used in the arrays and libraries of the invention. For example, known glycoproteins are a useful source of glycans. The glycans from such glycoproteins can be isolated using available procedures or, for example, procedures provided herein. Such glycan preparations can then be used in the compositions, libraries and arrays of the invention.

Examples of glycans provided in the libraries and on the arrays of the invention are provided in Table 1. Abbreviated names as well as complete names are provided.

TABLE 1

| No. | Glycan |
|---|---|
| 1. | AGP α-acid glycoprotein |
| 2. | AGPAα-acid glycoprotein glycoformA |
| 3. | AGPBα-acid glycoprotein glycoformB |
| 4. | Ceruloplasmine |
| 5. | Fibrinogen |
| 6. | Transferrin |
| 7. | (Ab4[Fa3]GNb)2#sp1 LeX |
| 8. | (Ab4[Fa3]GNb)3#sp1 LeX |
| 9. | (Ab4GNb)3#sp1 Tri-LacNAc |
| 10. | [3OSO3]Ab#sp2 3SuGal |
| 11. | [3OSO3]Ab3ANa#sp2 3′SuGalβ3GalNAc |
| 12. | [3OSO3]Ab3GNb#sp2 3′SuGalβ3GalNAc |
| 13. | [3OSO3]Ab4[6OSO3]Gb#sp1 3′6DiSuLac |
| 14. | [3OSO3]Ab4[6OSO3]Gb#sp2 3′6DiSuLac |
| 15. | [3OSO3]Ab4Gb#sp2 3′SuLac |
| 16. | [3OSO3]Ab4GNb#sp2 3′SuLacNAc |
| 17. | [4OSO3]Ab4GNb#sp2 4′SuLacNAc |
| 18. | [6OPO3]Ma#sp2 6PMan |
| 19. | [6OSO3]Ab4[6OSO3]Gb#sp2 6′6DiSuLac |
| 20. | [6OSO3]Ab4Gb#sp1 6′SuLac |
| 21. | [6OSO3]Ab4Gb#sp2 6′SuLac |
| 22. | [6OSO3]GNb#sp2 6SuGlcNAc |
| 23. | [GNb3[GNb6]GNb4]Ana#sp2 |
| 24. | [NNa3Ab]2GNb#sp2 (Sia)2GlcNAc |
| 25. | 3OSO3Ab3[Fa4]GNb#sp2 3′SuLe a |
| 26. | 3OSO3Ab4[Fa3]GNb#sp2 3′SuLe X |
| 27. | 9NAcNNa#sp2 9NAc-Neu5Ac |
| 28. | 9NAcNNa6Ab4GNb#sp2 9NAc- |
| | Neu5Ac2,6LacNAc |
| 29. | Aa#sp2 Galα |
| 30. | Aa2Ab#sp2 Galα2Gal |
| 31. | Aa3[Aa4]Ab4GNb#sp2 |
| | Galα3[Galα4]LacNAc |
| 32. | Aa3[Fa2]Ab#sp2 Galα3[Fuc]Galβ |
| 33. | Aa3Ab#sp2 Galα3Gal |
| 34. | Aa3Ab4[Fa3]GN#sp2 Galα3Le X |
| 35. | Aa3Ab4Gb#sp1 Galα3Lac |
| 36. | Aa3Ab4GN#sp2 Galα3LacNAc |
| 37. | Aa3Ab4GNb#sp2 Galα3LacNAc |
| 38. | Aa3ANa#sp2 Galα3GalNAc |
| 39. | Aa3ANb#sp2 Galα3GalNAc |
| 40. | Aa4[Fa2]Ab4GNb#sp2 |
| | Galα4[Fucα2]LacNAc |
| 41. | Aa4Ab4Gb#sp1 Galα4Lac |
| 42. | Aa4Ab4GNb#sp1 Galα4LacNAc |
| 43. | Aa4Ab4GNb#sp2 Galα4LacNAc |
| 44. | Aa4GNb#sp2 Galα4GlcNAc |
| 45. | Aa6Gb#sp2 Galα6Gal |
| 46. | Ab#sp2 Gal |
| 47. | Ab[NNa6]ANa#sp2 6Sialyl-T |
| 48. | Ab2Ab#sp2 Galβ2Gal |
| 49. | Ab3[Ab4GNb6]ANa#sp2 6LacNAc-Core2 |
| 50. | Ab3[Fa4]GNb#sp1 Le a |
| 51. | Ab3[Fa4]GNb#sp2 Le a |
| 52. | Ab3[GNb6]ANa#sp2 Core-2 |
| 53. | Ab3[NNa6]GNb4Ab4Gb#sp4 LSTc |
| 54. | Ab3[NNb6]ANa#sp2 β6Sialyl-T |
| 55. | Ab3Ab#sp2 Galβ3Gal |
| 56. | Ab3ANa#sp2 Galβ3GalNAcα |
| 57. | Ab3ANb#sp2 Galβ3GalNAcβ |
| 58. | Ab3ANb4[NNa3]Ab4Gb#sp1 GM1 |
| 59. | Ab3ANb4Ab4Gb#sp2 a-sialo-GM1 |
| 60. | Ab3GNb#sp1 LeC |
| 61. | Ab3GNb#sp2 LeC |
| 62. | Ab3GNb3Ab4Gb4b#sp4 LNT |
| 63. | Ab4[6OSO3]Gb#sp16SuLac |
| 64. | Ab4[6OSO3]Gb#sp2 6SuLac |
| 65. | Ab4[Fa3]GNb#sp1 LeX |
| 66. | Ab4[Fa3]GNb#sp2 LeX |
| 67. | Ab4ANa3[Fa2]Ab4GNb#sp2 |
| 68. | Ab4Gb#sp1 Lac |
| 69. | Ab4Gb#sp2 Lac |
| 70. | Ab4GNb#sp1 LacNAc |
| 71. | Ab4GNb#sp2 LacNAc |
| 72. | Ab4GNb3[Ab4GNb6]ANa#sp2 (LacNAc)2- |
| | Core2 |
| 73. | Ab4GNb3Ab4[Fa3]GNb3Ab4[Fa3]GNb#sp1 |
| | LacNAc-LeX-LeX |
| 74. | Ab4GNb3Ab4Gb#sp1 LNnT |
| 75. | Ab4GNb3Ab4Gb#sp2 LNnT |
| 76. | Ab4GNb3Ab4GNb#sp1 LacNAc-LacNAc |
| 77. | Ab4GNb3ANa#sp2a 3LacANcα-Core-2 |
| 78. | Ab4GNb3ANa#sp2b 3LacNAcβ-Core-2 |
| 79. | Ab4GNb6ANa#sp2 6LacANcα-Core-2 |
| 80. | ANa#sp2 Tn |
| 81. | ANa3[Fa2]Ab#sp2 A-tri |
| 82. | ANa3Ab#sp2 GalNAcα3Gal |
| 83. | ANa3Ab4GNb#sp2 GalNAcα3LacNAc |
| 84. | ANa3ANb#sp2 GalNAcα3GalNAc |
| 85. | ANa4[Fa2]Ab4GNb#sp2 |
| | GalNAcα4[Fucα2]LacNAc |
| 86. | ANb#sp2 GalNAcβ |
| 87. | ANb3[Fa2]Ab#sp2 GalNAcβ[Fucα2]Gal |
| 88. | ANb3Ana#sp2 GAlNAcβ3GalNAc |
| 89. | ANb4GNb#sp1 LacDiNAc |
| 90. | ANb4GNb#sp2 LacDiNAc |
| 91. | Fa#sp2 Fuc |
| 92. | Fa#sp3 Fuc |
| 93. | Fa2Ab#sp2 Fucα2Gal |
| 94. | Fa2Ab3[Fa4]GNb#sp2 Le b |
| 95. | Fa2Ab3Ana#sp2 H-type 3 |
| 96. | Fa2Ab3Anb3Aa#sp3 H-type3β3Gal |
| 97. | Fa2Ab3Anb3Aa4Ab4G#sp3 Globo-H |
| 98. | Fa2Ab3ANb4[NNa3]Ab4Gb#sp1 Fucosyl- |
| | GM1 |
| 99. | Fa2Ab3GNb#sp1 H-type 1 |
| 100. | Fa2Ab3GNb#sp2 H type 1 |
| 101. | Fa2Ab4[Fa3]GNb#sp1 Le Y |
| 102. | Fa2Ab4[Fa3]GNb#sp2 LeY |
| 103. | Fa2Ab4Gb#sp1 2′FLac |
| 104. | Fa2Ab4GNb#sp1 H-type 2 |
| 105. | Fa2Ab4GNb#sp2 H-type 2 |
| 106. | Fa2Ab4GNb3Ab4GNb#sp1 H-type-2- |
| | LacNAc |
| 107. | Fa2Ab4GNb3Ab4GNb3Ab4GNb#sp1 H- |
| | type2-LacNAc-LacNAc |
| 108. | Fa2GNb#sp2 Fucα2GlcNAc |
| 109. | Fa3GNb#sp2 Fucα3GlcNAc |
| 110. | Fb3GNb#sp2 Fucβ3GlcNAc |
| 111. | Fa2Ab3ANb4[NNa3]Ab4Gb#sp3 Fucosyl- |
| | GM1 |
| 112. | Ga#sp2 Galα |
| 113. | Ga4Gb#sp2 Galα4Gal |
| 114. | Gb#sp2 Galβ |
| 115. | Gb4Gb#sp2 Galβ4Gal |
| 116. | Gb6Gb#sp2 Galβ6Gal |
| 117. | GNb#sp1 GlcNAc |
| 118. | GNb#sp2 GlcNAc |
| 119. | GNb2Ab3ANa#sp2 GlcNAcβ2-Core-1 |
| 120. | GNb3[GNb6]ANa#sp2 |
| | GlcNAcβ3[GlcNAcβ6GalNAc |
| 121. | GNb3Ab#sp2 GlcNAcβ3Gal |
| 122. | GNb3Ab3ANa#sp2 GlcNAcβ3-Core1 |
| 123. | GNb3Ab4Gb#sp1 LNT-2 |
| 124. | GNb3Ab4GNb#sp1 GlcNAcβ3LacNAc |
| 125. | GNb4[GNb6]ANa#sp2 |
| | GlcNAcβ4[GlcNAcβ6]GalNAc |
| 126. | GNb4GNb4GNb4b#sp2 Chitotriose |
| 127. | GNb4MDPLys |
| 128. | GNb6ANs#sp2 GlcANcβ6GalNAc |
| 129. | G-ol-amine glucitolamine |
| 130. | GUa#sp2 Glucurinic acidα |
| 131. | GUb#sp2 Glucuronic acidβ |
| 132. | Ka3Ab3GNb#sp1 KDNα2,3-type1 |
| 133. | Ka3Ab4GNb#sp1 KDBα2,3-LacNAc |
| 134. | Ma#sp2 Mannose α |
| 135. | Ma2Ma2Ma3Ma#sp3 |
| 136. | Ma2Ma3[Ma2Ma6]Ma#sp3 |
| 137. | Ma2Ma3Ma#sp3 |
| 138. | Ma3[Ma2Ma2Ma6]Ma#sp3 |
| 139. | Ma3[Ma6]Ma#sp3 Man-3 |
| 140. | Man-5#aa Man5-aminoacid |
| 141. | Man5-9 pool Man5-9-aminoacid |
| 142. | Man-6#aa Man6-aminoacid |
| 143. | Man-7#aa Man7-aminoacid |
| 144. | Man-8#aa Man8-aminoacid |
| 145. | Man-9#aa Man9-aminoacid |
| 146. | Na8Na#sp2 Neu5Acα2,8Neu5Ac |
| 147. | Na8Na8Na#sp2 |
| | Neu5Acα2,8Neu5Acα2,5Neu5Ac |
| 148. | NJa#sp2 Neu5Gc |
| 149. | NJa3Ab3[Fa4]GNb#sp1 Neu5GcLe a |
| 150. | NJa3Ab3GbN#sp1 Neu5Gc-type1 |
| 151. | NJa3Ab4[Fa3]GNb#sp1 Neu5Gc-LeX |
| 152. | NJa3Ab4Gb#sp1 Neu5Gcα3Lactose |
| 153. | NJa3Ab4GNb#sp1 Neu5Gcα3LacNAc |
| 154. | NJa6Ab4GNb#sp1 Neu5Gcα6LacNAc |
| 155. | NJa6ANa#sp2 Neu5Gc6GalNAc (STn) |
| 156. | NNa#sp2 Neu5Ac |
| 157. | NNa3[6OSO3]Ab4GNb#sp2 |
| | 3′Sia[6′Su]LacNAc |
| 158. | NNa3[ANb4]Ab4Gb#sp1 GM2 |
| 159. | NNa3[ANb4]Ab4GNb#sp1GM2(NAc)/CT/Sda |
| 160. | NNa3[ANb4]Ab4GNb2#sp1 |
| | sp1GM2(NAc)/CT/Sda |
| 161. | NNa3{Ab4[Fa3]GN}3b#sp1 Sia3-TriLeX |
| 162. | NNa3Ab#sp2 Neu5Acα2,3Gal |
| 163. | NNa3Ab3[6OSO3]ANa#sp2 Neu5Acα3[6Su]-T |
| 164. | NNa3Ab3[Fa4]GNb#sp2 SLe a |
| 165. | NNa3Ab3[NNa6]ANa#sp2 Di-Sia-T |
| 166. | NNa3Ab3ANa#sp2 3-Sia-T |
| 167. | NNa3Ab3GNb#sp1 Neu5Acα3Type-1 |
| 168. | NNa3Ab3GNb#sp2 Neu5Acα3Type-1 |
| 169. | NNa3Ab4[6OSO3]GNb#sp23′Sia[6Su]LacNAc |
| 170. | NNa3Ab4[Fa3][6OSO3]GNb#sp2 6Su-SLeX |
| 171. | NNa3Ab4[Fa3]GNb#sp1 SLeX |
| 172. | NNa3Ab4[Fa3]GNb#sp2 SLeX |
| 173. | NNa3Ab4[Fa3]GNb3Ab#sp2 SleX penta |
| 174. | NNa3Ab4[Fa3]GNb3Ab4GNb#sp1 |
| | SLeXLacNAc |
| 175. | NNa3Ab4Gb#sp1 3′Sialyllactose |
| 176. | NNa3Ab4Gb#sp2 3′Sialyllactose |
| 177. | NNa3Ab4GNb#sp1 3′SialyllacNAc |
| 178. | NNa3Ab4GNb#sp2 3′SialyllacNAc |
| 179. | NNa3Ab4GNb3Ab4GNb#sp1 |
| | 3′SialylDiLacNAc |
| 180. | NNa3Ab4GNb3Ab4GNb3Ab4GNb#sp1 |
| | 3′Sialyl-tri-LacNAc |
| 181. | NNa3ANa#sp2 Siaα3GalNAc |
| 182. | NNa6Ab#sp2 Siaα6Gal |
| 183. | NNa6Ab4[6OSO3]]GNb#sp2 |
| | 6′Sial[6Su]LacNAc |
| 184. | NNa6Ab4Gb#sp1 6′Sia-lactose |
| 185. | NNa6Ab4Gb#sp2 6′Sia-lactose |
| 186. | NNa6Ab4GNb#sp1 6′Sia-LacNAc |
| 187. | NNa6Ab4GNb#sp2 6′Sia-LacNAc |
| 188. | NNa6Ab4GNb3Ab4[Fa3]GNb3Ab4[Fa3]GNb |
| | #sp1 6Sia-LacNAc-LeX-LeX |
| 189. | NNa6Ab4GNb3Ab4GNb#sp1 6SiaLacNAc- |
| | LacNAc |
| 190. | NNa6ANa#sp2 6SiaβGalNAc |
| 191. | NNa8NNa3[ANb4]Ab4Gb#sp1 GD2 |
| 192. | NNa8NNa3Ab4Gb#sp1 GD3 |
| 193. | NNa8NNa8NNa3[ANb4]Ab4Gb#sp1 GT2 |
| 194. | NNa8NNa8NNa3Ab4Gb#sp1 GT3 |
| 195. | NNAa3[Nna6]ANa#sp2 (Sia)2-Tn |
| 196. | NNb#sp2 Siaβ |
| 197. | NNb6Ab4GNb#sp2 6′SiaβLacNAc |
| 198. | NNb6ANa#sp2 βSTn |
| 199. | OS-11#sp2 6′sialLacNAc-biantenary glycan |
| 200. | Ra#sp2 Rhamnose |

Many of the abbreviations employed in the table are defined herein or at the website lectinity.com. The website at glycominds.com explains many of the linear abbreviations. In particular, the following abbreviations were used:

Sp1=OCH2CH2NH2;

Sp2=Sp3=OCH2CH2CH2NH2

A=Gal; AN=GalNAc; G=Glc; GN=GlcNAc;

F=Fucose; NN; Neu5Ac (sialic acid);

NJ=Neu5Gc (N-glycolylsialic acid); a=α; b=β;

Su=sulfo; T=Galβ3GalNAc (T-antigen);

Tn=GalNAc (Tn-antigen); KDN=5-OH-Sia

The glycans of the invention can have linkers, labels, linking moieties and/or other moieties attached to them. These linkers, labels, linking moieties and/or other moieties can be used to attach the glycans to a solid support, detect particular glycans in an assay, purify or otherwise manipulate the glycans. For example, the glycans of the invention can have amino moieties provided by attached alkylamine groups, amino acids, peptides, or proteins. In some embodiments, the glycans have alkylamine moieties such as —OCH₂CH₂NH₂ (called Sp1) or —OCH₂CH₂CH₂NH₂ (called Sp2 or Sp3) that have useful as linking moieties (the amine) and act as spacers or linkers.

Glycan Arrays for Detecting Breast Cancer

The arrays of the invention employ a library of characterized and well-defined glycan structures. The array has been validated with a diverse set of carbohydrate binding proteins such as plant lectins and C-type lectins, Siglecs, Galectins, Influenza Hemaglutinins and anti-carbohydrate antibodies (both from crude sera and from purified serum fractions). Further description on how to make glycan arrays useful in the practice of the invention is provided in U.S. Provisional Ser. No. 60/550,667, filed Mar. 5, 2004, and U.S. Provisional Ser. No. 60/558,598, filed Mar. 31, 2004, the contents of which are incorporated herein by reference.

The inventive libraries, arrays and methods have several advantages. One particular advantage of the invention is that the arrays and methods of the invention provide highly reproducible results.

Another advantage is that the libraries and arrays of the invention permit screening of multiple glycans in one reaction. Thus, the libraries and arrays of the invention provide large numbers and varieties of glycans. For example, the libraries and arrays of the invention have at least two, at least three, at least ten, or at least 100 glycans. In some embodiments, the libraries and arrays of the invention have about 2 to about 100,000, or about 2 to about 10,000, or about 2 to about 1,000, different glycans per array. Such large numbers of glycans permit simultaneous assay of a multitude of glycan types.

Moreover, as described herein, the present arrays have been used for successfully screening a variety of glycan binding proteins. Such experiments demonstrate that little degradation of the glycan occurs and only small amounts of glycan binding proteins are consumed during a screening assay. Hence, the arrays of the invention can be used for more than one assay. The arrays and methods of the invention provide high signal to noise ratios. The screening methods provided by the invention are fast and easy because they involve only one or a few steps. No surface modifications or blocking procedures are typically required during the assay procedures of the invention.

The composition of glycans on the arrays of the invention can be varied as needed by one of skill in the art. Many different glycoconjugates can be incorporated into the arrays of the invention including, for example, naturally occurring or synthetic glycans, glycoproteins, glycopeptides, glycolipids, bacterial and plant cell wall glycans and the like. Immobilization procedures for attaching different glycans to the arrays of the invention are readily controlled to easily permit array construction.

Spacer molecules or groups can be used to link the glycans to the arrays. Such spacer molecules or groups include fairly stable (e.g. substantially chemically inert) chains or polymers. For example, the spacer molecules or groups can be alkylene groups. One example of an alkylene group is —(CH₂)n-, where n is an integer of from 1 to 10.

Unique libraries of different glycans are attached to defined regions on the solid support of the array surface by any available procedure. In general, the arrays are made by obtaining a library of glycan molecules, attaching linking moieties to the glycans in the library, obtaining a solid support that has a surface derivatized to react with the specific linking moieties present on the glycans of the library and attaching the glycan molecules to the solid support by forming a covalent linkage between the linking moieties and the derivatized surface of the solid support.

The derivatization reagent can be attached to the solid substrate via carbon-carbon bonds using, or example, substrates having (poly)trifluorochloroethylene surfaces, or more preferably, by siloxane bonds (using, for example, glass or silicon oxide as the solid substrate). Siloxane bonds with the surface of the substrate are formed in one embodiment via reactions of derivatization reagents bearing trichlorosilyl or trialkoxysilyl groups.

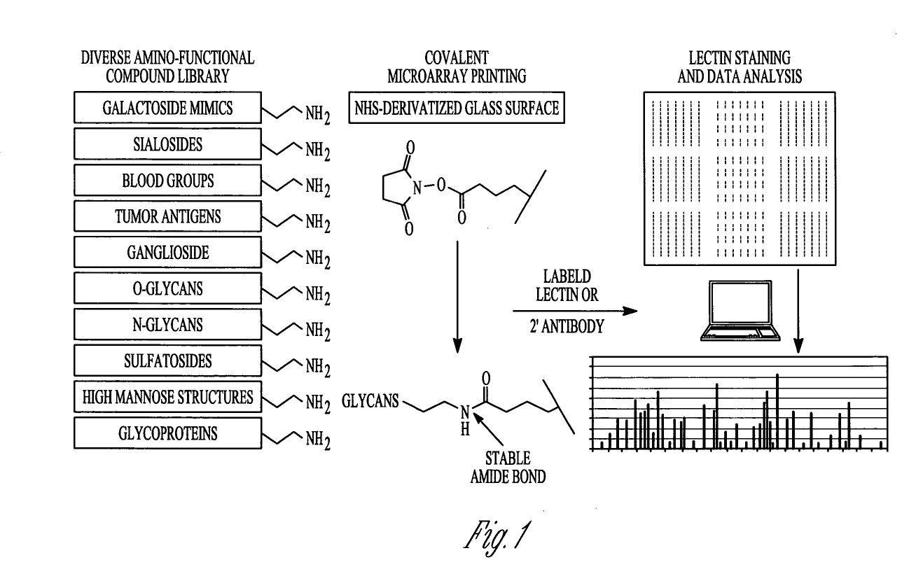

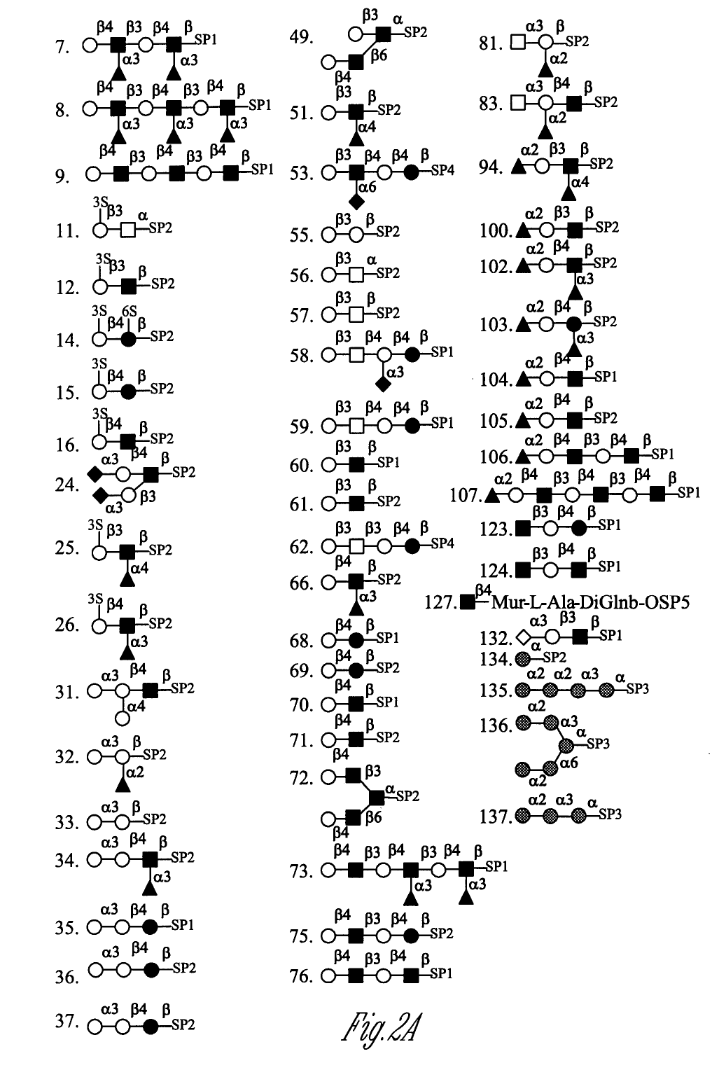

For example, a glycan library can be employed that has been modified to contain primary amino groups. For example, the glycans of the invention can have amino moieties provided by attached alkylamine groups, amino acids, peptides, or proteins. In some embodiments the glycans can have alkylamine groups such as the —OCH₂CH₂NH₂ (called Sp1) or —OCH₂CH₂CH₂NH₂ (called Sp2 or Sp3) groups attached that provide the primary amino group. The primary amino groups on the glycans can react with an N-hydroxy succinimide (NHS)-derivatized surface of the solid support. Such NHS-derivatized solid supports are commercially available. For example, NHS-activated glass slides are available from Accelr8 Technology Corporation, Denver, Colo. After attachment of all the desired glycans, slides can further be incubated with ethanolamine buffer to deactivate remaining NHS functional groups on the solid support. The array can be used without any further modification of the surface. No blocking procedures to prevent unspecific binding are typically needed. FIG. 1 provides a schematic diagram of such a method for making arrays of glycan molecules.

Each type of glycan is contacted or printed onto to the solid support at a defined glycan probe location. A microarray gene printer can be used for applying the various glycans to defined glycan probe locations. For example, about 0.1 nL to about 10 nL, or about 0.5 nL of glycan solution can be applied per defined glycan probe location. Various concentrations of the glycan solutions can be contacted or printed onto the solid support. For example, a glycan solution of about 0.1 to about 1000 micromolar glycan or about 1.0 to about 500 micromolar glycan or about 10 to about 100 micromolar glycan can be employed. In general, it may be advisable to apply each concentration to a replicate of several (for example, three to six) defined glycan probe locations. Such replicates provide internal controls that confirm whether or not a binding reaction between a glycan and a test molecule is a real binding interaction.

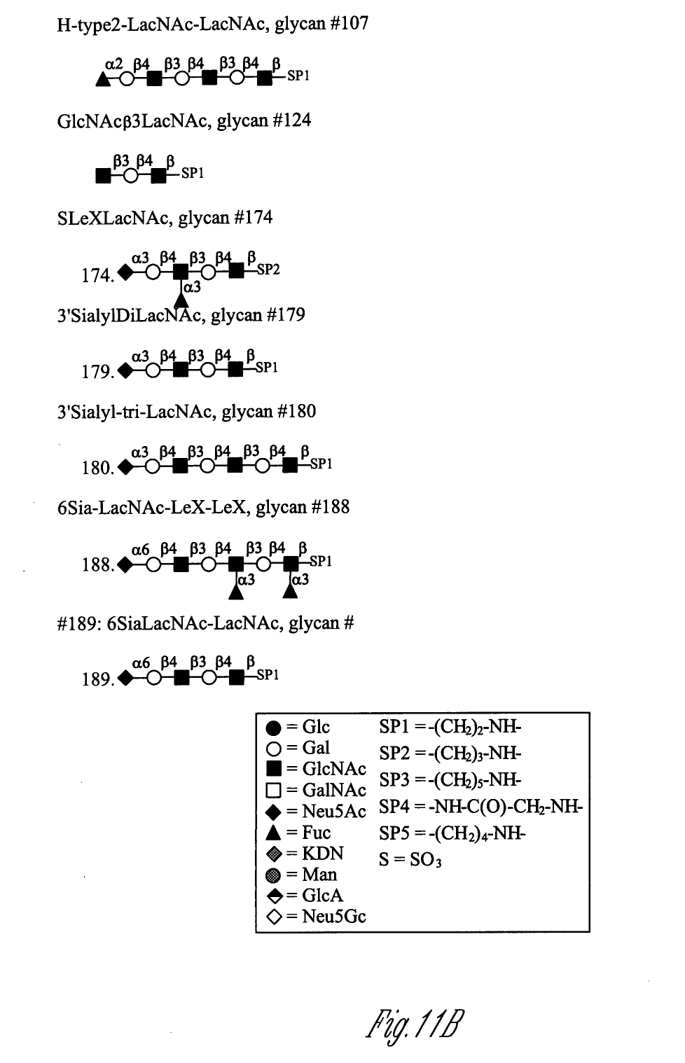

As illustrated herein, glycans that bind to antibodies in test samples from breast cancer patients include ceruloplasmin, Neu5Gc(2-6)GalNAc, GM1, Sulfo-T, Globo-H, sialylated Tn (Neu5Ac-alpha6-GalNAc-alpha) and LNT-2. Additional glycans to which antibodies from breast cancer patients bind include Circulating antibodies from breast cancer patients can also react with the following glycans: Tri-LacNAc (glycan 9 of Table 1), LacNAc-LeX-LeX (glycan 73), LacNAc-LacNAc (glycan 76), H-type-2-LacNAc (glycan 106), H-type2-LacNAc-LacNAc (glycan 107), GlcNAcβ3LacNAc (glycan 124), SLeXLacNAc (glycan 174), 3′SialylDiLacNAc (glycan 179), 3′Sialyl-tri-LacNAc (glycan 180), 6Sia-LacNAc-LeX-LeX (glycan 188), 6SiaLacNAc-LacNAc (glycan 189). The glycan numbers correspond to the glycans listed in Table 1. The structures of these glycans are shown in FIG. 11, where the linker (e.g. SP1) may be present when the glycan is used in an array. Because cancer patients have antibodies that can these glycans and the presence of such antibodies is indicative of breast cancer, many of these glycans should be present on glycan arrays used for detecting breast cancer.

Ceruloplasmin is human glycoprotein detectable in serum. Ceruloplasmin is mainly expressed and secreted by hepatocytes and is involved in copper metabolism and/or storage. See, e.g., Aouffen et al 2001, Biochem Cell Biol, 79(4), 489-97; Wang et al, Oncogene, 2002, 21, 7598-7604; Chakravarty et al., Evaluation of Ceruloplasmine concentration in prognosis of human cancer, 1986, Acta, Med, Okayama 40 (2) 103-5; Senra et al, Serum ceruloplasmine as a diagnoistic marker of cancer 1997, 121, 139-45.

Human ceruloplasmin (CAS Number 9031-37-2) can be obtained from the Sigma-Aldrich Co., St. Louis, Mo. (catalog no. C4519). The entire ceruloplasmin glycoprotein can be printed or otherwise attached to a solid support during formation of a glycan array useful for detecting breast cancer.

Other glycans to which antibodies from metastatic breast cancer patients bind include Neu5Gc(2-6)GalNAc, GM1, Sulfo-T, Globo-H, Sialylated Tn and LNT-2. Structures of these glycans are shown in FIG. 11.

Thus, GM1 has the following structure: Gal-beta3-GalNAc-beta-4-[Neu5Ac-alpha3]-Gal-beta-4-Glc-beta.

The Sulfo-T antigens are T-antigens with sulfate residues. In general, T antigens have the structure Galβ3GalNAc and the galactose sugar moieties of this glycan can have sulfate groups or other substituents.

Globo-H includes glycans with fucose-alpha2-Gal-beta3-GalNAc-beta3-Gal-alpha-4-Gal-beta-4-Glc.

The sialylated Tn glycan has the following structure: Neu5Ac-alpha6-GalNAc-alpha.

LNT-2 is a ligand for tumor-promoting Galectin-4. See Huflejt & Leffler (2004) Glycoconjugate J, 20: 247-255. The structure of LNT-2 includes the following glycan: GlcNAc-beta3-Gal-beta-4-Glc-beta.

Breast Cancer

Breast cancer usually begins in the cells lining a breast duct and in the terminal ductal lobular unit, with the first stage thought to be excessive proliferation of individual cell(s) leading to “ductal hyperplasia.” Some of the hyperplastic cells may then become atypical, with a significant risk of the atypical hyperplastic cells becoming neoplastic or cancerous. Initially, the cancerous cells remain in the breast ducts, and the condition is referred to as ductal carcinoma in situ (DCIS). After a time, however, the cancerous cells are able to invade tissues outside of the ductal environment, presenting the risk of metastases which can be fatal to the patient. Breast cancer proceeds through discrete pre-malignant and malignant cellular stages: normal ductal epithelium, atypical ductal hyperplasia, ductal carcinoma in situ (DCIS), and finally invasive ductal carcinoma. The first three stages are confined within the ductal system and, therefore, if diagnosed and treated, lead to the greatest probability of cure.

While breast cancer through the DCIS phase is in theory quite treatable, effective treatment requires both early diagnosis and an effective treatment modality. At present, mammography is the state-of-the-art diagnostic tool for detecting breast cancer. Often, however, mammography is only able to detect tumors that have reached a size in the range from 0.1 cm to 1 cm. Such a tumor mass may be reached as long as from 8 to 10 years following initiation of the disease process. Detection of breast cancer at such a late stage is often too late to permit effective treatment.

Methods of Detecting Breast Cancer

According to the invention, breast cancer patients have circulating antibodies that bind with specificity to specific types of glycans. Healthy persons who do not have breast cancer have much lower levels of such antibodies, or substantially no antibodies that react with such glycans.

Thus, in one embodiment, the invention provides methods for detecting and diagnosing breast cancer in a patient. The method involves contacting a test sample from a patient with a library or array of glycans and observing whether antibodies in the test sample bind to selected glycans. The pattern of glycans bound by antibodies from breast cancer patients can be compared to the pattern of glycans bound by antibodies in serum samples from healthy, non-cancerous patients. Glycans to which antibodies in the test sample may bind include ceruloplasmin, Neu5Gc(2-6)GalNAc, GM1, Sulfo-T, Globo-H, sialylated Tn (Neu5Ac-alpha6-GalNAc-alpha) and LNT-2. Antibodies from breast cancer patients may also bind to the following glycans: Tri-LacNAc (glycan 9 of Table 1), LacNAc-LeX-LeX (glycan 73), LacNAc-LacNAc (glycan 76), H-type-2-LacNAc (glycan 106), H-type2-LacNAc-LacNAc (glycan 107), GlcNAcβ3LacNAc (glycan 124), SLeXLacNAc (glycan 174), 3′SialylDiLacNAc (glycan 179), 3′Sialyl-tri-LacNAc (glycan 180), 6Sia-LacNAc-LeX-LeX (glycan 188), and/or 6SiaLacNAc-LacNAc (glycan 189). The glycan numbers correspond to the glycans listed in Table 1. The structures of these glycans are shown in FIG. 11, where the linker (e.g. SP1) may or may not be present on the glycan.

For detecting breast cancer, a test sample is obtained from a patient. The patient may or may not have breast cancer. In this case, the methods of the invention are used to diagnose or detect whether the patient has breast cancer or has a propensity for developing breast cancer. Alternatively, the methods of the invention can be used with patients that are known to have breast cancer. In this case, the prognosis of the breast cancer can be monitored.

The test sample obtained from the patient can be any tissue, pathology or bodily fluid sample. For example, the test sample can be is a blood sample, a serum sample, a plasma sample, a urine sample, a breast milk sample, a breast secretion sample, a nipple aspirate sample, an ascites fluid sample, a plural ascites fluid sample, a saliva sample, a cerebrospinal fluid sample, a vaginal secretion sample, an ovarian fluid sample or a tissue sample. In many embodiments, the sample is a serum sample.

Detection of binding can be direct, for example, by detection of a label attached to a molecule that binds to antibodies. Thus, detection can be indirect, for example, by detecting a labeled secondary antibody that can bind to human antibodies. The bound label can be observed using any available detection method. For example, an array scanner can be employed to detect fluorescently labeled molecules that are bound to array. In experiments illustrated herein a ScanArray 5000 (GSI Lumonics, Watertown, Mass.) confocal scanner was used. The data from such an array scanner can be analyzed by methods available in the art, for example, by using ImaGene image analysis software (BioDiscovery Inc., El Segundo, Calif.).

In general, as illustrated herein, detection of increased glycan binding by antibodies in a patient's serum is an indicator that the patient may have breast cancer. Comparison of the levels of glycan binding over time provides an indication of whether the breast cancer is progressing toward metastasis, whether a patient is responding to a selected treatment or whether the breast cancer is in remission. Hence, the invention also provides methods for monitoring the progression of breast cancer in a patient.

Further description of methods for detecting molecules that bind to glycan arrays is provided in U.S. Provisional Ser. No. 60/550,667, filed Mar. 5, 2004, and U.S. Provisional Ser. No. 60/558,598, filed Mar. 31, 2004, the contents of which are incorporated herein by reference.

Methods of Treating Breast Cancer

Conventional treatments for breast cancer have been focused on the treatment of a latter stage disease and include removal of the breast, localized removal of the tumor (“lumpectomy”), radiation, and chemotherapy. While these techniques are often effective, they suffer from certain deficiencies. Removal of the breast provides the best assurance against local recurrence of the cancer, but is disfiguring and requires the patient to make a very difficult choice. Lumpectomy is less disfiguring, but is associated with greater risk of recurrence of the cancer. Radiation and chemotherapy are arduous and are not completely effective against recurrence. Such conventional treatments therefore have drawbacks.

As described above, the invention provides methods for early detection of precancerous and cancerous conditions in the breast. In another embodiment, the invention provides compositions for preventing and treating breast cancer. Such compositions include one or more glycans that are typically recognized by circulating antibodies present in patients with metastatic breast cancer. Examples of glycans that can be included in the compositions of the invention include: ceruloplasmin, Neu5Gc(2-6)GalNAc, GM1, Sulfo-T, Globo-H, sialylated Tn (Neu5Ac-alpha6-GalNAc-alpha) and LNT-2. Further examples of glycans that can be included in the compositions of the invention include: Tri-LacNAc (glycan 9 of Table 1), LacNAc-LeX-LeX (glycan 73), LacNAc-LacNAc (glycan 76), H-type-2-LacNAc (glycan 106), H-type2-LacNAc-LacNAc (glycan 107), GlcNAcβ3LacNAc (glycan 124), SLeXLacNAc (glycan 174), 3′SialylDiLacNAc (glycan 179), 3′Sialyl-tri-LacNAc (glycan 180), 6Sia-LacNAc-LeX-LeX (glycan 188), and/or 6SiaLacNAc-LacNAc (glycan 189). The glycan numbers indicated correspond to the glycans listed in Table 1. The structures of these glycans are shown in FIG. 11, where one of skill in the art may choose to use the glycan without a spacer or linker (e.g. without SP1 or SP2) when preapring the glycan compositions of the invention.

A further aspect of the invention provides a method of treating breast cancer, the method comprising administering to the patient an effective amount of a composition that includes glycans bound by antibodies present in the serum of breast cancer patients. In some embodiments, the type and amount of glycan is effective to provoke an anti-cancer cell immune response in the patient.

The anti-breast cancer compositions of the invention may be administered directly into the patient, into the affected organ or systemically, or applied ex vivo to cells derived from the patient or a human cell line which are subsequently administered to the patient, or used in vitro to select a subpopulation from immune cells derived from the patient, which are then re-administered to the patient. The composition can be administered with an adjuvant or with immune-stimulating cytokines, such as interleukin-2. An example of an immune-stimulating adjuvant is Detox. The glycans may also be conjugated to a suitable carrier such as keyhole limpet haemocyanin (KLH) or mannan (see WO 95/18145 and Longenecker et al (1993) Ann. NY Acad. Sci. 690, 276-291). The glycans can be administered to the patient orally, intramuscularly or intradermally or subcutaneously.

In some embodiments, the compositions of the invention are administered in a manner that produces a humoral response. Thus, production of antibodies directed against the glycan(s) is one measure of whether a successful immune response has been achieved.

In other embodiments, the compositions of the invention are administered in a manner that produces a cellular immune response, resulting in tumor cell killing by NK cells or cytotoxic T cells (CTLs). Strategies of administration, which activate T helper cells are particularly useful. As described above, it may also be useful to stimulate a humoral response. It may be useful to co-administer certain cytokines to promote such a response, for example interleukin-2, interleukin-12, interleukin-6, or interleukin-10.

It may also be useful to target the immune compositions to specific cell populations, for example antigen presenting cells, either by the site of injection, by use delivery systems, or by selective purification of such a cell population from the patient and ex vivo administration of the glycan(s) to such antigen presenting cells. For example, dendritic cells may be sorted as described in Zhou et al (1995) Blood 86, 3295-3301; Roth et al (1996) Scand. J. Immunology 43, 646-651.

A further aspect of the invention therefore provides a vaccine effective against breast cancer, or against cancer or tumor cells, comprising an effective amount of glycans that are bound by circulating antibodies of metastatic breast cancer patients.

Antibodies of the Invention

The invention provides antibodies that bind to glycans that react with circulating antibodies present in metastatic breast cancer patients. Such antibodies are useful for the diagnosis, monitoring and treatment of breast cancer. As is illustrated herein, different patients may have produced different amounts and somewhat different types of antibodies against the breast-cancer associated glycan epitopes of the invention. Hence, administration of an antibodies that are known to have good affinity for the breast-cancer associated glycan epitopes of the invention will be beneficial even though the patient has begun to produce some antibodies reactive with breast cancer epitopes. Thus, the invention provides antibody preparations that can bind the breast-cancer associated glycan epitopes described herein.

Antibodies can be prepared using a selected glycan, class of glycans or mixture of glycans as the immunizing antigen. The glycan or glycan mixture can be coupled to a carrier protein, if desired. Such commonly used carrier proteins, which are chemically coupled to epitopes include keyhole limpet hemocyanin (KLH), thyroglobulin, bovine serum albumin (BSA), and tetanus toxin. A coupled protein can be used to immunize the animal (e.g., a mouse, a rat, or a rabbit).

If desired, polyclonal or monoclonal antibodies can be further purified, for example, by binding to and elution from a matrix to which the glycan or mixture of glycans to which the antibodies were raised is bound. Those of skill in the art will know of various techniques common in the immunology arts for purification and/or concentration of polyclonal antibodies, as well as monoclonal antibodies (Coligan, et al., Unit 9, Current Protocols in Immunology, Wiley Interscience, 1991, incorporated by reference).

It is also possible to use the anti-idiotype technology to produce monoclonal antibodies, which mimic an epitope. For example, an anti-idiotypic monoclonal antibody made to a first monoclonal antibody will have a binding domain in the hypervariable region, which is the “image” of the epitope bound by the first monoclonal antibody.

An antibody suitable for binding to a glycan is specific for at least one portion or region of the glycan. For example, one of skill in the art can use a whole glycan or fragment of glycan to generate appropriate antibodies of the invention. Antibodies of the invention include polyclonal antibodies, monoclonal antibodies, and fragments of polyclonal and monoclonal antibodies.

The preparation of polyclonal antibodies is well-known to those skilled in the art (Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992), which are hereby incorporated by reference). For example, a glycan or glycan mixture is injected into an animal host, preferably according to a predetermined schedule incorporating one or more booster immunizations, and the animal is bled periodically. Polyclonal antibodies specific for a glycan or glycan fragment may then be purified from such antisera by, for example, affinity chromatography using the glycan coupled to a suitable solid support.

The preparation of monoclonal antibodies likewise is conventional (Kohler & Milstein, Nature, 256:495 (1975); Coligan et al., sections 2.5.1-2.6.7; and Harlow et al., Antibodies: A Laboratory Manual, page 726 (Cold Spring Harbor Pub. 1988)), which are hereby incorporated by reference. Briefly, monoclonal antibodies can be obtained by injecting mice with a composition comprising an antigen (glycan), verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (Coligan et al., sections 2.7.1-2.7.12 and sections 2.9.1-2.9.3; Barnes et al., Purification of Immunoglobulin G (IgG), in Methods in Molecular Biology, Vol. 10, pages 79-104 (Humana Press 1992)). Methods of in vitro and in vivo multiplication of monoclonal antibodies are available to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large-scale hybridoma cultivation can be carried out by homogenous suspension culture in an air reactor, in a continuous stirrer reactor, or immobilized or entrapped cell culture. Multiplication in vivo may be carried out by injecting cell clones into mammals histocompatible with the parent cells, e.g., osyngeneic mice, to cause growth of antibody-producing tumors. Optionally, the animals are primed with a hydrocarbon, especially oils such as pristine tetramethylpentadecane prior to injection. After one to three weeks, the desired monoclonal antibody is recovered from the body fluid of the animal.

Antibodies can also be prepared through use of phage display techniques. In one example, an organism is immunized with an antigen, such as a glycan or mixture of glycans of the invention. Lymphocytes are isolated from the spleen of the immunized organism. Total RNA is isolated from the splenocytes and mRNA contained within the total RNA is reverse transcribed into complementary deoxyribonucleic acid (cDNA). The cDNA encoding the variable regions of the light and heavy chains of the immunoglobulin is amplified by polymerase chain reaction (PCR). To generate a single chain fragment variable (scFv) antibody, the light and heavy chain amplification products may be linked by splice overlap extension PCR to generate a complete sequence and ligated into a suitable vector. E. coli are then transformed with the vector encoding the scFv, and are infected with helper phage, to produce phage particles that display the antibody on their surface. Alternatively, to generate a complete antigen binding fragment (Fab), the heavy chain amplification product can be fused with a nucleic acid sequence encoding a phage coat protein, and the light chain amplification product can be cloned into a suitable vector. E. coli expressing the heavy chain fused to a phage coat protein are transformed with the vector encoding the light chain amplification product. The disulfide linkage between the light and heavy chains is established in the periplasm of E. coli. The result of this procedure is to produce an antibody library with up to 10⁹ clones. The size of the library can be increased to 10¹⁸ phage by later addition of the immune responses of additional immunized organisms that may be from the same or different hosts. Antibodies that recognize a specific antigen can be selected through panning. Briefly, an entire antibody library can be exposed to an immobilized antigen against which antibodies are desired. Phage that do not express an antibody that binds to the antigen are washed away. Phage that express the desired antibodies are immobilized on the antigen. These phage are then eluted and again amplified in E. coli. This process can be repeated to enrich the population of phage that express antibodies that specifically bind to the antigen. After phage are isolated that express an antibody that binds to an antigen, a vector containing the coding sequences for the antibody can be isolated from the phage particles and the coding sequences can be re-cloned into a suitable vector to produce an antibody in soluble form. In another example, a human phage library can be used to select for antibodies, such as monoclonal antibodies, that bind to breast cancer specific glycan epitopes. Briefly, splenocytes may be isolated from a human that has breast cancer and used to create a human phage library according to methods as described above and known in the art. These methods may be used to obtain human monoclonal antibodies that bind to the breast cancer specific glycan epitopes. Phage display methods to isolate antigens and antibodies are known in the art and have been described (Gram et al., Proc. Natl. Acad. Sci., 89:3576 (1992); Kay et al., Phage display of peptides and proteins: A laboratory manual. San Diego: Academic Press (1996); Kermani et al., Hybrid, 14:323 (1995); Schmitz et al., Placenta, 21 Suppl. A:S106 (2000); Sanna et al., Proc. Natl. Acad. Sci., 92:6439 (1995)).

An antibody of the invention may be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described (Orlandi et al., Proc. Nat'l Acad. Sci. USA, 86:3833 (1989) which is hereby incorporated in its entirety by reference). Techniques for producing humanized monoclonal antibodies are described (Jones et al., Nature, 321:522 (1986); Riechmann et al., Nature, 332:323 (1988); Verhoeyen et al, Science, 239:1534 (1988); Carter et al., Proc. Nat'l Acad. Sci. USA, 89:4285 (1992); Sandhu, Crit. Rev. Biotech., 12:437 (1992); and Singer et al., J. Immunol., 150:2844 (1993), which are hereby incorporated by reference).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens (e.g. the glycans described herein), and the mice can be used to produce human antibody-secreting hybridomas. Methods for obtaining human antibodies from transgenic mice are described (Green et al., Nature Genet., 7:13 (1994); Lonberg et al., Nature, 368:856 (1994); and Taylor et al., Int. Immunol., 6:579 (1994), which are hereby incorporated by reference).

Antibody fragments of the invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described (U.S. Pat. Nos. 4,036,945; 4,331,647; and 6,342,221, and references contained therein; Porter, Biochem. J., 73:119 (1959); Edelman et al., Methods in Enzymology, Vol. 1, page 422 (Academic Press 1967); and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments include an association of V_{H} and V_{L} chains. This association may be noncovalent (Inbar et al., Proc. Nat'l Acad. Sci. USA, 69:2659 (1972)). Alternatively, the variable chains can be linked by an intermolecular disulfide bond or cross-linked by chemicals such as glutaraldehyde (Sandhu, Crit. Rev. Biotech., 12:437 (1992)). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described (Whitlow et al., Methods: A Companion to Methods in Enzymology, Vol. 2, page 97 (1991); Bird et al., Science, 242:423 (1988), Ladner et al., U.S. Pat. No. 4,946,778; Pack et al., Bio/Technology, 11:1271 (1993); and Sandhu, Crit. Rev. Biotech., 12:437 (1992)).

Another form of an antibody fragment is a peptide that forms a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: A Companion to Methods in Enzymology, Vol. 2, page 106 (1991)).

An antibody of the invention may be coupled to a toxin. Such antibodies may be used to treat animals, including humans that suffer from breast cancer. For example, an antibody that binds to a glycan that is etiologically linked to development of breast cancer may be coupled to a tetanus toxin and administered to a patient suffering from breast cancer. The toxin-coupled antibody can bind to a breast cancer cell and kill it.

An antibody of the invention may be coupled to a detectable tag. Such antibodies may be used within diagnostic assays to determine if an animal, such as a human, has breast cancer. Examples of detectable tags include, fluorescent proteins (i.e., green fluorescent protein, red fluorescent protein, yellow fluorescent protein), fluorescent markers (i.e., fluorescein isothiocyanate, rhodamine, texas red), radiolabels (i.e., ³H, ³²P, ¹²⁵I), enzymes (i.e., â-galactosidase, horseradish peroxidase, â-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

Dosages, Formulations and Routes of Administration

The compositions of the invention are administered so as to achieve an immune response against a glycan bound by antibodies typically present in the serum of metastatic breast cancer patients. In some embodiments, the compositions of the invention are administered so as to achieve a reduction in at least one symptom associated with breast cancer.

To achieve the desired effect(s), the glycan or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, what types of glycans are administered, the route of administration, the progression or lack of progression of the breast cancer, the weight, the physical condition, the health, the age of the patient, whether prevention or treatment is to be achieved, and if the glycan is chemically modified. Such factors can be readily determined by the clinician employing animal models or other test systems that are available in the art.

Administration of the therapeutic agents (glycans) in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the glycans or combinations thereof may be essentially continuous over a preselected period of time or may be in a series of spaced doses. Both local and systemic administration is contemplated.

To prepare the composition, the glycans or antibodies or combinations thereof are synthesized or otherwise obtained, and purified as necessary or desired. These therapeutic agents can then be lyophilized or stabilized, their concentrations can be adjusted to an appropriate amount, and the therapeutic agents can optionally be combined with other agents. The absolute weight of a given glycan, binding entity, antibody or combination thereof that is included in a unit dose can vary widely. For example, about 0.01 to about 2 g, or about 0.1 to about 500 mg, of at least one glycan, binding entity, or antibody specific for a particular glycan can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the glycan(s), binding entities, antibodies or combinations thereof can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic agents of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are generally combined with a pharmaceutically acceptable carrier, diluent or excipient to form a pharmaceutical formulation, or unit dosage form. For oral administration, the therapeutic agents may be present as a powder, a granular formulation, a solution, a suspension, an emulsion or in a natural or synthetic polymer or resin for ingestion of the active ingredients from a chewing gum. The therapeutic agents may also be presented as a bolus, electuary or paste. Orally administered therapeutic agents of the invention can also be formulated for sustained release. For example, the therapeutic agents can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic agents of the invention can be prepared by procedures known in the art using well-known and readily available ingredients. For example, the therapeutic agent can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethylene glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the therapeutic agents of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one therapeutic agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more of the therapeutic agents of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic agents of the invention can also take the form of an aqueous or anhydrous solution or dispersion, or alternatively the form of an emulsion or suspension or salve.

Thus, the therapeutic agents may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The active agents and other ingredients may form suspensions, solutions, or emulsions in oily or aqueous vehicles, and may contain formulatory agents such as suspending, stabilizing and/or dispersing agents. Alternatively, the therapeutic agents and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and á-tocopherol and its derivatives can be added.

Additionally, the therapeutic agents are well suited to formulation as sustained release dosage forms and the like. The formulations can be so constituted that they release the active agent, for example, in a particular part of the vascular system or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention can be delivered via patches or bandages for dermal administration. Alternatively, the therapeutic agents can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic agents in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The active ingredients of the invention can also be administered to the respiratory tract. Thus, the present invention also provides aerosol pharmaceutical formulations and dosage forms for use in the methods of the invention.

In general, such dosage forms comprise an amount of at least one of the agents of the invention effective to treat or prevent the clinical symptoms of breast cancer. Any statistically significant attenuation of one or more symptoms of breast cancer is considered to be a treatment of breast cancer.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic agents of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the therapeutic agents of the present invention specific for the indication or disease to be treated. Dry aerosol in the form of finely divided solid therapeutic agent that are not dissolved or suspended in a liquid are also useful in the practice of the present invention. Therapeutic agents of the present invention may be formulated as dusting powders and comprise finely divided particles having an average particle size of between about 1 and 5 im, alternatively between 2 and 3 im. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular immune response, vascular condition or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, other anti-cancer agents and the like, whether for the conditions described or some other condition.

Kits

The present invention further pertains to a packaged pharmaceutical composition such as a kit or other container for detecting, controlling, preventing or treating breast cancer. In one embodiment, the kit or container holds an array or library of glycans for detecting breast cancer and instructions for using the array or library of glycans for detecting the breast cancer. The array includes at least one glycan that is bound by antibodies present in serum samples of a metastatic breast cancer patient.

In another embodiment, the kit or container holds a therapeutically effective amount of a pharmaceutical composition for controlling breast cancer and instructions for using the pharmaceutical composition for control of the breast cancer. The pharmaceutical composition includes at least one glycan of the present invention, in a therapeutically effective amount such that the breast cancer is controlled, prevented or treated.

In a further embodiment, the kit comprises a container containing an antibody that specifically binds to a glycan that is associated with breast cancer or metastatic breast cancer. The antibody can have a directly attached or indirectly associated therapeutic agent. The antibody can also be provided in liquid form, powder form or other form permitting ready administration to a patient.

The kits of the invention can also comprise containers with tools useful for administering the compositions of the invention. Such tools include syringes, swabs, catheters, antiseptic solutions and the like.

The following examples are for illustration of certain aspects of the invention and is not intended to be limiting thereof.

### EXAMPLE 1

### Enzymatic Synthesis of Glycans

The inventors have previously cloned and characterized the bacterial N. meningitides enzymes β4GalT-GalE and β3GlcNAcT. Blixt, O.; Brown, J.; Schur, M.; Wakarchuk, W. and Paulson, J. C., J. Org. Chem. 2001, 66, 2442-2448; Blixt, O.; van Die, I.; Norberg, T. and van den Eijnden, D. H., Glycobiol. 1999, 9, 1061-1071. β4GalT-GalE is a fusion protein constructed from β4GalT and the uridine-5′-diphospho-galactose-4′-epimerase (GalE) for in situ conversion of inexpensive UDP-glucose to UDP-galactose providing a cost efficient strategy.

Both enzymes, β4GalT-GalE and β3GlcNAcT, were over expressed in E. coli AD202 in a large-scale fermentor (100 L). Bacteria were cultured in 2YT medium and induced with iso-propyl-thiogalactopyranoside (IPTG) to ultimately produce 8-10 g of bacterial cell paste/L cell media. The enzymes were then released from the cells by a microfluidizer and were solubilized in Tris buffer (25 mM, pH 7.5) containing manganese chloride (10 mM) and Triton X (0.25%) to reach enzymatic activities of about 50 U/L and 115 U/L of cell culture β4GalT-GalE and β3GlcNAcT, respectively.

Specificity studies of the β3GlcNAcT (Table 2) revealed that lactose (4) is the better acceptor substrate (100%) while the enzyme shows just about 7-8% activity with N-acetyllactosamine (6). The structures of these disaccharides are provided below.

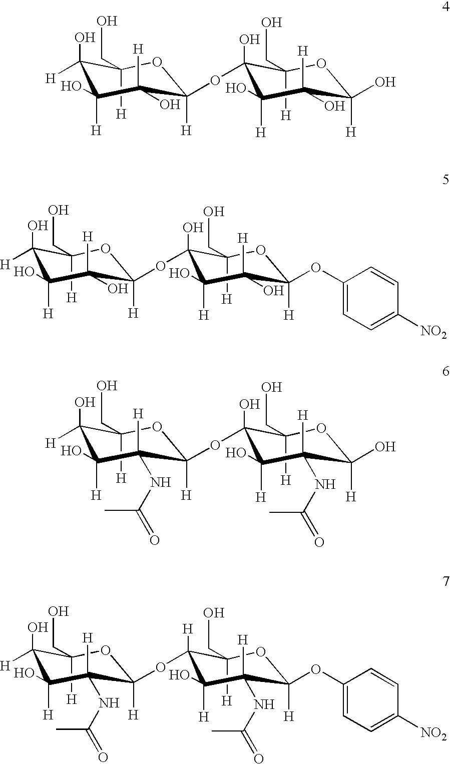

Adding the hydrophobic para-nitrophenyl ring as an aglycon to the reducing end of the acceptors enhanced the activity of the enzyme up to 10 fold (compare 4 with 5 and 6 with 7). The increase in the enzyme activity by adding a hydrophobic aglycon to the acceptor sugar, though to the lesser extent, has also been shown for β4GalT (compare 12 with 13, 14). The relaxed substrate specificity of these enzymes makes them very useful for preparative synthesis of various carbohydrate structures, including poly-N-acetyllactosamines.

TABLE 2
Selected β4GalT-GalE and β3GlcNAcT Specificity Data

| | |
|---|---|
| Acceptor | Relative enzyme activity (%) |

| | | |
|---|---|---|
| | | 1β(1-3)GlcNAcT-activity^{#} |
| 1 | Gal | 5 |
| 2 | Galα-OpNP | 102 |
| 3 | Galβ-OpNP | 16 |
| 4 | Galβ(1-4)Glc | 100 |
| 5 | Galβ(1-4)Glcβ-OpNP | 945 |
| 6 | Galβ(1-4)GlcNAc | 7 |
| 7 | Galβ(1-4)GlcNAcβ-OpNP | 74 |
| 8 | Galβ(1-3)GlcNAc | 5 |
| | | β(1-4)GalT-GalE-activity* |
| 9 | Glc | 80 |
| 10 | Glcβ-OpNP | 60 |
| 11 | GlcNH₂ | 30 |
| 12 | GlcNAc | 100 |
| 13 | GlcNAcβ-OpNP | 120 |
| 14 | GlcNAcβ-Osp₁ | 360 |
| 15 | GlcNAllocβ-sp₂ | 550 |

Abbreviations:
pNP, para-nitrophenyl;
sp_{1,} 2-azidoethyl;
sp_{2,} 5-azido-3-oxapentyl,
Alloc, allyloxycarbonyl

Poly-N-acetyllactosamine is a unique carbohydrate structure composed of N-acetyllactosamine repeats that provides the backbone structure for additional modifications, such as sialylation and/or fucosylation. These extended oligosaccharides have been shown to be involved in various biological functions by interacting as a specific ligand to selectins or galectins. Ujita, M.; McAuliffe, J.; Hindsgaul, O.; Sasaki, K.; Fukuda, M. N. and Fukuda, M., J. Biol. Chem. 1999, 274, 16717-16726; Appelmelk, B. J.; Shiberu, B.; Trinks, C.; Tapsi, N.; Zheng, P. Y.; Verboom, T.; Maaskant, J.; Hokke, C. H.; Schiphorst, W. E. C. M.; Blanchard, D.; SimoonsSmit, I. M.; vandenEijnden, D. H. and Vandenbroucke Grauls, C. M. J. E., Infect. Immun. 1998, 66, 70-76; Leppaenen, A.; Penttilae, L.; Renkonen, O.; McEver, R. P. and Cummings, R. D., J. Biol. Chem. 2002, 277, 39749-39759; Renkonen, O., Cell. Mol Life Sci. 2000, 57, 1423-1439; Baldus, S. E.; Zirbes, T. K.; Weingarten, M.; Fromm, S.; Glossmann, J.; Hanisch, F. G.; Monig, S. P.; Schroder, W.; Flucke, U.; Thiele, J.; Holscher, A. H. and Dienes, H. P., Tumor Biology. 2000, 21, 258-266; Cho, M. and Cummings, R. D., TIGG. 1997, 9, 47-56, 171-178.

Based on the specificity data in Table 2, enzymatic synthesis of galactosides and polylactosamines can be performed in multi-gram quantities. This method employed various fucosyltransferases (FUTs). Several fucosyltransferases (FUTs) have been characterized in terms of substrate specificities and biological functions in different laboratories. Murray, B. W.; Takayama, S.; Schultz, J. and Wong, C. H., Biochem. 1996, 35, 11183-11195; Weston, B. W.; Nair, R. P.; Larsen, R. D. and Lowe, J. B., J. Biol. Chem. 1992, 267, 4152-4160; Kimura, H.; Shinya, N.; Nishihara, S.; Kaneko, M.; Irimura, T. and Narimatsu, H., Biochem. Biophys. Res. Comm. 1997, 237, 131-137; Chandrasekaran, E. V.; Jain, R. K.; Larsen, R. D.; Wlasichuk, K. and Matta, K. L., Biochem. 1996, 35, 8914-8924; Devries, T.; Vandeneijnden, D. H.; Schultz, J. and Oneill, R., FEBS Lett. 1993, 330, 243-248; Devries, T. and van den Eijnden, D. H., Biochem. 1994, 33, 9937-9944

The available specificity data in combination with large scale production of recombinant FUTs made it possible to synthesize various precious fucosides in multi-gram quantities. Scheme I illustrates the general procedure employed for elongating the poly-LacNAc backbone and selected fucosylated structures using different FUTs and GDP-fucose.

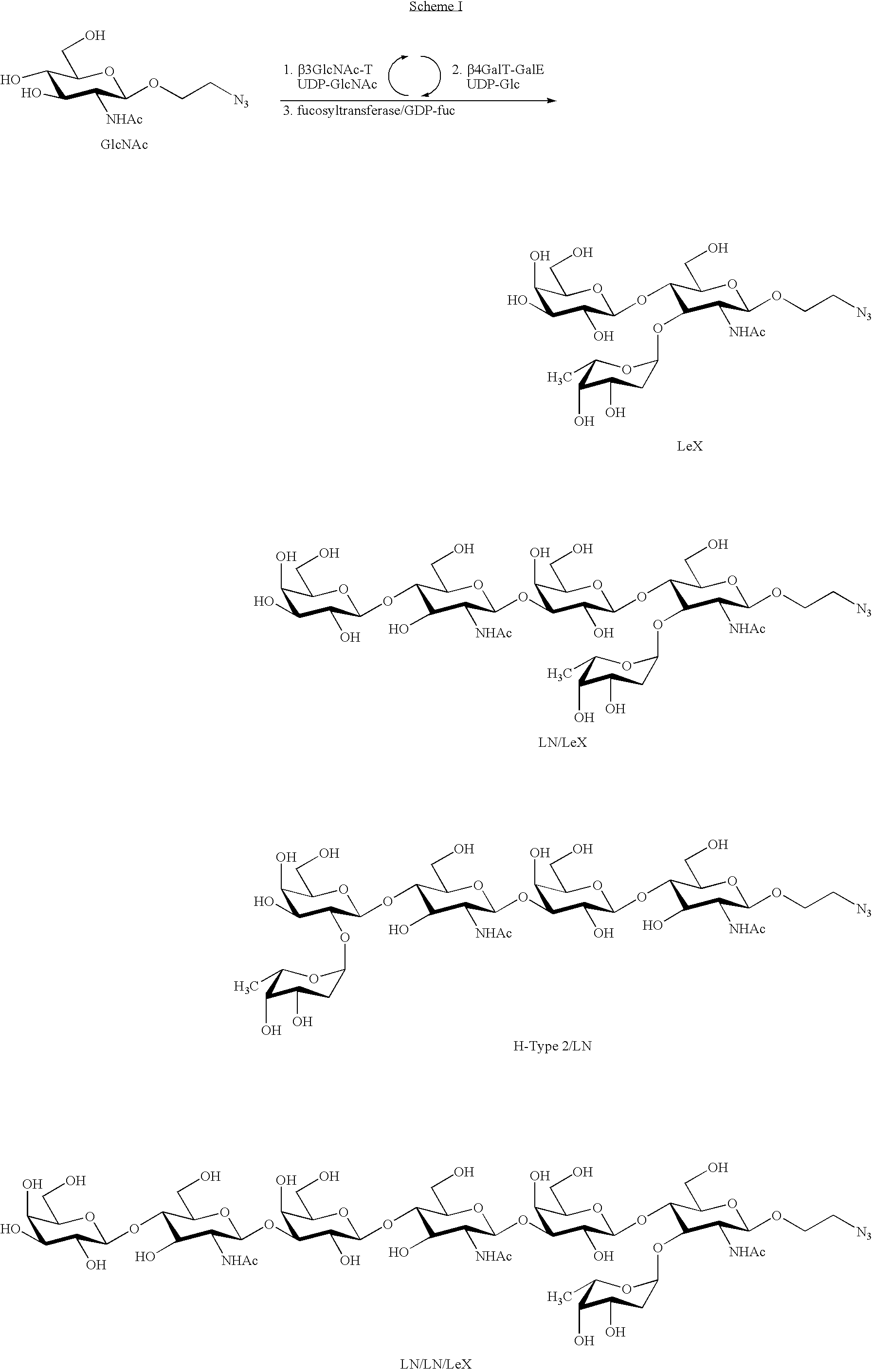

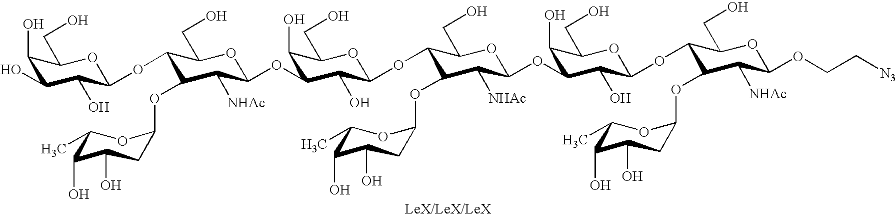

A systematic gram-scale synthesis of different fucosylated lactosamine derivatives was initiated using the Scheme I and the following recombinant fucosyltransferases, FUT-II, FUT-III, FUT-IV, FUT-V, and FUT-VI. All the above fucosyltransferases, except for FUT-V, were produced in the insect cell expression system and either partially purified on a GDP-sepharose affinity column or concentrated in a Tangential Flow Filtrator (TFF-MWCO 10k) as a crude enzyme mixture. The FUT-V enzyme was expressed in A. niger as described in Murray, B. W.; Takayama, S.; Schultz, J. and Wong, C. H., Biochem. 1996, 35, 11183-11195.

The yields for different stages of production of the fucosylated lactosamine derivatives were 75-90% for LeX (2 enzymatic steps), 45-50% for dimeric LacNAc structures (4 enzymatic steps) and 30-35% for trimeric lacNAc structures (6 enzymatic steps).

### EXAMPLE 2

### Synthesis of Sialic-Acid-Containing Oligosaccharides

Sialic acid is a generic designation used for 2-keto-3-deoxy-nonulosonic acids. The most commonly occurring derivatives of this series of monosaccharides are those derived from N-acetylneuraminic acid (Neu5Ac), N-glycolylneuraminic acid (Neu5Gc) and the non-aminated 3-deoxy-D-glycero-D-galacto-2-nonulosonic acid (KDN). Sialic-acid-containing oligosaccharides are an important category of carbohydrates that are involved in different biological regulations and functions. Sialic acids are shown to be involved in adsorption of toxins/viruses, and diverse cellular communications through interactions with carbohydrate binding proteins (CBPs). Selectins and Siglecs (sialic acid-binding immunoglobulin-superfamily lectins) are among those well-characterized CBPs that function biologically through sialic acid interactions.

Synthesis of oligosaccharides containing sialic acids is not trivial. Unfortunately, the chemical approaches have several hampering factors in common. For example, stereo selective glycosylation with sialic acid generally gives an isomeric product, and as a result, purification problems and lower yields. Its complicated nature, also require extensive protecting group manipulations and careful design of both acceptor and donor substrates and substantial amounts of efforts are needed to prepare these building blocks.

For a fast and efficient way to sialylate carbohydrate structures, the method of choice is through catalysis by sialyltransferases. Enzymatic sialylation generating Neu5Ac-containing oligosaccharides is way to generate sialylate carbohydrates for both analytical and preparative purposes. Koeller, K. M. and Wong, C.-H., Nature 2001, 409, 232-240; Gilbert, M.; Bayer, R.; Cunningham, A.-M.; DeFrees, S.; Gao, Y.; Watson, D. C.; Young, N. M. and Wakarchuk, W. W., Nature Biotechnol. 1998, 16, 769-772; Ichikawa, Y.; Look, G. C. and Wong, C. H., Anal. Biochem. 1992, 202, 215-238. However, efficient methods for preparation of oligosaccharides having the Neu5Gc or KDN structures have not previously been explored to the same extent because of the scarcity of these sialoside derivatives.

A simple way to obtain different sialoside derivatives was devised using a modification of a method, originally developed by Wong and co-workers. Crocker, P. R., Curr. Opin. Struct. Biol. 2002, 12, 609-615. This method employed recombinant sialyltransferases along with a commercial Neu5Ac aldolase, ST3-CMP-Neu5Ac synthetase. Gilbert, M.; Bayer, R.; Cunningham, A.-M.; DeFrees, S.; Gao, Y.; Watson, D. C.; Young, N. M. and Wakarchuk, W. W., Nature Biotechnol. 1998, 16, 769-772.

The preferred route to generate Neu5Ac-oligosaccharides was to use a one-pot procedure described in Scheme II (B and C).

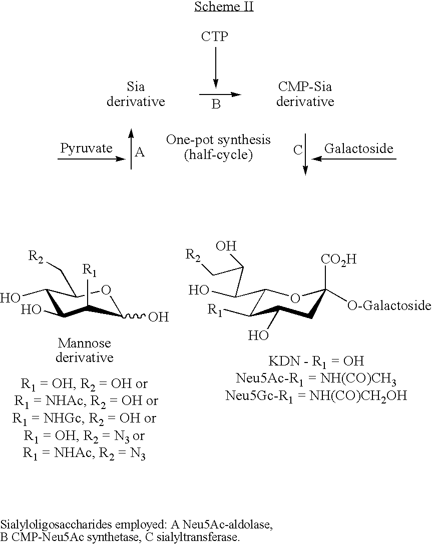

Briefly, ST3-CMP-Neu5Ac synthetase catalyzed the formation of CMP-Neu5Ac quantitatively from 1 equivalent of Neu5Ac and 1 equivalent of CTP. After removal of the fusion protein by membrane filtration (MW cut-off 10k) a selected galactoside and a recombinant sialyltransferase as described in Table 3 was introduced to produce the desired Neu5Ac-sialoside.

TABLE 3
Recombinant Sialyltransferases Produced for Synthesis

| Sialyltransferase | Source of Production | Produced Activity* |
|---|---|---|
| hST6Gal-I | Baculovirus (19) | 20 |
| pST3Gal-I | Baculovirus (45) | 20 |
| rST3Gal-III | A. Niger^{#} | 50 |
| chST6Gal-I | Baculovirus (46) | 10 |
| ST3Gal-Fusion | E. coli (42) | 6000 |
| ST8 (Cst-II) | E. coli (70) | 140 |

*Units/L cell culture

This synthetic scheme produced multi-gram quantities of product typically with a yield of 70-90% recovery of sialylated products.

To synthesize Neu5Gc and KDN derivatives the one-pot system would include another enzymatic reaction in addition to routes B and C (Scheme II). In this respect, mannose derivatives, pyruvate (3 eqv.) and commercial microorganism Neu5Ac aldolase (Toyobo) were introduced into the one-pot half-cycle (Scheme II, A). The enzymes in Table 3 were able to generate various N- and O-linked oligosaccharides with α(2-3)-, α(2-6)- or α(2-8)-linked sialic acid derivatives of Neu5Gc, KDN and some of the 9-azido-9deoxy-Neu5Ac-analogs in acceptable yields (45-90%). O-linked sialyl-oligosaccharides are another class of desired compounds for the biomedical community. These structures are frequently found in various cancer tissues and lymphoma and are highly expressed in many types of human malignancies including colon, breast, pancreas, ovary, stomach, and lung adenocarcinomas. Dabelsteen, E., J. Pathol. 1996, 179, 358-369; Itzkowitz, S. H.; Yuan, M.; Montgomery, C. K.; Kjeldsen, T.; Takahashi, H. K. and Bigbee, W. L., Cancer Res. 1989, 49, 197-204.

The inventors have previously reported the cloning, expression, and characterization of chicken ST6GalNAc-I and its use in preparative synthesis of the O-linked sialoside antigens, STn-, α(2-6)SiaT-, α(2-3)SiaT- and Di-SiaT-antigen. Blixt, O.; Allin, K.; Pereira, L.; Datta, A. and Paulson, J. C., J. Am. Chem. Soc. 2002, 124, 5739-5746. Briefly, the recombinant enzyme was expressed in insect cells and purified by CDP-sepharose affinity chromatography to generate approximately 10 U/L of cell culture. The enzymatic activity was evaluated on a set of small acceptor molecules (Table 4), and it was found that an absolute requirement for enzymatic activity is that the anomeric position on GalNAc is α-linked to threonine.

TABLE 4

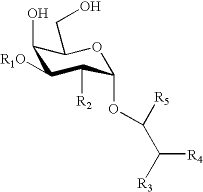

chST6GalNAc-I Activity of α-D-Galacto Derivatives

| Compound | R₁ | R₂ | R₃ | R₄ | R₅ | cpm | nmol/mg x min⁻¹ |
|---|---|---|---|---|---|---|---|
| D-GalNAc | H | NHAc | | | | 0 | 0.00 |
| 1 | H | NHAc | N₃ | H | H | 65 | 0.06 |
| 2 | H | NHAc | NHAc | H | H | 121 | 0.11 |
| 3c | H | NHAc | NHAc | COOCH₃ | CH₃ | 9133 | 8.60 |
| 4 | H | N₃ | NHAc | COOCH₃ | CH₃ | 3043 | 2.90 |
| 5 | H | NH₂ | NHAc | COOCH₃ | CH₃ | 1421 | 1.30 |
| 6 | H | NHAc | NHF_{moc} | COOCH₃ | CH₃ | 13277 | 12.50* |
| 7c | Galβ1,3 | NHAc | NHAc | COOCH₃ | CH₃ | 12760 | 12.00 |

NOTE:
*Product was isolated by using Sep-Pak (C18) cartridges as described in Palcic, M. M.; Heerze, L. D.; Pierce, M. and Hindsgaul, O., Glycoconj. J. 1988, 5, 49-63.

Thus, O-linked sialosides terminating with a protected threonine could successfully be synthesized on gram-scale reactions using Scheme IV. To be able to attach these compounds to other functional groups, the N-acetyl protecting group on threonine could be substituted with a removable 9-fluorenyl (F-moc) derivative before enzymatic extension with chST6GalNAc-I. Blixt, O.; Collins, B. E.; Van Den Nieuwenhof, I. M.; Crocker, P. R. and Paulson, J. C., (2003 J. Biol. Chem. 15: 278). As seen in Table 4, the enzyme was not sensitive to bulky groups at this position (compound 6).

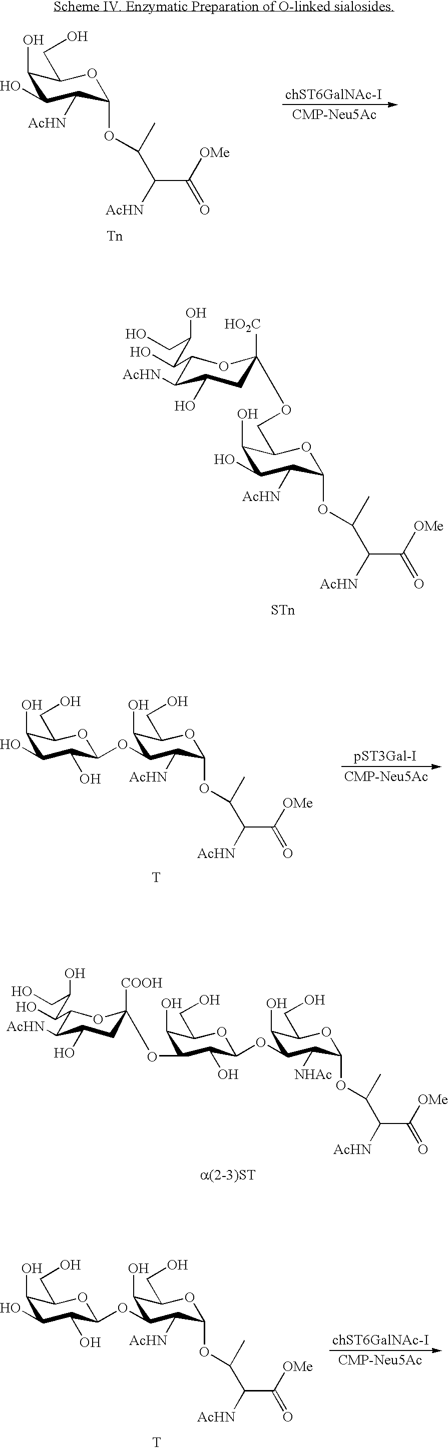

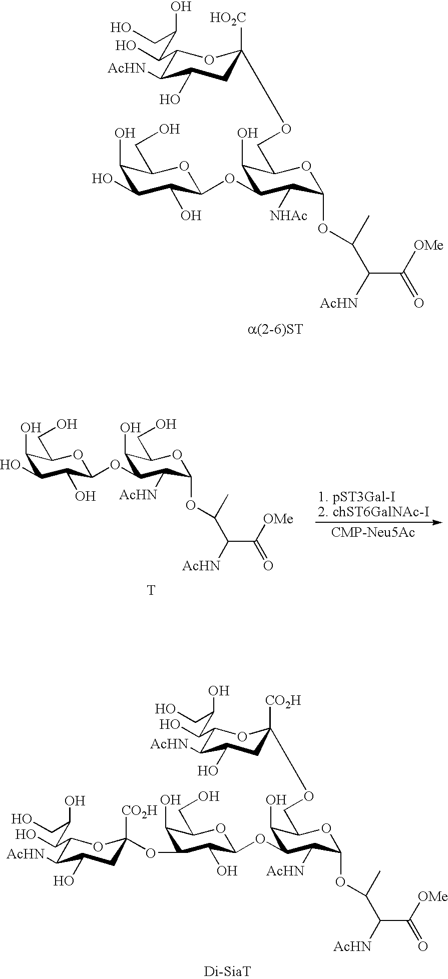

### EXAMPLE 3

### Synthesis of Ganglioside Mimics

Gangliosides are glycolipids that comprise a structurally diverse set of sialylated molecules. They are attached and enriched in nervous tissues and they have been found to act as receptors for growth factors, toxins and viruses and to facilitate the attachment of human melanoma and neuroblastoma cells. Kiso, M., Nippon Nogei Kagaku Kaishi. 2002, 76, 1158-1167; Gagnon, M. and Saragovi, H. U., Expert Opinion on Therapeutic Patents. 2002, 12, 1215-1223; Svennerholm, L., Adv. Gen. 2001, 44, 33-41; Schnaar, R. L., Carbohydr. Chem. Biol. 2000, 4, 1013-1027; Ravindranath, M. H.; Gonzales, A. M.; Nishimoto, K.; Tam, W.-Y.; Soh, D. and Morton, D. L., Ind. J. Exp. Biol. 2000, 38, 301-312; Rampersaud, A. A.; Oblinger, J. L.; Ponnappan, R. K.; Burry, R. W. and Yates, A. J., Biochem. Soc. Trans. 1999, 27, 415-422; Nohara, K., Seikagaku. 1999, 71, 337-341.

Despite the importance of these sialylated ganglioside structures, methods for their efficient preparation have been limiting. The introduction of sialic acid to a glycolipid core structure have shown to be a daunting task, needed complicated engineering with well executed synthetic strategies.

Recently, several glycosyltransferase genes from Campylobacter jejuni (OH4384) have been identified to be involved in producing various ganglioside-related lipoligosaccharides (LOS) expressed by this pathogenic bacteria. Gilbert, M.; Brisson, J.-R.; Karwaski, M.-F.; Michniewicz, J.; Cunningham, A.-M.; Wu, Y.; Young, N. M. and Wakarchuk, W. W., J. Biol. Chem. 2000, 275, 3896-3906. Among these genes, cst-II, coding for a bifunctional a(2-3/8) sialyltransferase, has been demonstrated to catalyze transfers of Neu5Ac a(2-3) and a(2-8) to lactose and sialyllactose, respectively. Another gene, cgtA, coding for a β(1-4)-N-acetylgalactosaminyltransferase (β4GalNAcT) that is reported to transfer GalNAc β(1-4) to Neu5Acα(2-3)lactose acceptors generating the GM2 (Neu5Acα(2-3)[GalNAcβ(1-4)]Galβ(1-4)Glc-) epitope.

The gene products of the two glycosyltransferase genes (cst-II and cgtA) were successfully over expressed in large scale (100 L E. coli fermentation) and used in the preparative synthesis of various ganglioside mimics. For synthetic purposes an extensive specificity study of these enzymes was also conducted using neutral and sialylated structures to further specify the synthetic utility of these enzymes.

For a cost-efficient synthesis of GalNAc-containing oligosaccharides, expensive uridine-5′-diphosphate-N-acetylgalactosamine (UDP-GalNAc) was produced in situ from inexpensive UDP-GlcNAc by the UDP-GlcNAc-4′-epimerase (GalNAc-E). GalNAc-E was cloned from rat liver into the E. coli expression vector (pCWori) and expressed in E. coli AD202 cells. Briefly, a lactose derivative was elongated with sialic acid repeats using a(2-8)-sialyltransferase and crude CMP-Neu5Ac. Several products (GM3, GD3, GT3) were isolated from this mixture. Increasing CDP-Neu5Ac from 2.5 to 4 equivalents favors the formation of GT3, and minor amounts of GD3 were isolated. Typical yields range from 40-50% of the major compound and 15-20% for the minor compound. Isolated compounds were further furbished with the action of GM2-synthetase (CgtA) and GalE to give the corresponding GM2, GD2, and GT2 structures in quantitative yields (Scheme V).

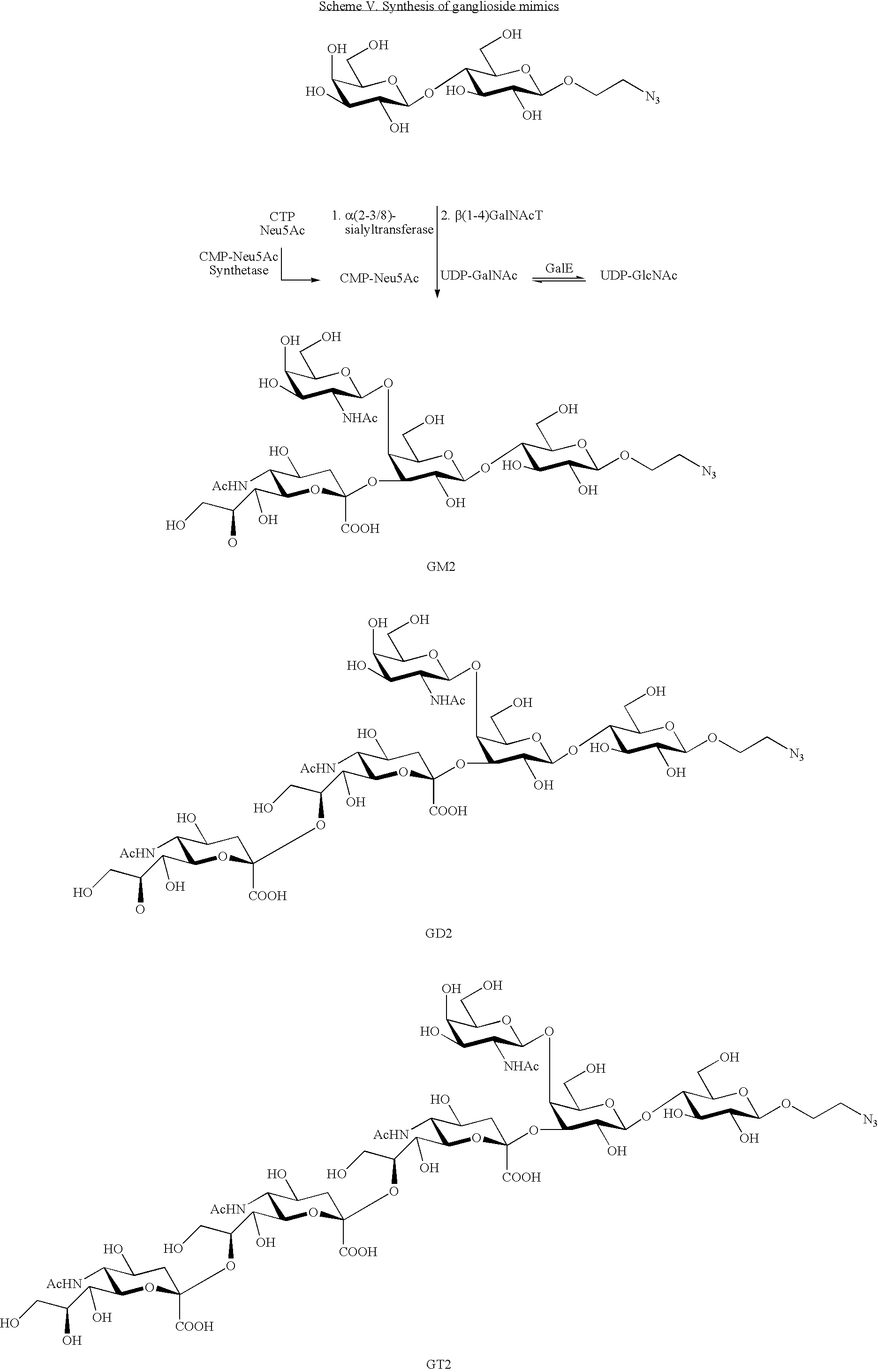

Therefore, methodologies were developed for generating diverse series of glycans, such as poly-N-acetyllactosamine and its corresponding fucosylated and/or sialylated compounds, various sialoside derivatives of N- and O-linked glycans, and ganglioside mimic structures. Furthermore, a simple route to produce the scarce sialic acid derivatives was described. This work demonstrates that chemoenzymatic synthesis of complicated carbohydrate structures can reach a facile and practical level by employing a functional toolbox of different glycosyltransferases. Detailed information of the specificity of these enzymes is needed for developing a library of glycan compounds with an extensive structural assortment. The invention provides such a library of carbohydrates and methods for using the library in high throughput studies of carbohydrate-protein, as well as, carbohydrate-carbohydrate interactions.

### EXAMPLE 4

### Isolating Glycans from Natural Sources

The Example illustrates how certain type of mannose-containing glycans can be isolate from bovine pancreatic ribonuclease B.

Pronase Digestion of Bovine Pancreatic Ribonuclease B: Bovine pancreatic ribonuclease B (Sigma Lot 060K7650) was dissolved in buffer (0.1M Tris+1 mM MgCl₂+1 mM CaCl₂ pH 8.0) and pronase (Calbiochem Lot B 50874) was added to give a ratio by weight of five parts glycoprotein to one part pronase. It was incubated at 60° c. for 3 hours. Mannose-containing glycans in the digested sample were affinity purified using a freshly prepared Con A in buffer (0.1M Tris, 1 mM MgCl₂, 1 mM CaCl₂, pH 8.0), washed and eluted with 200 mls 0.1M methyl-a-D-mannopyranoside (Calbiochem Lot B37526). The Con A eluted sample was purified on Carbograph solid-phase extraction column (Alltech 1000 mg, 15 ml) and eluted with 30% acetonitrile+0.06% TFA. It was dried and reconstituted in 1 ml water. Mass analysis was done by MALDI and glycan quantification by phenol sulfuric acid assay.

The pronase digested ribonuclease b was diluted with 5 mls 0.1M Tris pH 8.0 loaded onto 15 mls Con A column in 0.1M Tris, 1 mM MgCl₂, 1 mM CaCl₂, pH 8.0, washed and eluted with 50 mls 0.1M methyl-á-D mannopyranoside. It was then purified on Carbograph solid-phase extraction column (Alltech 1000 mg, 15 ml) eluted with 80% acetonitrile, containing 0.1% TFA, dried and reconstituted in 2 ml water. Mass analysis and glycan quantification were performed using a Voyager Elite MALDI-TOF (Perseptive BioSystems) in negative mode.

Separation of Fractions on Dionex: Pronase digested ribonuclease b was injected on the DIONEX using a PA-100 column and eluted with the following gradient: Solution A=0.1M NaOH, B=0.5M NaOAc in 0.1M NaOH; 0% B for 3 mins, then a linear gradient from 0% B to 6.7% B in 34 mins. The individual peak fractions were collected and purified on Carbograph solid-phase columns (Alltech 150 mg, 4 ml) by eluting with 80% acetonitrile containing 0.1% TFA. They were dried and reconstituted in water. Final Mass analysis and glycan quantification were performed.

### EXAMPLE 5

### Preparation and Use of Glycan Arrays

This Example illustrates some of the procedures used for making and using the glycan arrays of the inventions. Some of the subject matter described in this Example has previously been described in U.S. Provisional Ser. No. 60/550,667, filed Mar. 5, 2004, and U.S. Provisional Ser. No. 60/558,598, filed Mar. 31, 2004, the contents of which are incorporated herein by reference.

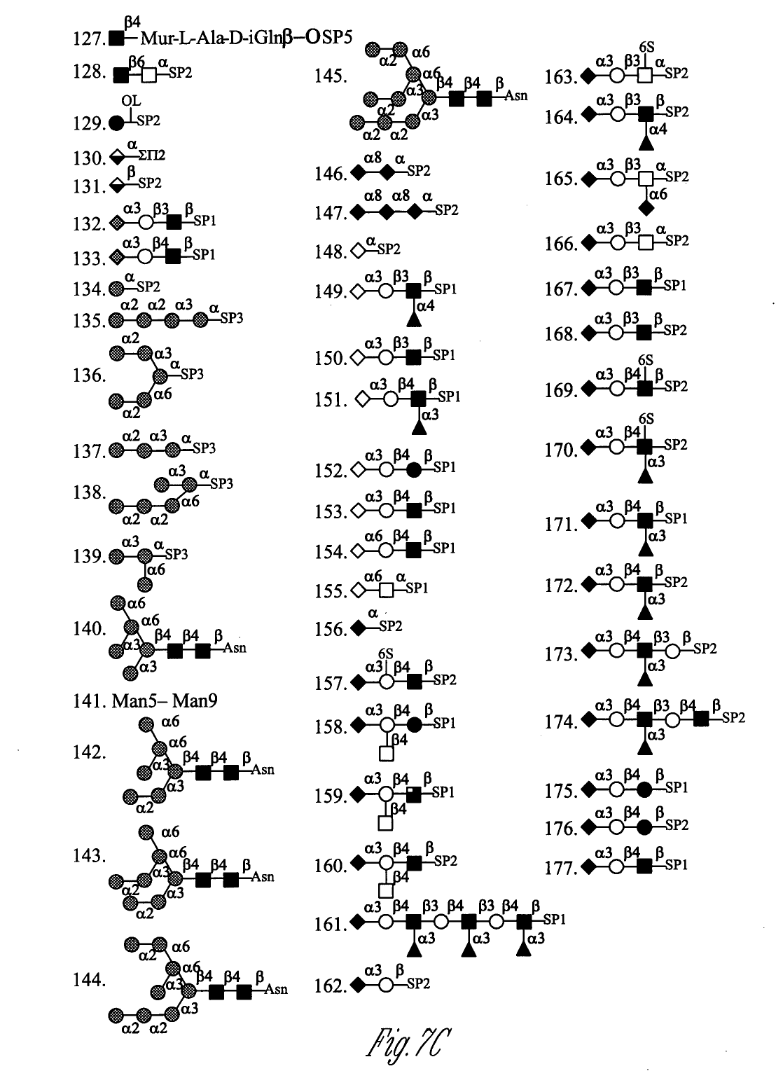

Materials. Natural glycoproteins, alpha1-acid glycoprotein (α₁-AGP), α₁-AGP glycoform A and B were prepared as described in Shiyan, S. D. & Bovin, N. V. (1997) Glycoconj. J 14, 631-8. Ceruloplasmin, fibrinogen, and apo-transferrin were obtained from Sigma-Aldrich Chemical Company, MO. Synthetic glycan ligands 7-134, 146-200 (structures shown in FIG. 7) were from The Consortium for Functional Glycomics or prepared as described in Pazynina et al. (2003) Mendeleev Commun. 13, 245-248; Pazynina et al. (2002) Mendeleev Commun. 12, 183-184; Pazynina et al. (2002) Tet. Lett. 43, 8011-8013; Nifant'ev et al. (1996) J. Carbohydr. Chem. 15, 939-953; Zemlyanukhina et al. (1995) Carbohydr. Lett. 1, 277-284. Ligands 111, 135-139 (shown in FIG. 7) were obtained through one-pot chemical synthesis as described in Lee et al. (2004) Angew. Chem. Int. Ed. 43, 1000-1003. Ligands 140-145 (shown in FIG. 7) were isolated from ribonuclease as described in supplementary information.

NHS-activated glass slides (Slide-H) were from Schott Nexterion (Germany) and the robotic printing arrayer was custom made by Robotic Labware Designs (Carlsbad, Calif.). Arrays were printed using CMP4B microarray spotting pins (TeleChem International, Inc).

Several glycan binding proteins (GBPs) were obtained from commercial sources (Con A and ECA from EY-laboratories Inc., San Mateo, Calif.; anti-CD15 from BD Biosciences, San Jose, Calif.). Other types of glycan binding proteins were obtained from various investigators including DC-SIGN (van Die et al. (2003) Glycobiology 13, 471-478), Influenza virus, A/Puerto Rico/8/34 (H1N1) (Gamblin et al. (2004) Science 303, 1838-42), 2G12 (Calarese et al. (2003) Science 300, 2065-71), Cyanovirin-N (Scanlan et al. (2002) J. Virol. 76, 7306-21), H3 HA (Stevens, Blixt and Wilson; manuscript in preparation).

Human serum was obtained from healthy volunteers at The General Clinical Research Center, Scripps Hospital, La Jolla. The samples were centrifuged for 30 min at 3000 rpm and heat inactivated at 56° C. for 25 minutes. CD22 was expressed and purified as described in Blixt et al. (2003) J. Biol. Chem. 278, 31007-19. Recombinant human Galectin-4 was also prepared as described for rat Galectin-4 by Huflejt et al. (1997) J. Biol. Chem. 272, 14294-303. Galectin-4-AlexaFluor488 was made with AlexaFluor488 protein labeling Kit from Molecular Probes according to the manufacturer's instructions. Rabbit anti-CVN was obtained as described in Scanlan et al. (2002) J. Virol. 76, 7306-21. Monoclonal mouse anti-human-IgG-IgM-IgA-Biotin antibody and Streptavidin-FITC were from Pierce, Rockford, Ill. Rabbit anti-goat-IgG-FITC, goat anti-human-IgG-FITC, mouse anti-HisTag-IgG-Alexafluor-488 and anti-mouse-IgG-Alexafluor-488 were purchased from Vector Labs (Burlingame, Calif.). Rabbit anti-Influenza virus A/PR/8/34 was from the World Influenza Centre, Mill Hill, London, UK. Other reagents and consumables were from commercial sources with highest possible quality.

Pronase Digestion of Bovine Pancreatic Ribonuclease B. 540 mg of bovine pancreatic ribonuclease b (Sigma Lot 060K7650) was dissolved in 5 mls of 0.1M Tris+1 mM MgCl₂+1 mM CaCl₂ pH 8.0. 108 mg of pronase (Calbiochem Lot B 50874) was added to give a ratio by weight of five parts glycoprotein to one part pronase. This mixture was incubated at 60° C. for 3 hours. A second dose of 108 mg pronase was added and incubated at 37° C. for another 3 hours, after which it was boiled for 30 minutes, cooled and centrifuged. The sample was loaded onto 20 ml of freshly prepared ConA in 0.1M Tris, 1 mM MgCl₂, 1 mM CaCl₂, pH 8.0, washed and eluted with 200 ml 0.1M methyl-á-D-mannopyranoside (Calbiochem Lot B37526). The Con A eluted sample was purified on Carbograph solid-phase extraction column (Alltech 1000 mg, 15 ml) and eluted with 30% acetonitrile+0.06% TFA. The eluate was dried and reconstituted in 1 ml water. Mass analysis was done by MALDI and glycan quantification by phenol sulfuric acid assay.

Carbohydrates obtained from bovine pancreatic ribonuclease B were separated by DIONEX chromatography. 20 ul of the pronase digested ribonuclease b was injected on the DIONEX using a PA-100 column and eluted with the following gradient (solution A=0.1M NaOH, solution B=0.5M NaOAc in 0.1M NaOH): 0% B for 3 min, then a linear gradient from 0% B to 6.7% B for 34 min. The individual peak fractions were collected and purified on Carbograph solid-phase columns (Alitech 150 mg, 4 ml) by elution with 80% acetonitrile containing 0.1% TFA. The peak fractions were then dried and reconstituted in water. Final Mass analysis and glycan quantification were performed.

Glycan array fabrication. Microarrays were printed by robotic pin deposition of ˜0.6 nL of various concentrations (10-100 μM) of amine-containing glycans in print buffer (300 mM phosphate, pH 8.5 containing 0.005% Tween-20) onto NHS-activated glass slides. Each compound was printed at two concentrations (100 μM and 10 μM) and each concentration in a replicate of six. Printed slides were allowed to react in an atmosphere of 80% humidity for 30 min followed by desiccation over night. Remaining NHS-groups were blocked by immersion in buffer (50 mM ethanolamine in 50 mM borate buffer, pH 9.2) for 1 hr. Slides were rinsed with water, dried and stored in desiccators at room temperature prior to use.

Glycan Binding Protein binding assay. Printed slides were analyzed without any further modification of the surface. Slides were incubated in either a one step procedure with labeled proteins, or a sandwich procedure in which the bound glycan binding protein (GBP) was overlaid with labeled secondary antibodies or GBP's pre-complexed with labeled antibodies. GBP's were added at a concentration of 5-50 μg/mL in buffer (usually PBS containing 0.005-0.5% Tween-20). Secondary antibodies (10 μg/mL in PBS) were overlaid on bound GBP. GBP-antibody pre-complexes were prepared in a molar ratio of 1:0.5:0.25 (5-50 μg/mL) for GBP:2° antibody:3° antibody, respectively (15 min on ice). The samples (50-100 μL) were applied either directly onto the surface of a single slide and covered with a microscope cover slip, or applied between two parallel slides separated by thin tape and pressed together by paper clips (see Ting et al. (2003) BioTechniques 35, 808-810) and then incubated in a humidified chamber for 30-60 minutes. Slides were subsequently washed by successive rinses in (i) PBS-Tween (0.05%), (ii) PBS and (iii) de-ionized water, then immediately subjected to imaging. Serum samples were typically used at dilutions of 1:25 and 0.4-0.8 mL applied directly onto the slide surface without any cover glass. The slides were gently rocked at room temperature for 90 min followed by detection with secondary antibodies (Table 6). Whole virus was applied (0.8 mL) at a concentration of 100 μg/mL in buffer (PBS containing 0.05% Tween-20) containing the neuraminidase inhibitor oseltamivir carboxylate (110M). The slides were gently rocked at room temperature for 90 min followed by detection with secondary antibodies also in presence of the neuraminidase inhibitor (Table 6).

TABLE 5
Valencies of Glycan Binding Proteins

| | | | Secondary | Tertiary | |
|---|---|---|---|---|---|
| Category | GBP | Valency | Antibody | Antibody^{a} | Final |
| Plant Lectin | Con A-FITC | 4 | | | 4 |
| Plant Lectin | ECA-FITC | 2 | | | 2 |
| Human C | DC-SIGN-Fc^{b} | 2 | | | 2 |
| Type | | | | | |
| Human | CD22-Fc | 2 | α-hlgG-F^{a} | α-glgG-F^{a} | 8 |
| Siglec | | | | | |
| Human | Galectin-4- | 2 | | | 2 |
| Galectin | AF488 | | | | |
| Human IgG | Anti-CD15- | 2 | | | 2 |
| | FITC | | | | |
| Human IgG | 2G12 | 2 | α-hlgG-F^{d} | | 4 |
| Human | Serum^{c} | 2 | | | 2 |
| IgG/A/M | | | | | |
| Bacterial | Cyanovirin^{d} | 2 | | | 2 |
| GBP | | | | | |
| Viral GBP | Influenza HA | 3 | α-HA-HF^{a} | α-migG- | 12 |
| | (H3) | | | AF^{a} | |
| Intact Virus | Influenza | 500 | A-PR8 | α-rlgG-AF^{a} | 500 |
| | (PR8)^{e} | | | | |

^{a}Abbreviations used: Ab = antibody; F = FITC; AF = AF488.
^{b}After binding of DC-SIGN, binding was detected by overlay with anti-human IgG-AF488.
^{c}After binding of serum diluted 1:25 with PBS, binding was detected by overlay with goat anti-human IgG/M/A-Biotin (1:100) (Pierce) followed by Streptavidin-FITC (1:100).
^{d}After binding of CVN, binding was detected by overlay with polyclonal rabbit anti-CVN IgG-AF488 followed by anti-rabbit IgG-FITC.
^{e}After binding of virus, binding was detected by overlay with rabbit anti-PR8 followed by goat anti-rabbit IgG-AF-488.

Image acquisition and signal processing. Fluorescence intensities were detected using a ScanArray 5000 (Perkin Elmer, Boston, Mass.) confocal scanner and image analyses were carried out using ImaGene image analysis software (BioDiscovery Inc, El Segundo, Calif.). Signal to background was typically greater than 50:1 and no background subtractions were performed. Data were plotted using MS Excel software.

Results

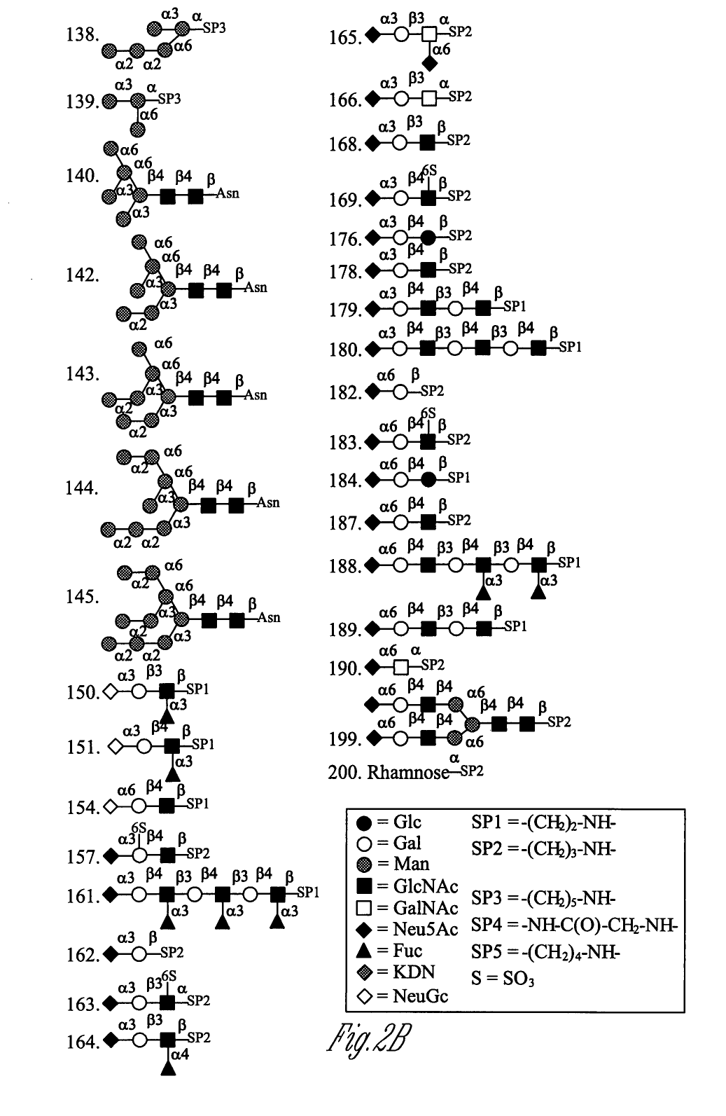

Glycan array design. The strategy adopted for covalently attaching a defined glycan library to micro-glass slides employed standard microarray printing technology as illustrated in FIG. 1. The use of an amino-reactive NHS-activated micro-glass surface allows covalent attachment of glycans containing a terminal amine by forming an amide bond under aqueous conditions at room temperature. The compound library of 200 glycoconjugates comprises diverse and biologically relevant structures representing terminal sequences of glycoprotein and glycolipid glycans. Glycan structures detected by glycan binding proteins are listed in FIG. 2 and a complete glycan listing is provided in FIG. 7. In addition, exemplary symbol structures summarizing the principal specificities of each glycan binding protein are depicted in each figure.

Optimization of glycan printing. Length of time of the printing process was a concern because the moisture sensitive NHS-slides would be exposed to air during the procedure. Binding of fluorescein-labeled concanavalin A (con A) was used as a measure of ligand coupling. Maximal binding of con A to high mannose glycans, 134-138 (structures provided in FIG. 7), was obtained at concentrations >50 μM, with less than 10% variation in maximal binding observed with printing times up to 5 hours, as shown in FIG. 13A for compound 136 (structure provided in FIG. 7). For the complete array, standard printing concentrations of 100 μM and 10 μM of each glycan were selected to represent saturating and sub-saturating levels, respectively, of the printed glycan. All samples were printed in replicates of six to generate an array of >2400 spotted ligands per glass slide, including controls.

General approach for profiling GBP specificity. In general, GBPs have low affinity for their ligands, and would not be expected to bind with sufficient avidity to withstand washing steps to remove unbound protein. For this reason, the approach routinely used was to create multi-valency as necessary to mimic the multivalent interactions that occur in nature. Some of the glycan binding proteins evaluated in these experiments and the degree of multi-valency used to achieve robust binding are summarized in Table 5. The valency required for binding ranged from 2 to 12. In several cases monovalent glycan binding proteins were evaluated as divalent recombinant Ig-Fc chimeras, and in other cases, higher valencies were achieved through the use of secondary antibodies. Binding was detected by including a fluorescent label either on the glycan binding protein or secondary antibody.

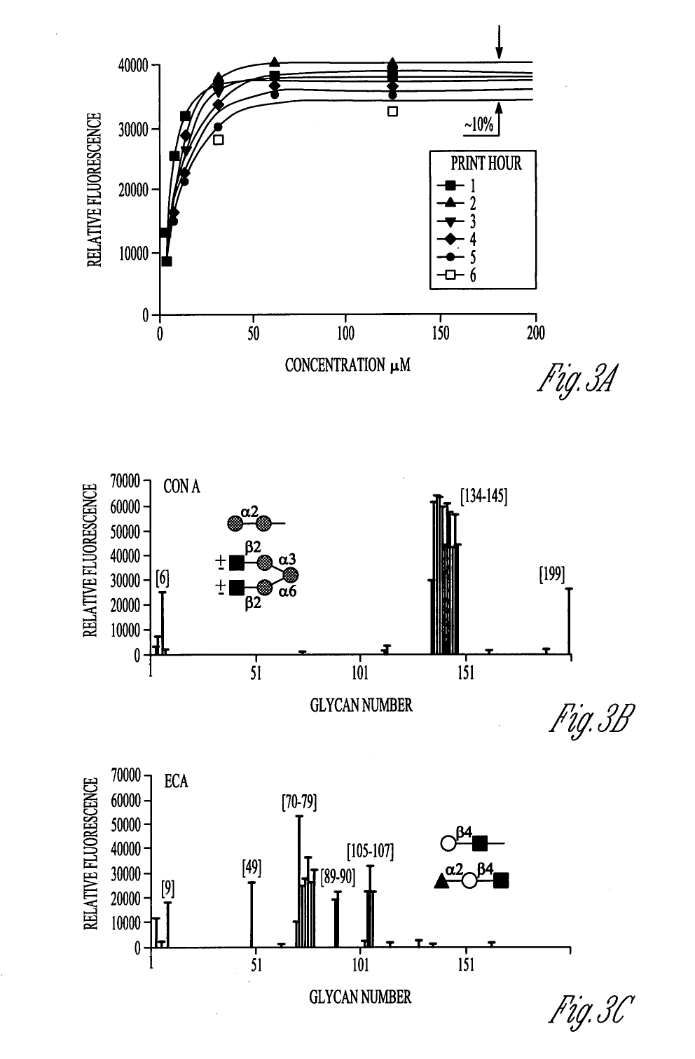

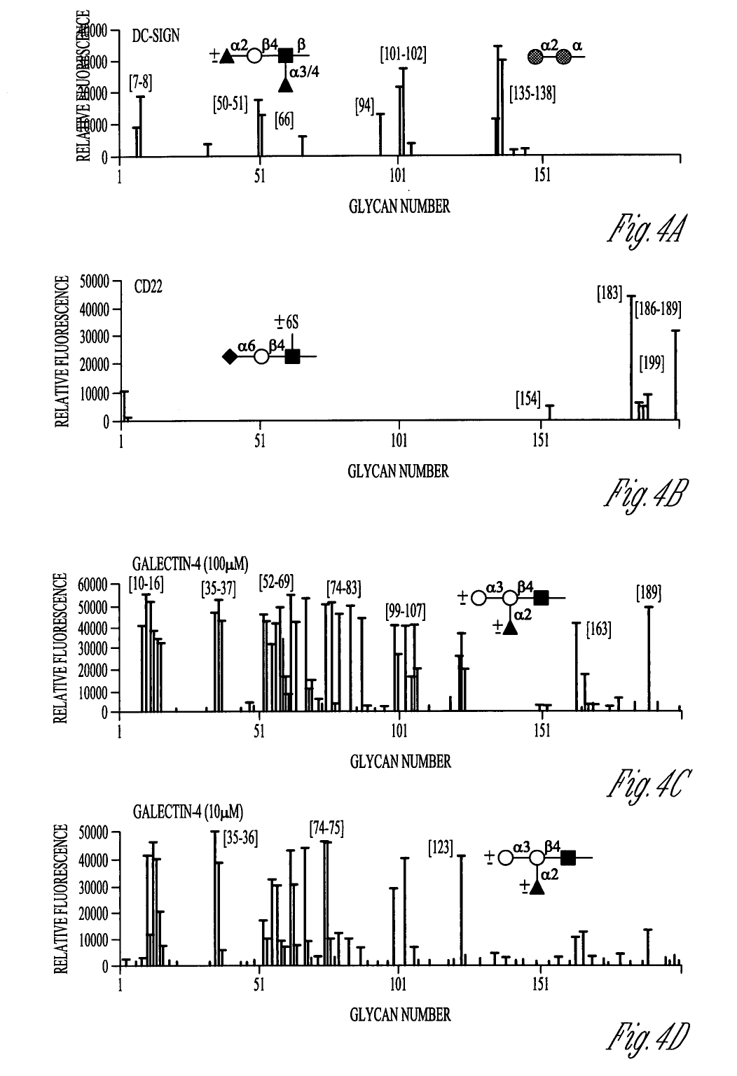

Specificity of plant lectins. As shown in FIG. 3b, two lectins, Con A and Erythrina cristagalli lectin (ECA) exhibited binding to different subsets of glycans on the array, consistent with their reported specificities. Con A bound selectively to synthetic ligands consisting of one or more α-D-mannose (Manα1-) residues as well as to isolated high-mannose N-glycans, and a bi-antennary N-linked glycan (134-145, 199, see FIG. 7). ECA bound exclusively to various terminal N-acetyllactosamine (LacNAc) structures, poly-LacNAc (9, 73, 76, see FIG. 7) and branched O-glycans (49, 72, see FIG. 7). ECA also tolerated terminal Fucα1-2Gal substitution (105-107, see FIG. 7). These specificities are consistent with those previously observed using other methodologies. See, e.g., Gupta et al. (1996) Eur. J. Biochem. 242, 320-326; Brewer et al. (1985) Biochem. Biophys. Res. Commun. 127, 1066-71; L is et al. (1987) Meth. Enzymol. 138, 544-551; Iglesias et al. (1982) Eur. J. Biochem. 123, 247-252.

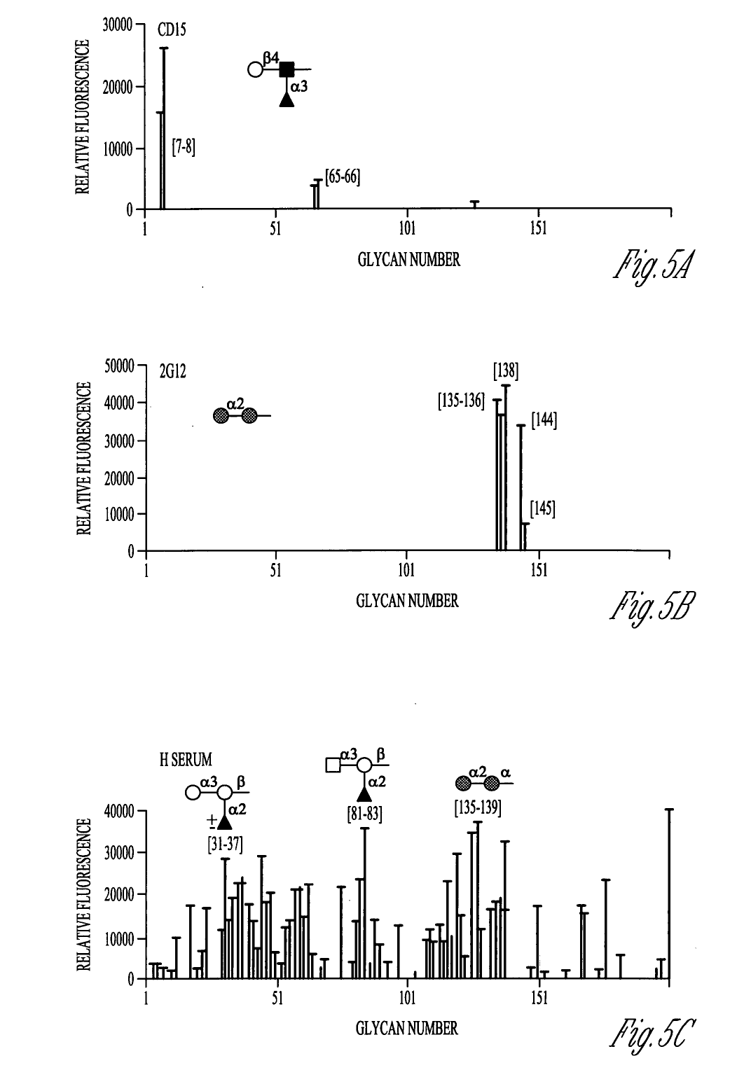

Analysis of specificities of human GBPs. Three major families of mammalian glycan binding proteins (GBPs) are involved in cell surface biology through recognition of glycan ligands—C-type lectins, siglecs and galectins. One exemplary member from each class was selected for analysis (FIG. 4).

DC-SIGN, a member of the group 2 subfamily of the C-type lectin family, is a dendritic cell protein implicated in innate immunity and the pathogenicity of human immunodeficiency virus-1 (HIV-1) (Kooyk, Y. & Geijtenbeek, T. B. (2002) Immunol. Rev. 186, 47-56). As shown in FIG. 4, a recombinant DC-SIGN-Fc recognized two classes of glycans, various fucosylated oligosaccharides with the Fucα1-3GlcNAc and Fucα1-4GlcNAc oligosaccharides found as terminal sequences on N- and O-linked oligosaccharides (7, 8, 51, 66, 94, 102, see FIG. 7), and mannose containing oligosaccharides terminated with Manα1-2-residues (135-138, 144, 145, see FIG. 7), consistent with specificities found by other groups, for example, as described in Guo et al. (2004) Nat. Struct. Mol. Biol. 11, 591-8; van Die et al. (2003) Glycobiology 13, 471-478; and Adams et al. (2004) Chem.l Biol. 11, 875-81

CD22, a member of the immunoglobulin superfamily lectins (Siglecs), is a well-known negative regulator of B cell signaling and binds selectively to glycans with Siaα2-6Gal-sequences. Blixt et al. (2003) J. Biol. Chem. 278, 31007-19; Engel et al. (1993) J. Immunol. 150, 4719-4732; Kelm et al. (1994) Curr. Biol. 4, 965-72; Powell et al. (1993) J. Biol. Chem. 268, 7019-7027. CD22 bound exclusively to the seven structures containing the terminal Siaα2-6Galβ1-4GlcNAc-sequence including a bi-antennary N-linked glycan (154, 187-189 and 199, see FIG. 7). An additional 6-O-GlcNAc-sulfation (Neu5Acα2-6Galβ1-4[6Su]GlcNAc-183, see FIG. 7) appeared to enhance binding relative to the corresponding non-sulfated glycan, suggesting that this glycan could be a preferred ligand for human CD22.

Galectins are a family of β-galactoside binding lectins that bind terminal and internal galactose residues. See, Hirabayashi et al. (2002) Biochim. Biophys. Acta 1572, 232-54. Galectin-4 has been identified as a possible intracellular mediator with anti-apoptotic activity. Huflejt et al. (1997) J. Biol. Chem. 272, 14294-303; Huflejt, M. E. & Leffler, H. (2004) Glycoconjugate J. 20, 247-55.

By comparing Galectin-4 binding to saturated glycans (printed at 100 μM concentration) with binding to sub-saturated glycans (printed at 10 μM concentration), preferred binding specificities were revealed. In particular, Galα1-3-linked to lactose (35-37), Fucα1-2-linked to lac(NAc) (100, 103, 105-107), or R-GlcNAcβ1-3-linked to lactose (123), as well as 3′-sulfation (11-16) substantially enhanced the affinity. This specificity profile is similar to that reported for a rat ortholog of Galectin-4. See Wu et al. (2004) Biochimie 86, 317-26; Oda et al. (1993) J. of Biol. Chem. 268, 5929-5939.

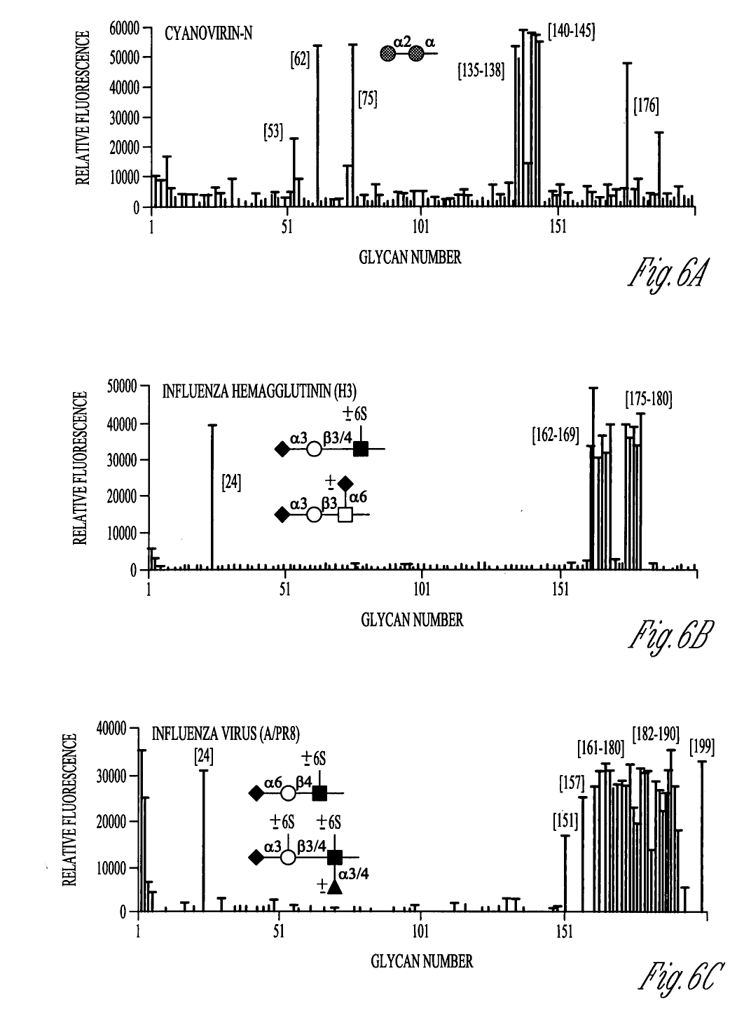

Glycan specific antibodies. monoclonal and polyclonal anti-glycan antibodies from three different sources were also analyzed (FIG. 5). The commercial leukocyte differentiation antigen CD-15 has been documented to recognize a carbohydrate antigen, Lewis^{x} (Galβ1-4[Fucα1-3]GlcNAc). When evaluated on the array described herein this antibody was highly specific for Lewis^{x} structures (7, 8, 66, see FIG. 7), and did not recognize the same structure modified by additional sialylation (161), sulfation (26), fucosylation (102) or LacNAc extension (73) (see FIG. 7 for structures).

One of the most studied human anti-HIV monoclonal antibodies is 2G12, which neutralizes a broad spectrum of natural HIV isolates via recognition of high mannose type N-linked glycans on the major envelope glycoprotein, gp120. Lee et al. (2004) Angew. Chem. Int. Ed. 43, 1000-1003; Calarese et al. (2003) Science 300, 2065-71; Scanlan et al., (2002) J. Virol. 76, 7306-21; Sanders (2002) J. Virol. 76, 7293-305; Trkola et al. (1996) J. Virol. 70, 1100-8. The glycan array contains a variety of synthetic mannose fragments with the natural series of high mannose N-glycans (Man5-Man9) isolated from ribonuclease B. Recombinant 2G12 exhibited strong binding of synthetic Manα1-2-terminal mannose oligosaccharides (135, 136, 138). See also Bryan et al. (2004) J. Am. Chem. Soc. 126, 8640-41; Lee et al. (2004) Angew. Chem. Int. Ed. 43, 1000-1003; Adams et al. (2004) Chem.l Biol. 11, 875-81. In addition, of the series of natural high mannose type N-glycans, 2G12 exhibited preferred binding to Man8 glycans (144) relative to Man5, Man6, Man7 or Man9 glycans (140, 142, 143, 145) (see FIG. 7 for these structures).

In particular, the glycans to which the 2G12 antibodies bound had any the following Man-8 N-glycan structures, or were a combination thereof:

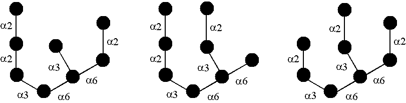

wherein each filled circle (●) represents a mannose residue.

A smaller level of binding was observed between the 2G12 antibodies and Man-9-N-glycans. As shown in Table 6, simpler synthetic glycans bind 2G12 as well as the Man8 glycans. However, the simpler compounds are more likely to elicit an immune response that will generate antibodies to the immunogen, but not the high mannose glycans of the gp120. The natural structure is also less likely to produce an unwanted immune response. Indeed, yeast mannan is a polymer of mannose and is a potent immunogen in humans, representing a major barrier to production of recombinant therapeutic glycoproteins in yeast.

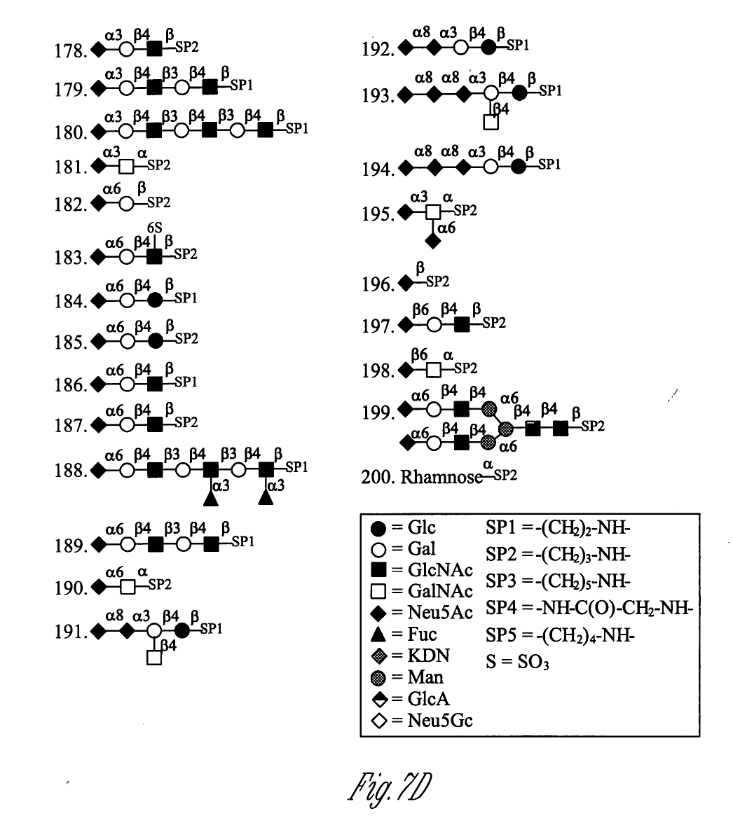

TABLE 6
Summary of the binding of 2G12 to mannose containing glycans in the
glycan array shown in FIG. 8. Samples 1-6 are glycoproteins, samples
134-139 are synthetic high mannose glycans, samples 140-145 are
natural high mannose glycopeptides isolated from bovine
ribonuclease, and sample 199 is a bi-antennary complex type
glycan terminated in sialic acid. Relative binding activity:
− = <1000; + = 1000-6000; ++ 6000-25,000; and +++ >25,000.

| No. | Mannose containing ligands | Rel. spec. |
|---|---|---|
| 1 | Alpha1-acid glycoprotein | − |
| 2 | Alpha1-acid glycoprotein A | − |
| 3 | Alpha1-acid glycoprotein B | − |
| 4 | Ceruloplasmin | − |
| 5 | Transferrin | − |
| 6 | Fibrinogen | − |
| 134 | Ma#sp3 | − |
| 135 | Ma2Ma2Ma3Ma#sp3 | +++ |
| 136 | Ma2Ma3[Ma2Ma6]Ma#sp3 | +++ |
| 137 | Ma2Ma3Ma#sp3 | − |
| 138 | Ma3[Ma2Ma2Ma6]Ma#sp3 | +++ |
| 139 | Ma3[Ma6]Ma#sp3 | − |
| 140 | Man-5#aa | − |
| 142 | Man-6#aa | − |
| 143 | Man-7#aa | − |
| 144 | Man-8#aa | +++ |
| 145 | Man-9#aa | + |
| 199 | OS-11 | − |

These results indicate that glycans with eight mannose residues are superior antigens for binding the 2G12 anti-HIV neutralizing antibodies.

To test the array against more complex samples, anti-glycan antibodies present in human serum were investigated. Following incubation with serum, bound IgG, IgA and IgM were detected using labeled anti-human IgG/A/M antibody. A surprising diversity of antibody specificities was observed. It was remarkably consistent among samples from ten individuals as indicated in FIG. 5. This profile of human anti-glycan antibodies detects the ABO blood group fragments (variously represented in different individuals) (32, 81, 83), mannose fragments (135-139), α-Gal-(31-37) and ganglioside-epitopes (55-59, 132, 168), as well as fragments of the gram negative bacterial cell wall peptidoglycan (127) and rhamnose (200) (see FIG. 7 for these structures). Notably, glycans containing the Galβ1-3GlcNAc sub-structure were consistently detected (12, 61, 62, 132, 150, 168) except when fucosylated (25, 51, 94, 100) thus generating the human blood group antigens H, Lewis^{a} or Lewis^{b} (see FIG. 7 for structures). All of these structures can be identified as either blood group antigens or fragments of microorganisms (e.g. bacteria, yeast etc.) to which humans are exposed.

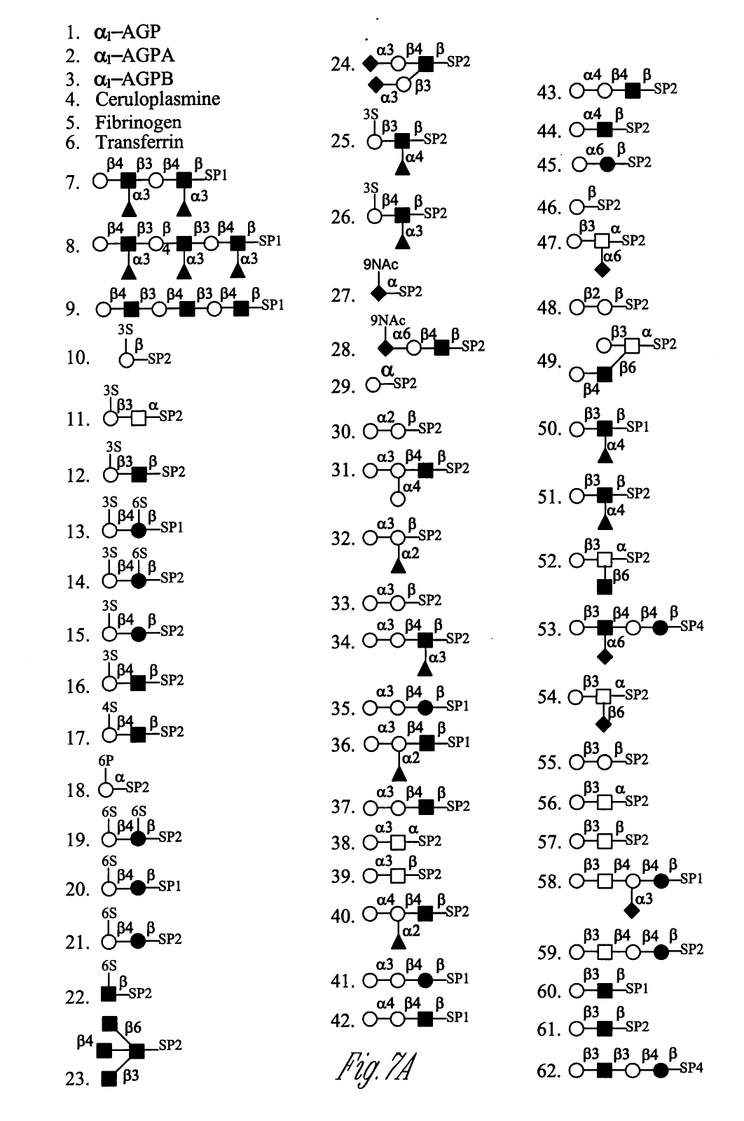

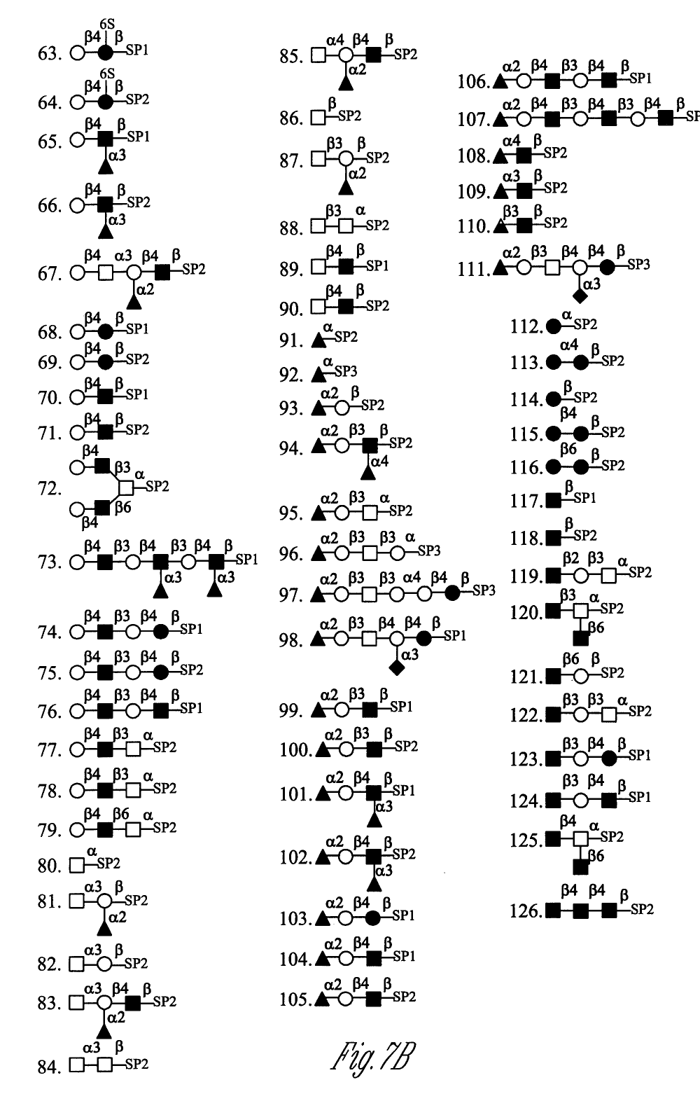

Analysis of bacterial and viral GBPs. Cyanovirin-N(CVN) is a cyanobacterial protein that can block the initial step of HIV-1 infection by binding to high mannose groups on the envelope glycoprotein gp120. Adams et al. (2004) Chem. Biol 11, 875-81; Bewely, C. A. & Otero-Quintero, S. (2001) J. Am. Chem. Soc. 123, 3892-3902. On the array, CVN specifically recognized the synthetic fragments bearing terminal Manα1-2-residues (135-138), as well as high mannose glycans with one or more Manα1-2-termini (140-145), in keeping with its reported specificity (FIGS. 6 and 7). In addition, CVN bound to several lacto- and neolacto-structures (53, 62, 75, 176, see FIGS. 6 and 7).

Influenza viruses exhibit specificity in their ability to recognize sialosides as cell surface receptor determinants through the viral binding protein, the hemagglutinin. Depending on the species of origin, the hemagglutinin has specificity for sialosides with sialic acid in the NeuAcα2-3Gal or NeuAcα2-6Gal linkage. Connor et al. (1994) Virol 205, 17-23; Rogers, G. N. & D'Souza, B. L. (1989) Virol. 173, 317-22; Rogers et al. (1983) Nature 304, 76-8. While the intrinsic affinity of sialosides for the hemagglutinin is weak (Kd≈2 mM), binding is strengthened through polyvalent interactions at the cell surface. Sauter et al. (1989) Biochem. 28, 8388-96.

Results shown in FIG. 6 reveal the binding of a recombinant avian H3 hemagglutinin (Duck/Ukraine/1/63) bound to Neu5Acα2-3-linked to galactosides (24, 162-169, 176-180, see FIG. 7), but not to anyNeu5Acα2-6- or Neu5Acα2-8-linked sialosides. Intact influenza viruses, such as A/Puerto Rico/8/34 (H1N1), were also strongly bound to the array. The overall affinities are consistent with previous findings and show specificity for both a2-3 and a2-6 sialosides. Rogers, G. N. & Paulson, J. C. (1983) Virol. 127, 361-73.

Detailed fine specificities were also revealed such as binding to Neu5Acα2-3- and Neu5Acα2-6-linked to galactosides (24, 151, 157, 161-180, 182-190, 199, see FIG. 7), as well as certain O-linked sialosides.

Thus, the glycan microarrays described herein utilize standard robotic printing, scanning and image analysis software used for DNA microarrays. The combination of using amine-functionalized glycans with the NHS-activated glass surface results in robust and reproducible covalent attachment of glycans with no modifications of standard DNA printing protocols. The array can be used with no further preparation of the surface for assessing the specificity of a wide variety of glycan binding proteins, yielding uniformly low backgrounds regardless of the labeled protein used for detection. Moreover, only 0.1-2 μg of glycan binding protein is needed for optimal signal, over 100-fold less than required for an ELISA based array that uses predominately the same glycan library. Fazio et al. (2002) J. Am. Chem. Soc. 124, 14397-14402. The arrays performed well for a wide variety of glycan binding proteins, confirming primary specificities documented by other means, and revealing novel aspects of fine specificity that had not previously been recognized.

### EXAMPLE 6

### Diagnosis of Neoplasia Using Glycan Arrays

This Example illustrates that antibodies present in breast cancer patients can be detected using the glycan arrays of the invention. Only a small sample volume of human serum (e.g., about 10 il to 50 il) was needed for detecting antibodies that bound to specific types of glycans. Thus, the invention provides non-invasive screening procedures for detecting breast neoplasia.

Materials and Methods:

Individual (not pooled) sera were collected from 9 patients who were diagnosed with metastatic breast cancer (MBC). Blood samples were collected before treatment, so that therapeutic intervention would not interfere with patient immune responses. One patient with breast cancer but with good prognosis (IDC, Stage 1) was also included in the study. As control, or “healthy” sera, sera from ten healthy individuals, 5 female and 5 male, with no known malignancies was collected.

Sera were diluted 1:25 with PBS containing 3% BSA, and placed on the glycan array slide in humidified chamber at room temperature for 90 min. The glycan array slide was then rinsed gently with PBS/0.05% Tween, incubated with biotinylated goat antibody against human IgG, IgM and IgA, rinsed in PBS/0.05% Tween, and incubated with streptavidin-Alexa488 fluorescent dye. Following rinses in PBS/0.05% Tween and H₂O, glycan array slides were dried and scanned using the commercial DNA array scanner. The images were analyzed and intensity of fluorescence in spots corresponding to the antibodies bound to the individual glycans was quantified using a ScanArray 5000 (Perkin Elmer, Boston, Mass.) confocal scanner and image analyses were carried out using ImaGene image analysis software (BioDiscovery Inc, El Segundo, Calif.). Signal to background was typically greater 50:1 and no background subtractions were performed. Data were plotted using MS Excel software.

Results

The results of these experiments are provided in FIGS. 8-10. A profile of the relative fluorescence intensity of labeled antibodies bound to specific glycans on the array is provided in FIG. 8. As illustrated in FIG. 8, there are significant differences between the reactivity of sera from controls and from patients with metastatic breast cancer. In particular, the levels of certain anti-carbohydrate antibodies are much higher in patients with metastatic breast cancer. Glycans to which antibodies from metastatic breast cancer patients bind include ceruloplasmin, Neu5Gc(2-6)GalNAc, GM1, Sulfo-T, Globo-H, sialylated Tn (Neu5Ac-alpha6-GalNAc-alpha) and LNT-2. In addition, the following glycans will also bind to antibodies obtained from breast cancer patients: Tri-LacNAc (glycan 9 of Table 1), LacNAc-LeX-LeX (glycan 73), LacNAc-LacNAc (glycan 76), H-type-2-LacNAc (glycan 106), H-type2-LacNAc-LacNAc (glycan 107), GlcNAcβ3LacNAc (glycan 124), SLeXLacNAc (glycan 174), 3′SialylDiLacNAc (glycan 179), 3′Sialyl-tri-LacNAc (glycan 180), 6Sia-LacNAc-LeX-LeX (glycan 188), and/or 6SiaLacNAc-LacNAc (glycan 189). Each glycan number indicates a corresponding glycan listed in Table 1. Structures for these glycans are shown in FIG. 11.

GM1 has the following structure: Gal-beta3-GalNAc-beta-4-[Neu5Ac-alpha3]-Gal-beta-4-Glc-beta.

The sulfo-T antigens are T-antigens with sulfate residues. In general, T antigens have the structure Galβ3GalNAc and can have various modifications. LNT-2 is a ligand for tumor-promoting Galectin-4. See Huflejt & Leffler (2004) Glycoconjugate J, 20: 247-255).

The structure of LNT-2 includes the following glycan: GlcNAc-beta3-Gal-beta-4-Glc-beta.

Globo-H has the following structure: Fucose-alpha2-Gal-beta3-GalNAc-beta3-Gal-alpha-4-Gal-beta-4-Glc.

The antibodies that bind to these glycans therefore react with a series of glycan types. The clusters of glycans reactive with these antibodies define the neoplasia status more precisely then would detection of an individual antibody alone. Moreover, the levels of the antibodies reactive with individual glycan clusters can be quantified and converted into score values used for mathematical and statistical serum sample analysis that would allow diagnostic assignment of the neoplasia risk for the individual patient, when compared with the value range characteristic of the individuals with no known neoplasia.

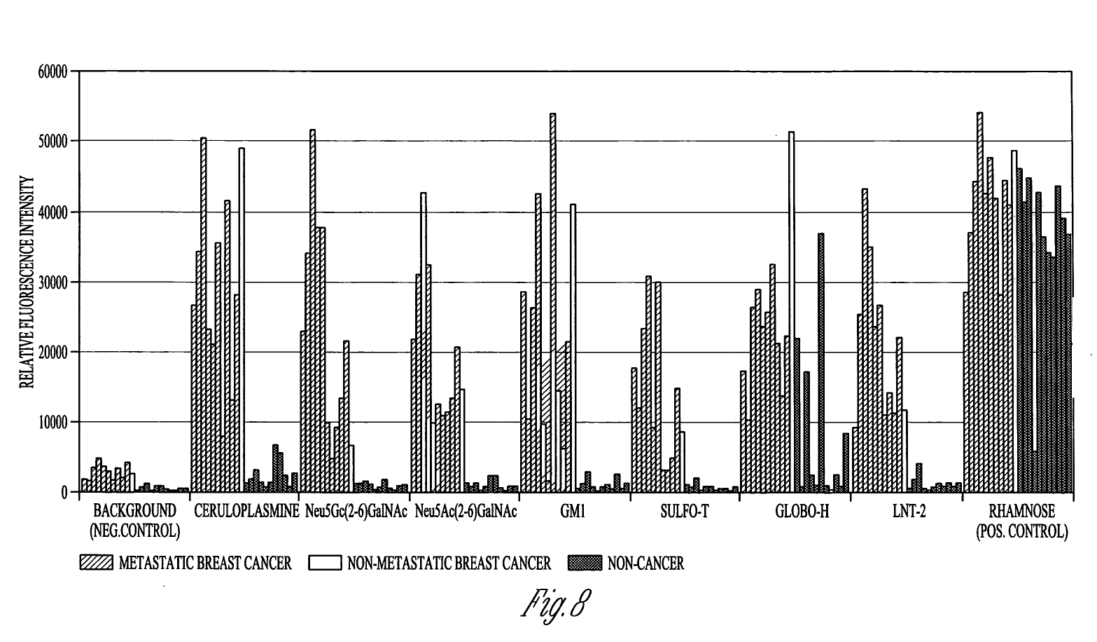

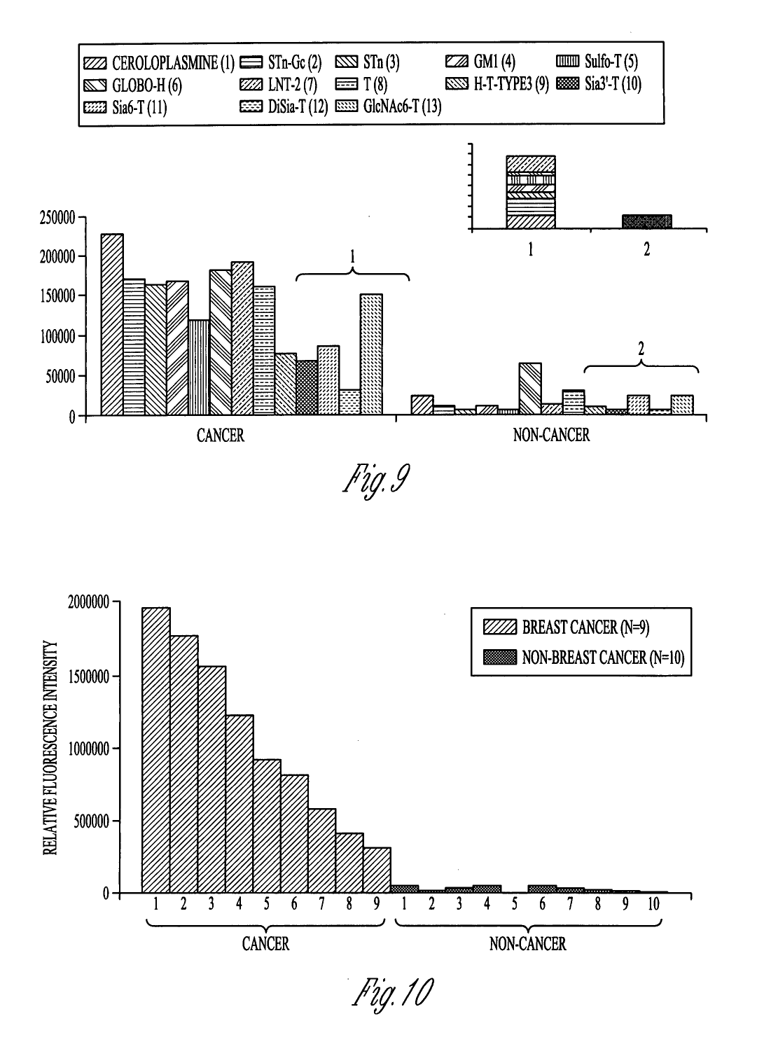

Specifically, antibodies against ceruloplasmin (FIG. 8, compound no. 2) and against cancer specific carbohydrate antigen Neu5Acα2-6GalNAcα-(STn-, FIG. 8, compound no. 3 and 4) appear at significantly higher levels in all MBC patients as compared to “healthy” individuals. There are also antibodies against other specific glycans that are present in metastatic breast cancer patients at the levels higher than in the healthy individuals. These specific glycan categories include: a group of T-antigens carrying various modifications (see FIG. 9, compounds no. 5, 8-13), LNT-2 (a known ligand for tumor-promoting Galectin-4, Huflejt and Leffler, 2004), Globo-H-, and GM1-antigens.

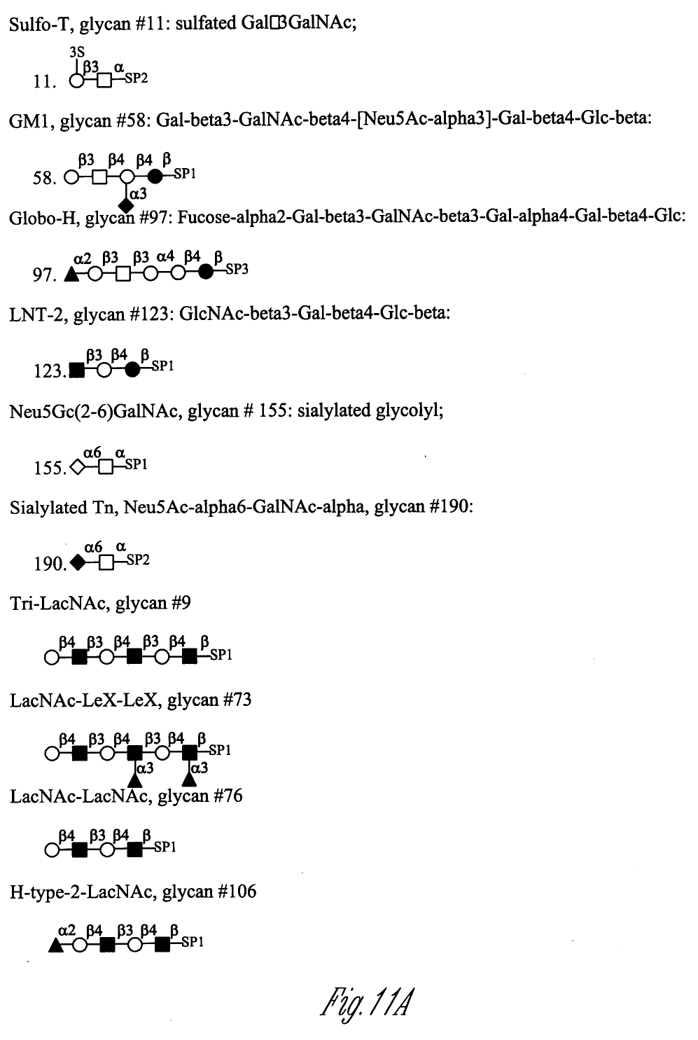

As shown in FIG. 10, combining the relative fluorescence intensities corresponding to the levels of serum antibodies listed in FIG. 9 for each patient allows generation of the antibody signal range that provides a clear distinction between breast cancer and non-cancer population. There fore, this test can provide an additional tool for appropriate correlation between specific glycoprotein profiles and various stages of disease to allow for identification of appropriate therapeutic targets.

These findings suggest that more than one glycan is present as a naturally occurring epitope during malignant transformation in breast cancer patients and these epitopes elicit immune response in each of the so far examined (breast) cancer patients. Moreover, these results indicate that clusters of different antibodies reactive against tumor-associated glycans can be detected simultaneously in the individual patient sera. Such detection of several antibody types provides much better diagnostic information than information about the presence of a single type of antibody reactive with a single type of glycan.

These combined tumor-associated glycans will be the preferred immunogen for a vaccine composition to elicit an immune response that results in production of antibodies neutralizing antibodies activities of tumor-promoting glycans. Such compositions will likely include multivalent glycans to mimic the clustered N-linked glycan epitopes on cellular surfaces of cancer, stromal, and endothelial cells.

### REFERENCES

1. Hakomori, S.-I. (2001) in The Molecular Immunology of Complex Carbohydrates-2, ed. Wu, A. M. (Kluwer Academic/Plenum, publishers, pp. 369-402.
2. Taylor, M. E. & Drickamer, K. (2003) Introduction to Glycobiology (Oxford University Press, Oxford).
3. Mrksich, M. (2004) Chem. Biol. 11, 739-40.
4. Feizi, T., Fazio, F., Wengang, C. & Wong, C.-H. (2003) Curr. Opin. Struct. Biol. 13, 637-645.
5. Drickamer, K. & Taylor, M. E. (2002) Genomebiology 3, 1034.1-4.
6. Love, K. R. & Seeberger, P. H. (2002) Angew. Chem. Int. Ed. 41, 3583-3586.
7. Galanina, O. E., Mecklenburg, M., Nifantiev, N. E., Pazynina, G. V. & Bovin, N. V. (2004) Lab Chip 3, 260-5.
8. Guo, Y., Feinberg, H., Conroy, E., Mitchell, D. A., Alvarez, R., Blixt, O., Taylor, M. E., Weis, W. I. & Drickamer, K. (2004) Nat. Struct. Mol. Biol. 11, 591-8.
9. Wang, D., Liu, S., Trummer, B. J., Deng, C. & Wang, A. (2002) Nat. Biotech. 20, 275-281.
10. Fukui, S., Feizi, T., Galustian, C., Lawson, A. M. & Chai, W. (2002) Nat. Biotech. 20, 1011-1017.
11. Willats, W. G. T., Rasmussen, S. E., Kristensen, T., Mikkelsen, J. D. & Knox, J. P. (2002) Proteomics 2, 1666-1671.
12. Fazio, F., Bryan, M. C., Blixt, O., Paulson, J. C. & Wong, C.-H. (2002) J. Am. Chem. Soc. 124, 14397-14402.
13. Nimrichter, L., Gargir, A., Gortler, M., Alstock, R. T., Shtevi, A., Weisshaus, O., Fire, E., Dotan, N. & Schnaar, R. L. (2004) Glycobiology 14, 197-203.
14. Bryan, M. C., Fazio, F., Lee, H. K., Huang, C. Y., Chang, A. Y., Best, M. D., Calarese, D. A., Blixt, O., Paulson, J. C., Burton, D. R., Wilson, I. A. & Wong, C.-H. (2004) J. Am. Chem. Soc. 126, 8640-41.
15. Park, S., Lee, M. R., Pyo, S. J. & Shin, I. (2004) J. Am. Chem. Soc. 126, 4812-9.
16. Ratner, D. M., Adams, E. W., Su, J., O'Keefe, B. R., Mrksich, M. & Seeberger, P. H. (2004) Chembiochem 5, 379-82.
17. Schwarz, M., Spector, L., Gargir, A., Shtevi, A., Gortler, M., Alstock, R. T., Dukler, A. A. & Dotan, N. (2003) Glycobiology 13, 749-754.
18. Houseman, B. T. & Mrksich, M. (2002) Chem. Biol. 9, 443-454.
19. Park, S. & Shin, I. (2002) Angew. Chem. Int. Ed. 41, 3180-3182.
20. Bergh, A., Magnusson, B. G., Ohlsson, J., Wellmar, U. & Nilsson, U. J. (2001) Glycoconj. J. 18, 615-21.
21. Shiyan, S. D. & Bovin, N. V. (1997) Glycoconj. J. 14, 631-8.
22. Pazynina, G. V., Sablina, M. A., Tuzikov, A. B., Chinarev, A. A. & Bovin, N. V. (2003) Mendeleev Commun. 13, 245-248.
23. Pazynina, G. V., Tyrtysh, T. V. & Bovin, N. V. (2002) Mendeleev Commun. 12, 183-184.
24. Pazynina, G. V., Tuzikov, A. B., Chinarev, A. A., Obukhova, P. & Bovin, N. V. (2002) Tet. Lett. 43, 8011-8013.
25. Nifant'ev, N. E., Tsvetkov, Y. E., Shashkov, A. S., Kononov, L. O., Menshov, V. M., Tuzikov, A. B. & Bovin, N. V. (1996) J. Carbohydr. Chem. 15, 939-953.
26. Zemlyanukhina, T. V., Nifant'ev, N. E., Shashkov, A. S., Tsvetkov, Y. E. & Bovin, N. V. (1995) Carbohydr. Lett. 1, 277-284.
27. Lee, H. K., Scanlan, C. N., Huang, C. Y., Chang, A. Y., Calarese, D. A., Dwek, R. A., Rudd, P. M., Burton, D. R., Wilson, I. A. & Wong, C. H. (2004) Angew. Chem. Int. Ed. 43, 1000-1003.
28. van Die, I., van Vliet, S. J., Nyame, A. K., Cummings, R. D., Bank, C. M. C., Appelmelk, B. J., Geijtenbeek, T. B. & Kooyk, Y. (2003) Glycobiology 13, 471-478.
29. Gamblin, S. J., Haire, L. F., Russell, R. J., Stevens, D. J., Xiao, B., Ha, Y., Vasisht, N., Steinhauer, D. A., Daniels, R. S., Elliot, A., Wiley, D. C. & Skehel, J. J. (2004) Science 303, 1838-42.
30. Calarese, D. A., Scanlan, C. N., Zwick, M. B., Deechongkit, S., Mimura, Y., Kunert, R., Zhu, P., Wormald, M. R., Stanfield, R. L., Roux, K. H., Kelly, J. W., Rudd, P. M., Dwek, R. A., Katinger, H., Burton, D. R. & Wilson, I. A. (2003) Science 300, 2065-71.
31. Scanlan, C. N., Pantophlet, R., Wormald, M. R., Ollmann Saphire, E., Stanfield, R., Wilson, I. A., Katinger, H., Dwek, R. A., Rudd, P. M. & Burton, D. R. (2002) J. Virol. 76, 7306-21.
32. Blixt, O., Collins, B. E., van den Nieuwenhof, I. M., Crocker, P. R. & Paulson, J. C. (2003) J. Biol. Chem. 278, 31007-19.
33. Huflejt, M. E., Jordan, E. T., Gitt, M. A., Barondes, S. H. & Leffler, H. (1997) J. Biol. Chem. 272, 14294-303.
34. Ting, C. A., Lee, S.-F. & Wang, K. (2003) BioTechniques 35, 808-810.
35. Blixt, O. & Razi, N. (2004) in Synthesis of Carbohydrates through Biotechnology, eds. Wang, P. G. & Ichikawa, Y. (American Chemical Society, Washington D.C.), Vol. 873, pp. 93-112.
36. Collins, B. E. & Paulson, J. C. (2004) Curr. Opin. Chem. Biol. in press.
37. Gupta, D., Oscarson, S., Raju, T. S., Stanely, P., Toone, E. J. & Brewer, C. F. (1996) Eur. J. Biochem. 242, 320-326.
38. Brewer, F., Bhattacharyya, L., Brown, R. D. & Koenig, S. H. (1985) Biochem. Biophys. Res. Commun. 127, 1066-71.
39. L is, H. & Sharon, N. (1987) Meth. Enzymol. 138, 544-551.
40. Iglesias, J. L., L is, H. & Sharon, N. (1982) Eur. J. Biochem. 123, 247-252.
41. Kooyk, Y. & Geijtenbeek, T. B. (2002) Immunol. Rev. 186, 47-56.
42. Adams, E. W., Ratner, D. M., Bokesch, H. R., McMahon, J. B., O'Keefe, B. R. & Seeberger, P. H. (2004) Chem.l Biol. 11, 875-81.
43. Engel, P., Nojima, Y., Rothstein, D., Zhou, L. J., Wilson, G. L., Kehrl, J. H. & Tedder, T. F. (1993) J. Immunol. 150, 4719-4732.
44. Kelm, S., Pelz, A., Schauer, R., Filbin, M. T., Tang, S., de Bellard, M. E., Schnaar, R. L., Mahoney, J. A., Hartnell, A., Bradfield, P. & Crocker, P. R. (1994) Curr. Biol. 4, 965-72.
45. Powell, L. D., Sgroi, D., Sjoberg, E. R., Stamenkovic, I. & Varki, A. (1993) J. Biol. Chem. 268, 7019-7027.
46. Hirabayashi, J., Hashidate, T., Arata, Y., Nishi, N., Nakamura, T., Hirashima, M., Urashima, T., Oka, T., Futai, M., Muller, W. E., Yagi, F. & Kasai, K. (2002) Biochim. Biophys. Acta 1572, 232-54.
47. Huflejt, M. E. & Leffler, H. (2004) Glycoconjugate J. 20, 247-55.
48. Wu, A. M., Wu, J. H., Liu, J. H., Singh, T., Andre, S., Kaltner, H. & Gabius, H. J. (2004) Biochimie 86, 317-26.
49. Oda, Y., Herrmann, J., Gitt, M. A., Turck, C. W., Burlingame, A. L., Barondes, S. H. & Leffler, H. (1993) J. of Biol. Chem. 268, 5929-5939.
50. Sanders, R. W., Venturi, M., Schiffner, L., Kalyanaraman, R., Katinger, H., Lloyd, K. O., Kwong, P. D. & Moore, J. P. (2002) J. Virol. 76, 7293-305.
51. Trkola, A., Purtscher, M., Muster, T., Ballaun, C., Buchacher, A., Sullivan, N., Srinivasan, K., Sodroski, J., Moore, J. P. & Katinger, H. (1996) J. Virol. 70, 1100-8.
52. Bewely, C. A. & Otero-Quintero, S. (2001) J. Am. Chem. Soc. 123, 3892-3902.
53. Connor, R. J., Kawaoka, Y., Webster, R. G. & Paulson, J. C. (1994) Virol 205, 17-23.
54. Rogers, G. N. & D'Souza, B. L. (1989) Virol. 173, 317-22.
55. Rogers, G. N., Paulson, J. C., Daniels, R. S., Skehel, J. J., Wilson, I. A. & Wiley, D. C. (1983) Nature 304, 76-8.
56. Sauter, N. K., Bednarski, M. D., Wurzburg, B. A., Hanson, J. E., Whitesides, G. M., Skehel, J. J. & Wiley, D. C. (1989) Biochem. 28, 8388-96.
57. Rogers, G. N. & Paulson, J. C. (1983) Virol. 127, 361-73.
58. Vallee, F., Karaveg, K., Herscovics, A., Moremen, K. W., and Howell, P. L. (2000). Structural basis for catalysis and inhibition of N-glycan processing class 1 alpha 1,2-mannosidases. J Biol Chem 275, 41287-41298.
59. Tremblay, L. O., and Herscovics, A. (2000). Characterization of a cDNA encoding a novel human Golgi alpha 1,2-mannosidase (IC) involved in N-glycan biosynthesis. J Biol Chem 275, 31655-31660.
60. Blixt, O., Head, S, Mondala T, Scanlan C N, Huflejt M E, Bovin N, Calarese D, Alvarez R, Stevens J, Bryan M C, Fazio F, Razi N, Cummings R, van Die I, Burton D, Wilson I C, Wong C.-H, and Paulson J C. Printed Covalent Glycan Array for Ligand Profiling of Diverse Carbohydrate Binding Proteins. Manuscript submitted to PNAS.
61. Hakomori S. 2001. Tumor-associated carbohydrate antigens defining tumor malignancy: basis for development of anti-cancer vaccines. Adv Exp Med Biol. 491: 369-402.
62. Hakomori S. 1996. Tumor malignancy defined by aberrant glycosylation and sphingo(glyco)lipid metabolism. Cancer Res. 56: 5309-18.
63. Senra Varela, A., Bosco Lopez Saez J J, and Quintela Sera D. 1997. Serum ceruloplasmin as a diagnostic marker of cancer. Cancer Lett. 121: 139-145.
64. Koenig A, Jain R, Vig R, Norgard-Sumnicht K E, Matta K L, Varki A. 1997. Selectin inhibition: synthesis and evaluation of novel sialylated, sulfated and fucosylated oligosaccharides, including the major capping group of GlyCAM-1. Glycobiology. 7:79-93.
65. Sarkar A K, Rostand K S, Jain R K, Matta K L, Esko J D. 1997. Fucosylation of disaccharide precursors of sialyl Lewis X inhibit selectin-mediated cell adhesion. J Biol. Chem. 272: 25608-16.
66. Huflejt, M E., and Leffler, H. Galectin-4 in normal tissues and cancer. (2004). Glycoconjugate J. 20: 247-255.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. An array of glycan molecules for detecting breast cancer in a patient test sample comprising a solid support and a library of glycan molecules, wherein at least one of the glycan molecules bind antibodies previously identified as being associated with neoplasia in patients with benign, pre-malignant or malignant breast tumors.

2. The array of claim 1, wherein the glycans are selected from the group consisting of ceruloplasmin, Neu5Gc(2-6)GalNAc, GM1, Sulfo-T, Globo-H, sialylated Tn (Neu5Ac-alpha6-GalNAc-alpha) and LNT-2.

3. The array of claim 1, wherein the glycans are selected from the group consisting of Tri-LacNAc, LacNAc-LeX-LeX, LacNAc-LacNAc, H-type-2-LacNAc, H-type2-LacNAc-LacNAc, GlcNAcβ3LacNAc, SLeXLacNAc, 3′SialylDiLacNAc, 3′Sialyl-tri-LacNAc, 6Sia-LacNAc-LeX-LeX and 6SiaLacNAc-LacNAc.

4. The array of claim 1, wherein each type of glycan in the library is attached to a solid support at a defined glycan probe location, wherein each glycan probe location defines a region of the solid support that has multiple copies of one type of similar glycan molecules attached thereto.

5. The array of claim 1, wherein the array is a microarray.

6. The array of claim 1, wherein the test sample is a bodily fluid.

7. The array of claim 1 wherein the test sample is a blood sample, a serum sample, a plasma sample, a urine sample, a breast milk sample, a breast secretion sample, a nipple aspirate sample, an ascites fluid sample, a plural ascites fluid sample, a saliva sample, a cerebrospinal fluid sample, a vaginal secretion sample, an ovarian fluid sample or a tissue sample.

8. The array of claim 1, wherein the GM1 is Gal-beta3-GalNAc-beta-4-[Neu5Ac-alpha3]-Gal-beta-4-Glc-beta; wherein the Sulfo-T comprises Galβ3GalNAc, where sulfate may be a substituent on one or more galactose residues; wherein the Globo-H comprises Fucose-alpha2-Gal-beta3-GalNAc-beta3-Gal-alpha-4-Gal-beta-4-Glc; and/or wherein the LNT-2 comprises GlcNAc-beta3-Gal-beta-4-Glc-beta.

9. The array of claim 1, wherein the library of glycans further comprises one or more of the following glycans:
Glycan
AGP α-acid glycoprotein
AGPAα-acid glycoprotein glycoformA
AGPBα-acid glycoprotein glycoformB
Ceruloplasmine
Fibrinogen
Transferrin
(Ab4[Fa3]GNb)2#sp1 LeX
(Ab4[Fa3]GNb)3#sp1 LeX
(Ab4GNb)3#sp1 Tri-LacNAc
[3OSO3]Ab#sp2 3SuGal
[3OSO3]Ab3ANa#sp2 3′SuGalβ3GalNAc
[3OSO3]Ab3GNb#sp2 3′SuGalβ3GalNAc
[3OSO3]Ab4[6OSO3]Gb#sp1 3′6DiSuLac
[3OSO3]Ab4[6OSO3]Gb#sp2 3′6DiSuLac
[3OSO3]Ab4Gb#sp2 3′SuLac
[3OSO3]Ab4GNb#sp2 3′SuLacNAc
[4OSO3]Ab4GNb#sp2 4′SuLacNAc
[6OPO3]Ma#sp2 6Pman
[6OSO3]Ab4[6OSO3]Gb#sp2 6′6DiSuLac
[6OSO3]Ab4Gb#sp1 6′SuLac
[6OSO3]Ab4Gb#sp2 6′SuLac
[6OSO3]GNb#sp2 6SuGlcNAc
[GNb3[GNb6]GNb4]ANa#sp2
[NNa3Ab]2GNb#sp2 (Sia)2GlcNAc
3OSO3Ab3[Fa4]GNb#sp2 3′SuLe a
3OSO3Ab4[Fa3]GNb#sp2 3′SuLe X
9NAcNNa#sp2 9NAc-Neu5Ac
9NAcNNa6Ab4GNb#sp2 9NAc-Neu5Ac2,6LacNAc
Aa#sp2 Galα
Aa2Ab#sp2 Galα2Gal
Aa3[Aa4]Ab4GNb#sp2 Galα3[Galα4]LacNAc
Aa3[Fa2]Ab#sp2 Galα3[Fuc]Galβ
Aa3Ab#sp2 Galα3Gal
Aa3Ab4[Fa3]GN#sp2 Galα3Le X
Aa3Ab4Gb#sp1 Galα3Lac
Aa3Ab4GN#sp2 Galα3LacNAc
Aa3Ab4GNb#sp2 Galα3LacNAc
Aa3ANa#sp2 Galα3GalNAc
Aa3ANb#sp2 Galα3GalNAc
Aa4[Fa2]Ab4GNb#sp2 Galα4[Fucα2]LacNAc
Aa4Ab4Gb#sp1 Galα4Lac
Aa4Ab4GNb#sp1 Galα4LacNAc
Aa4Ab4GNb#sp2 Galα4LacNAc
Aa4GNb#sp2 Galα4GlcNAc
Aa6Gb#sp2 Galα6Gal
Ab#sp2 Gal
Ab[NNa6]ANa#sp2 6Sialyl-T
Ab2Ab#sp2 Galβ2Gal
Ab3[Ab4GNb6]ANa#sp2 6LacNAc-Core2
Ab3[Fa4]GNb#sp1 Le a
Ab3[Fa4]GNb#sp2 Le a
Ab3[GNb6]ANa#sp2 Core-2
Ab3[NNa6]GNb4Ab4Gb#sp4 LSTc
Ab3[NNb6]ANa#sp2 β6Sialyl-T
Ab3Ab#sp2 Galβ3Gal
Ab3ANa#sp2 Galβ3GalNAcα
Ab3ANb#sp2 Galβ3GalNAcβ
Ab3ANb4[NNa3]Ab4Gb#sp1 GM1
Ab3ANb4Ab4Gb#sp2 a-sialo-GM1
Ab3GNb#sp1 LeC
Ab3GNb#sp2 LeC
Ab3GNb3Ab4Gb4b#sp4 LNT
Ab4[6OSO3]Gb#sp16SuLac
Ab4[6OSO3]Gb#sp2 6SuLac
Ab4[Fa3]GNb#sp1 LeX
Ab4[Fa3]GNb#sp2 LeX
Ab4ANa3[Fa2]Ab4GNb#sp2
Ab4Gb#sp1 Lac
Ab4Gb#sp2 Lac
Ab4GNb#sp1 LacNAc
Ab4GNb#sp2 LacNAc
Ab4GNb3[Ab4GNb6]Ana#sp2 (LacNAc)2-Core2
Ab4GNb3Ab4[Fa3]GNb3Ab4[Fa3]GNb#sp1 LacNAc-
LeX-LeX
Ab4GNb3Ab4Gb#sp1 LNnT
Ab4GNb3Ab4Gb#sp2 LNnT
Ab4GNb3Ab4GNb#sp1 LacNAc-LacNAc
Ab4GNb3ANa#sp2a 3LacANcα-Core-2
Ab4GNb3ANa#sp2b 3LacNAcβ-Core-2
Ab4GNb6ANa#sp2 6LacANcα-Core-2
Ana#sp2 Tn
Ana3[Fa2]Ab#sp2 A-tri
Ana3Ab#sp2 GalNAcα3Gal
Ana3Ab4GNb#sp2 GalNAcα3LacNAc
Ana3ANb#sp2 GalNAcα3GalNAc
Ana4[Fa2]Ab4GNb#sp2 GalNAcα4[Fucα2]LacNAc
ANb#sp2 GalNAcβ
ANb3[Fa2]Ab#sp2 GalNAcβ[Fucα2]Gal
ANb3Ana#sp2 GAlNAcβ3GalNAc
ANb4GNb#sp1 LacDiNAc
ANb4GNb#sp2 LacDiNAc
Fa#sp2 Fuc
Fa#sp3 Fuc
Fa2Ab#sp2 Fucα2Gal
Fa2Ab3[Fa4]GNb#sp2 Le b
Fa2Ab3Ana#sp2 H-type 3
Fa2Ab3ANb3Aa#sp3 H-type3β3Gal
Fa2Ab3ANb3Aa4Ab4G#sp3 Globo-H
Fa2Ab3ANb4[NNa3]Ab4Gb#sp1 Fucosyl-GM1
Fa2Ab3GNb#sp1 H-type 1
Fa2Ab3GNb#sp2 H type 1
Fa2Ab4[Fa3]GNb#sp1 Le Y
Fa2Ab4[Fa3]GNb#sp2 LeY
Fa2Ab4Gb#sp1 2′Flac
Fa2Ab4GNb#sp1 H-type 2
Fa2Ab4GNb#sp2 H-type 2
Fa2Ab4GNb3Ab4GNb#sp1 H-type-2-LacNAc
Fa2Ab4GNb3Ab4GNb3Ab4GNb#sp1 H-type2-LacNAc-
LacNAc
Fa2GNb#sp2 Fucα2GlcNAc
Fa3GNb#sp2 Fucα3GlcNAc
Fb3GNb#sp2 Fucβ3GlcNAc
Fa2Ab3ANb4[NNa3]Ab4Gb#sp3 Fucosyl-GM1
Ga#sp2 Galα
Ga4Gb#sp2 Galα4Gal
Gb#sp2 Galβ
Gb4Gb#sp2 Galβ4Gal
Gb6Gb#sp2 Galβ6Gal
GNb#sp1 GlcNAc
GNb#sp2 GlcNAc
GNb2Ab3ANa#sp2 GlcNAcβ2-Core-1
GNb3[GNb6]ANa#sp2 GlcNAcβ3[GlcNAcβ6GalNAc
GNb3Ab#sp2 GlcNAcβ3Gal
GNb3Ab3ANa#sp2 GlcNAcβ3-Core1
GNb3Ab4Gb#sp1 LNT-2
GNb3Ab4GNb#sp1 GlcNAcβ3LacNAc
GNb4[GNb6]ANa#sp2 GlcNAcβ4[GlcNAcβ6]GalNAc
GNb4GNb4GNb4b#sp2 Chitotriose
GNb4MDPLys
GNb6ANs#sp2 GlcANcβ6GalNAc
G-ol-amine glucitolamine
GUa#sp2 Glucurinic acidα
GUb#sp2 Glucuronic acidβ
Ka3Ab3GNb#sp1 KDNα2,3-type1
Ka3Ab4GNb#sp1 KDBα2,3-LacNAc
Ma#sp2 Mannose α
Ma2Ma2Ma3Ma#sp3
Ma2Ma3[Ma2Ma6]Ma#sp3
Ma2Ma3Ma#sp3
Ma3[Ma2Ma2Ma6]Ma#sp3
Ma3[Ma6]Ma#sp3 Man-3
Man-5#aa Man5-aminoacid
Man5-9 pool Man5-9-aminoacid
Man-6#aa Man6-aminoacid
Man-7#aa Man7-aminoacid
Man-8#aa Man8-aminoacid
Man-9#aa Man9-aminoacid
Na8Na#sp2 Neu5Acα2,8Neu5Ac
Na8Na8Na#sp2 Neu5Acα2,8Neu5Acα2,5Neu5Ac
NJa#sp2 Neu5Gc
NJa3Ab3[Fa4]GNb#sp1 Neu5GcLe a
NJa3Ab3GbN#sp1 Neu5Gc-type1
NJa3Ab4[Fa3]GNb#sp1 Neu5Gc-LeX
NJa3Ab4Gb#sp1 Neu5Gcα3Lactose
NJa3Ab4GNb#sp1 Neu5Gcα3LacNAc
NJa6Ab4GNb#sp1 Neu5Gcα6LacNAc
NJa6ANa#sp2 Neu5Gc6GalNAc (STn)
NNa#sp2 Neu5Ac
NNa3[6OSO3]Ab4GNb#sp2 3′Sia[6′Su]LacNAc
NNa3[ANb4]Ab4Gb#sp1 GM2
NNa3[ANb4]Ab4GNb#sp1 GM2(NAc)/CT/Sda
NNa3[ANb4]Ab4GNb2#sp1 sp1GM2(NAc)/CT/Sda
NNa3{Ab4[Fa3]GN}3b#sp1 Sia3-TriLeX
NNa3Ab#sp2 Neu5Acα2,3Gal
NNa3Ab3[6OSO3]Ana#sp2 Neu5Acα3[6Su]-T
NNa3Ab3[Fa4]GNb#sp2 SLe a
NNa3Ab3[NNa6]Ana#sp2 Di-Sia-T
NNa3Ab3ANa#sp2 3-Sia-T
NNa3Ab3GNb#sp1 Neu5Acα3Type-1
NNa3Ab3GNb#sp2 Neu5Acα3Type-1
NNa3Ab4[6OSO3]GNb#sp23′Sia[6Su]LacNAc
NNa3Ab4[Fa3][6OSO3]GNb#sp2 6Su-SleX
NNa3Ab4[Fa3]GNb#sp1 SleX
NNa3Ab4[Fa3]GNb#sp2 SleX
NNa3Ab4[Fa3]GNb3Ab#sp2 SleX penta
NNa3Ab4[Fa3]GNb3Ab4GNb#sp1 SleXLacNAc
NNa3Ab4Gb#sp1 3′Sialyllactose
NNa3Ab4Gb#sp2 3′Sialyllactose
NNa3Ab4GNb#sp1 3′SialyllacNAc
NNa3Ab4GNb#sp2 3′SialyllacNAc
NNa3Ab4GNb3Ab4GNb#sp1 3′SialylDiLacNAc
NNa3Ab4GNb3Ab4GNb3Ab4GNb#sp1 3′Sialyl-tri-
LacNAc
NNa3ANa#sp2 Siaα3GalNAc
NNa6Ab#sp2 Siaα6Gal
NNa6Ab4[6OSO3]]GNb#sp2 6′Sial[6Su]LacNAc
NNa6Ab4Gb#sp1 6′Sia-lactose
NNa6Ab4Gb#sp2 6′Sia-lactose
NNa6Ab4GNb#sp1 6′Sia-LacNAc
NNa6Ab4GNb#sp2 6′Sia-LacNAc
NNa6Ab4GNb3Ab4[Fa3]GNb3Ab4[Fa3]GNb#sp1 6Sia-
LacNAc-LeX-LeX
NNa6Ab4GNb3Ab4GNb#sp1 6SiaLacNAc-LacNAc
NNa6ANa#sp2 6SiaβGalNAc
NNa8NNa3[ANb4]Ab4Gb#sp1 GD2
NNa8NNa3Ab4Gb#sp1 GD3
NNa8NNa8NNa3[ANb4]Ab4Gb#sp1 GT2
NNa8NNa8NNa3Ab4Gb#sp1 GT3
NNAa3[NNa6]Ana#sp2 (Sia)2-Tn
NNb#sp2 Siaβ
NNb6Ab4GNb#sp2 6′SiaβLacNAc
NNb6ANa#sp2 βSTn
OS-11#sp2 6′sialLacNAc-biantenary glycan
Ra#sp2 Rhamnose
Abbreviations employed:
Sp1 = OCH2CH2NH2;
Sp2 = Sp3 = OCH2CH2CH2NH2
A = Gal; AN = GalNAc; G = Glc; GN = GlcNAc;
F = Fucose; NN; Neu5Ac (sialic acid);
NJ = Neu5Gc (N-glycolylsialic acid); a = α; b = β;
Su = sulfo; T = Galβ3GalNAc (T-antigen);
Tn = GalNAc (Tn-antigen).

10. The array of claim 1 comprising about 10 to about 200 glycans.

11. A breast cancer epitope selected from the group consisting of ceruloplasmin, Neu5Gc(2-6)GalNAc, GM1, Sulfo-T, Globo-H, LNT-2, Tri-LacNAc, LacNAc-LeX-LeX, LacNAc-LacNAc, H-type-2-LacNAc, H-type2-LacNAc-LacNAc, GlcNAcβ3LacNAc, SLeXLacNAc, 3′SialylDiLacNAc, 3′Sialyl-tri-LacNAc, 6Sia-LacNAc-LeX-LeX and 6SiaLacNAc-LacNAc glycans.

12. A composition comprising a carrier and an effective amount of at least one glycan molecule that binds antibodies associated with neoplasia of patients with benign, pre-malignant or malignant breast tumors.

13. The composition of claim 12, wherein the glycan molecule is selected from the group consisting of ceruloplasmin, Neu5Gc(2-6)GalNAc, GM1, Sulfo-T, Globo-H, sialylated Tn (Neu5Ac-alpha6-GalNAc-alpha), LNT-2, Tri-LacNAc, LacNAc-LeX-LeX, LacNAc-LacNAc, H-type-2-LacNAc, H-type2-LacNAc-LacNAc, GlcNAcβ3LacNAc, SLeXLacNAc, 3′SialylDiLacNAc, 3′Sialyl-tri-LacNAc, 6Sia-LacNAc-LeX-LeX and 6SiaLacNAc-LacNAc glycans.

14. The composition of claim 13, wherein the GM1 is Gal-beta3-GalNAc-beta-4-[Neu5Ac-alpha3]-Gal-beta-4-Glc-beta; wherein the Sulfo-T comprises a T-antigen with sulfate residues; wherein the Sulfo-T comprises Galβ3GalNAc, wherein sulfate can be present on one or more of the Sulfo-T galactose residues; wherein the Globo-H comprises Fucose-alpha2-Gal-beta3-GalNAc-beta3-Gal-alpha-4-Gal-beta-4-Glc; and/or wherein the LNT-2 comprises GlcNAc-beta3-Gal-beta-4-Glc-beta.

15. The composition of claim 13, which has at least two glycan molecules.

16. The composition of claim 13, which is formulated for immunization of a mammal.

17. The composition of claim 13, which is formulated for local administration to the breast.

18. The composition of claim 13, which is formulated as a food supplement.

19. A method of detecting breast cancer comprising contacting the array of claim 1 with a test sample obtained from a patient and observing whether antibodies in the test sample bind to at least one glycan molecule in the array that has previously determined to bind antibodies associated with neoplasia in patients with benign, pre-malignant or malignant breast tumors.

20. The method of claim 19, wherein the test sample is a blood sample, a serum sample, a plasma sample, a urine sample, a breast milk sample, an ascites fluid sample or a tissue sample.

21. The method of claim 19, wherein the glycan molecule is selected from the group consisting of ceruloplasmin, Neu5Gc(2-6)GalNAc, GM1, Sulfo-T, Globo-H, sialylated Tn (Neu5Ac-alpha6-GalNAc-alpha), LNT-2, Tri-LacNAc, LacNAc-LeX-LeX, LacNAc-LacNAc, H-type-2-LacNAc, H-type2-LacNAc-LacNAc, GlcNAcβ3LacNAc, SLeXLacNAc, 3′SialylDiLacNAc, 3′Sialyl-tri-LacNAc, 6Sia-LacNAc-LeX-LeX and 6SiaLacNAc-LacNAc glycans.

22. The method of claim 21, wherein the GM1 is Gal-beta3-GalNAc-beta-4-[Neu5Ac-alpha3]-Gal-beta-4-Glc-beta; wherein the Sulfo-T comprises a T-antigen with sulfate residues; wherein the Sulfo-T comprises Galβ3GalNAc; wherein the Globo-H comprises Fucose-alpha2-Gal-beta3-GalNAc-beta3-Gal-alpha-4-Gal-beta-4-Glc; and/or wherein the LNT-2 comprises GlcNAc-beta3-Gal-beta-4-Glc-beta.

23. The method of claim 19, which further comprises observing whether antibodies in a control sample bind to at least one glycan molecule that binds antibodies associated with neoplasia in patients with benign, pre-malignant or malignant breast tumors; wherein the control sample is from a patient that does not have breast cancer.

24. A method of detecting antibodies that bind breast cancer-related glycan epitopes comprising contacting a serum sample with the array of glycans of claim 1 and observing whether one or more glycans are bound by antibodies.

25. A method of treating or preventing breast cancer in a mammal that comprises administering to the mammal the composition of claim 12.

26. The method of claim 25, wherein the at least one glycan molecule is selected from the group consisting of ceruloplasmin, Neu5Gc(2-6)GalNAc, GM1, Sulfo-T, Globo-H, sialylated Tn (Neu5Ac-alpha6-GalNAc-alpha), LNT-2, Tri-LacNAc, LacNAc-LeX-LeX, LacNAc-LacNAc, H-type-2-LacNAc, H-type2-LacNAc-LacNAc, GlcNAcβ3LacNAc, SLeXLacNAc, 3′SialylDiLacNAc, 3′Sialyl-tri-LacNAc, 6Sia-LacNAc-LeX-LeX and 6SiaLacNAc-LacNAc glycans.

27. The method of claim 26, wherein the GM1 is Gal-beta3-GalNAc-beta-4-[Neu5Ac-alpha3]-Gal-beta-4-Glc-beta; wherein the Sulfo-T comprises a T-antigen with sulfate residues; wherein the Sulfo-T comprises Galβ3GalNAc; wherein the Globo-H comprises Fucose-alpha2-Gal-beta3-GalNAc-beta3-Gal-alpha-4-Gal-beta-4-Glc; and/or wherein the LNT-2 comprises GlcNAc-beta3-Gal-beta-4-Glc-beta.

28. The method of claim 25, wherein the composition has at least two glycan molecules.

29. The method of claim 25, wherein the composition is formulated for immunization of a mammal.

30. The method of claim 25, wherein the composition is locally administered to the mammal's breast.

31. The method of claim 25, wherein the composition is administered as a food supplement.

32. An isolated antibody that can bind the breast cancer epitope of claim 11.

33. The isolated antibody of claim 32, wherein the antibody can bind a glycan molecule selected from the group consisting of Tri-LacNAc, LacNAc-LeX-LeX, LacNAc-LacNAc, H-type-2-LacNAc, H-type2-LacNAc-LacNAc, GlcNAcβ3LacNAc, SLeXLacNAc, 3′SialylDiLacNAc, 3′Sialyl-tri-LacNAc, 6Sia-LacNAc-LeX-LeX and 6SiaLacNAc-LacNAc.

34. A method of treating or preventing breast cancer in a mammal that comprises administering to the mammal a composition comprising an effective amount of the antibody of claim 32.
